(19) United States
(12) Patent Application Publication
(10) Pub. No.: US 2019/0263867 A1
(43) Pub. Date: Aug. 29, 2019

(54) TAU PEPTIDES, METHODS OF MAKING, AND METHODS OF USING

(71) Applicant: REGENTS OF THE UNIVERSITY OF MINNESOTA, Minneapolis, MN (US)

(72) Inventor: Dezhi Liao, Minneapolis, MN (US)

(73) Assignee: REGENTS OF THE UNIVERSITY OF MINNESOTA, MINNEAPOLIS, MN (US)

(21) Appl. No.: 16/288,290

(22) Filed: Feb. 28, 2019

Related U.S. Application Data
(60) Provisional application No. 62/636,523, filed on Feb. 28, 2018.

(51) Int. Cl. C07K 7/08; C12N 15/62; C07K 14/16; A61P 25/28; A61P 25/16

(52) U.S. Cl. CPC C07K 7/08; C12N 15/62; A61K 38/00; A61P 25/28; A61P 25/16; C07K 14/163

(57) ABSTRACT

This disclosure describes a peptide including a tau peptide, methods of making the peptide, and methods of using the peptide. In some embodiments, the peptide prevents the mislocalization of tau that leads to tau-mediated synaptic deficits. In some cases, the peptide includes a protein transduction domain. In some embodiments, the peptide may be administered to a subject is at risk of or exhibiting symptoms of Alzheimer's Disease, Parkinson's disease, chronic traumatic encephalopathy, and/or another tauopathy.

### CONTINUING APPLICATION DATA

This application claims the benefit of U.S. Provisional Application Ser. No. 62/636,523, filed Feb. 28, 2018, which is incorporated by reference herein in its entirety.

### GOVERNMENT FUNDING

This invention was made with government support under grants NS084007 and NS096437 awarded by National Institutes of Health. The government has certain rights in the invention.

### SEQUENCE LISTING

This application contains a Sequence Listing electronically submitted to the United States Patent and Trademark Office via EFS-Web as an ASCII text file entitled “0110-000572US01_ST25.txt” having a size of 4 kilobytes and created on Feb. 27, 2019. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### BACKGROUND

Post-translational modifications of the cytoskeletal protein tau are implicated in synaptic dysfunction in Alzheimer's disease and other tauopathies. Long-lasting synaptic plasticity underpinning learning and memory involves the insertion of α-amino-3-hydroxy-5-methyl-4-isoxazolepropionic acid (AMPA) receptors into the postsynaptic membrane of dendritic spines and the removal of the receptors from spines. Under disease conditions, dendritic spines contain fewer AMPA receptors and elevated levels of modified, mislocalized tau. Proline-directed phosphorylation of serine (S) and threonine (T) residues in tau leads to postsynaptic dysfunction, but, at the time of the invention, details about cellular mechanisms remained unclear.

### SUMMARY OF THE INVENTION

This disclosure describes identification of the residues and modifications of tau involved in the mislocalization of tau and the reduction of AMPA receptors in dendritic spines, and therapies designed to interfere with those modifications and the subsequent mislocalization of tau and/or the reduction of AMPA receptors. In some aspects, such therapies may be useful in subjects at risk of or exhibiting symptoms of Alzheimer's Disease, Parkinson's disease, chronic traumatic encephalopathy, and/or another tauopathy.

In one aspect, this disclosure provides peptides, compositions including those peptides, and methods of using those peptides and compositions. In some embodiments, the peptide preferably prevents the mislocalization of tau that leads to tau-mediated synaptic deficits.

In some embodiments, the peptide includes a tau peptide. In some embodiments, the peptide includes a protein transduction domain and/or a modification to increase the peptide's ability to cross the blood-brain barrier.

In some embodiments, the tau peptide includes a sequence of amino acids having at least 80% homology to SPVVSGDTS (SEQ ID NO:4). In some embodiments, the tau peptide includes a sequence of amino acids that includes at least one of SPVVSGDTS (SEQ ID NO:4) and APVVSGDTA (SEQ ID NO:5). In some embodiments, the tau peptide includes a sequence of amino acids that includes at least one of KSPVVSGDTSP (SEQ ID NO:6) and KAPVVSGDTAP (SEQ ID NO:7).

In some embodiments, the tau peptide includes at least 9 amino acids, at least 10 amino acids, at least 11 amino acids, at least 12 amino acids, at least 13 amino acids, at least 14 amino acids, at least 15 amino acids, at least 18 amino acids, at least 20 amino acids, at least 22 amino acids, at least 25 amino acids, at least 26 amino acids, at least 27 amino acids, or at least 28 amino acids.

In some embodiments, the tau peptide includes DHGAEIVYKSPVVSGDTSPRHLSNVSST (SEQ ID NO:8). In some embodiments, the tau peptide includes DHGAEIVYKAPVVSGDTAPRHLSNVSST (SEQ ID NO: 9).

In some embodiments, the tau peptide includes a mutation that blocks the phosphorylation of at least one of S396 and S404 in tau. In some embodiments, at least one of S396 and S404 is replaced with an alanine.

In some embodiments, the protein transduction domain includes an HIV Trans-Activator of Transcription (TAT) domain. In some embodiments, the protein transduction domain includes GRKKRRQRRRPQ (SEQ ID NO: 10). In some embodiments, the protein transduction domain is conjugated to the N terminus of the tau peptide.

In some embodiments, the peptide reduces the localization of tau to the dendritic spines of a mechanically injured neuron by at least 10 percent.

In another aspect, this disclosure describes a method of making a peptide described herein.

In a further aspect, this disclosure describes a composition that includes a peptide described herein. In an additional aspect, this disclosure describes a virus encoding a peptide described herein.

In yet another aspect, this disclosure describes a method that includes administering a peptide, composition, or virus described herein. In some embodiments, the peptide may be administered via intraventricular injection or via intrathecal injection. In some embodiments, the virus may be introduced into a subarachnoid space.

In some embodiments, the method further includes administering a kinase inhibitor to the subject.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure.

Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A-FIG. 1E show blocking phosphorylation of C-residues reduces the mislocalization of P301L mutant tau to dendritic spines. FIG. 1A. An initial map for A-, B- and C-domains of tau (colored by orange, blue and pink, respectively; FIG. 6 shows the rationale for this grouping). T111, T153, T175, T181 and S199 constitute the A-residues in the A-domain; S202, T205, T212, T217 and T231 constitute the B-residues in the B-domain; and S235, S396, 5404 and S422 constitute the C-residues in the C-domain. FIG. 1B. Representative images of eGFP-tau constructs (green) and DsRed (red) expressed in rat primary hippocampal neuronal cultures. Tau expressing the P301L mutation mislocalized to a majority of spines, except when C-residues were mutated to alanine to block phosphorylation. “Wild type” or “−P301L” refers to tau without a P301L mutation and “native” refers to tau lacking mutations of the A-, B- or C-residues. “−Ala” refers to mutation to alanine. FIG. 1C. Quantification of percentage of spines containing tau. FIG. 1D. Spines containing P301L tau normalized to alanine variants with no P301L mutation to estimate the amount of tau missorting. FIG. 1E. Quantification of total spine density. For FIG. 1C. and FIG. 1E, data were analyzed by two-way ANOVA shielded Bonferroni post hoc analysis. In FIG. 1C, F(3, 112)=49.24; WT, A, B: *** P<0.0001; C: P>0.9999. In FIG. 1E, F (3, 112)=0.277. In FIG. 1D, data were analyzed by one-way ANOVA shielded Bonferroni post-hoc analysis; F(3,56)=72.03; *** P=<0.0001. For all, error bars represent mean±SD; n=15 neurons.

FIG. 2A-FIG. 2C show pseudophosphorylation of C-residues enhanced the mislocalization of wild-type tau to dendritic spines. FIG. 2A. Representative photomicrographs of eGFP-tau constructs (green) and DsRed (red) expressed in rat primary hippocampal neuronal cultures. “−Glu” indicates glutamate substitutions of S/T residues to mimic phosphorylation in the respective domains. The mislocalization of tau with pseudophosphorylated C-residues (4^{th} row) was comparable to that of P301L mutant tau (1^{st} row). The addition of B-Glu mutations to tau with C-Glu mutations did not further increase the mislocalization of tau (5^{th} row). FIG. 2B. Quantification of percentage of spines containing tau. FIG. 2C. Quantification of total spine density. Data were analyzed by two-way ANOVA shielded Bonferroni post hoc analysis. In FIG. 2B, F(3, 98)=40.45; *** P<0.0001. In FIG. 2C, F(3, 98)=0.699. n-values are represented parenthetically (number of neurons). Error bars represent mean±SD.

FIG. 3A-FIG. 3E show blocking phosphorylation of B- or C-residues prevents P301L mutant tau-induced glutamatergic postsynaptic deficits. FIG. 3A. Representative traces of rat hippocampal neurons transfected with eGFP-tau constructs. Neurons were bathed in artificial cerebral spinal fluid (ACSF) containing tetrodotoxin (TTX) (1 μM), picrotoxin (100 μM), and D, L-amino-5-phosphonovaleric acid (APV) (100 μM) to isolate AMPA receptor mini-excitatory postsynaptic currents (mEPSCs). P301L mutant tau-containing constructs led to a reduction in mEPSC amplitude, except when either the B-residues or the C-residues were mutated to alanine to prevent phosphorylation. FIG. 3B. Quantification of mEPSC amplitudes. (c) Quantification of mEPSC frequencies. FIG. 3D-FIG. 3G. Relative cumulative frequency of amplitudes of all mEPSC events in multiple groups. In b and c, data were analyzed by two-way ANOVA shielded Bonferroni post hoc analysis. In b, F(3, 79)=5.082; Native: *** P=0.0005, A: * P=0.0179. In c, F(3, 79)=0.4394. In d-g, data were analyzed by the Kolmogorov-Smirnov goodness-of-fit test; *** P<0.0001. Error bars represent mean±SD. n-values are represented in parentheses (number of neurons).

FIG. 4A-FIG. 4G show pseudophosphorylation of B- and C-residues combined induces glutamatergic postsynaptic deficits. FIG. 4A Representative traces of rat hippocampal neurons transfected with eGFP-tau constructs. Neurons were bathed in ACSF as before to isolate AMPA receptor mEPSCs. All P301L mutant tau-containing constructs show reduced mEPSC amplitudes. Pseudophosphorylation in no single domain induces deficits; however, pseudophosphorylation of B- and C-residues in combination reduced mEPSC amplitudes. FIG. 4B. Quantification of mEPSC amplitudes. (c) Quantification of mEPSC frequencies. FIG. 4D-FIG. 4G. Relative cumulative frequency of amplitudes of all mEPSC events in multiple groups. In FIG. 4B and FIG. 4C, data were analyzed by two-way ANOVA shielded Bonferroni post hoc analysis. In FIG. 4B, F(3, 76)=3.406; Native: *** P<0.0001, B: *** P=0.0004, C: ** P=0.0015. In FIG. 4C, F(3, 76)=0.5672.

In FIG. 4D-FIG. 4G, data were analyzed by the Kolmogorov-Smirnov goodness-of-fit test; *** P<0.0001. Error bars represent mean±SD. n-values are represented in parentheses (number of neurons).

FIG. 5A-FIG. 5F show blocking phosphorylation of S396 and S404 together prevented P301L mutant tau-induced mislocalization. FIG. 5A. Schematic representation of C-residues. Maroon color (S235 and S404) indicates residues phosphorylated by cyclin-dependent kinase 5 (cdk5); teal color (S396) indicates residues phosphorylated by glycogen synthase kinase 3 beta (gsk3β). Arrows point to a segment of tau that is highly phosphorylated under “normal” and disease conditions as reported by Mair et al. 2016 Anal Chem. 88, 3704-3714. FIG. 5B. Systematic mutagenesis of C-residues shows that blocking phosphorylation of S396 and S404 together prevented P301L mutant tau-induced mislocalization, but blocking either S396 or S404 alone did not prevent mislocalization. FIG. 5C. Percentage of spines containing tau in neurons that express eGFP-tau constructs (green) and DsRed (red); and had been treated with 500 nM roscovitine (cdk inhibitor) and/or CHIR99021 (gsk3β inhibitor). See FIG. 8 for representative images. FIG. 5D. Quantification of total spine density. FIG. 5E. Percent reduction in mislocalization from untreated P301L-tau by three drug treatments. FIG. 5F. Diagram illustrating a hypothetical model that integrates the interaction between phosphorylation by protein kinases (gsk3β and cdk5) and truncation by caspase-2. In FIG. 5B and FIG. 5E, data were analyzed by one-way ANOVA shielded Bonferroni post-hoc analysis. In FIG. 5B, F(6, 65)=74.32; *** P<0.0001. In FIG. 5E, F(2, 27)=28.08; *** P<0.0001. In FIG. 5C, data were analyzed by two-way ANOVA shielded Bonferroni post-hoc analysis; n=9-15, F(3, 77)=20.17; *** P<0.0001. For FIG. 5B and FIG. 5E, error bars represent mean±s.e.m. For FIG. 5C-FIG. 5D, error bars represent mean±SD.

FIG. 6 shows the evolution of hypotheses pertaining to the role of phosphorylation on postsynaptic dysfunction. Tau was partitioned based on Hypothesis 1 that phosphorylatable serine/phosphorylatable threonine (SP/TP) residues in the A-domain and/or B-domain activate calcineurin (see Yu et al. 2008 Biochim Biophys Acta. 1783, 2255-2261) leading to the internalization of AMPA receptors causing postsynaptic dysfunction (Step 1). The scientific rationale for this hypothesis was based on observations describing the interaction of a segment (aa198-244) of the proline rich region of tau (aa151-244) with the regulatory domains of the catalytic subunit of calcineurin. To test this hypothesis, SP/TP residues were mutated in the A-, B- or C-domains of wild type or P301L mutant tau to alanine to block phosphorylation (Step 2). The phosphorylation state of A-residues was neither necessary (FIG. 3) nor sufficient to cause synaptic deficits (FIG. 7). Next, the B- and C-domains were further characterized by producing tau variants with phosphomimetic substitutions in these domains (Step 3). Based on results shown in FIG. 1 to FIG. 4, the initial hypothesis was revised, and Hypothesis 2 was generated. The findings in FIG. 5 led to Hypothesis 3, a refinement of the second hypothesis.

FIG. 7A-FIG. 7C show analyses of exemplary mEPSCs recorded in neurons expressing A-Glu tau. FIG. 7A. Comparison of mEPSC amplitudes in neurons expressing native and A-Glu variants (neither contains the P301L mutation). FIG. 7B. Quantification of mEPSC frequencies. FIG. 7C. Relative cumulative frequency of amplitudes of all mEPSC events in both groups. NS: p>0.05. These results indicate that without more, phosphorylation of residues of the A domain is not sufficient to cause synaptic deficits. The results here and in FIG. 3 suggest that the phosphorylation of A domain plays a minimal role in tau-induced synaptic deficits over the time period observed (11-14 days after transfection). For FIG. 7A-FIG. 7B, data were analyzed with Student T-test. For FIG. 7C, data were analyzed by Kolmogorov-Smirnov goodness-of-fit test. Error bars are mean±s.e.m.

FIG. 8A-FIG. 8G show both gsk3β and cdk5 participate in P301L mutant tau-induced mislocalization to dendritic spines. FIG. 8A-FIG. 8C Representative images of neurons expressing eGFP-P301L mutant tau constructs (green) and DsRed (red) treated with 10-log concentrations of roscovitine (cdk inhibitor, FIG. 8B), CHIR99021 (gsk3β inhibitor, FIG. 8C), or both drugs (FIG. 8A). FIG. 8D-FIG. 8E. 10-log dose response curves showing percentage of spines containing tau in neurons that express eGFP-tau constructs and DsRed treated with roscovitine (FIG. 8D), and CHIR99021 (FIG. 8E). FIG. 8F-FIG. 8G. Quantification of percentage of spines containing tau in neurons treated with roscovitine (FIG. 8F) and CHIR99021 (FIG. 8G). Overt cell death was observed if drug concentrations exceeded 5 M (data not shown). Data were analyzed by two-way ANOVA shielded Bonferroni post-hoc analysis; *** P<0.001. n=8-14. Error bars are mean±SD.

FIG. 9A shows a cartoon illustration of the domains of tau and the phosphorylation sites (S396 and S404) involved in the missorting of tau to dendritic spines. Note that the S396 and S404 are the phosphorylation sites of gsk3β and cdk5, respectively. FIG. 9B shows the amino acid sequences of Peptide 1 (also referred to herein as wild-type (WT) peptide, SEQ ID NO: 1), Peptide 2 (also referred to herein as AP peptide, SEQ ID NO:2), and Peptide 3 (also referred to herein as EP peptide, SEQ ID NO:3). The peptides contain an HIV Trans-Activator of Transcription (TAT) domain (blue amino acids) and a sequence of amino acids that flank the S396 and S404 sites of tau (red amino acids). Note that the S396 and S404 sites are mutated to alanine in Peptide 2 (denoted by black “A”s) and to glutamic acid in Peptide 3 (denoted by black “E”s).

FIG. 10A-FIG. 10B show missorting of tau to dendritic spines caused by P301L mutation is blocked by Peptide 1 and Peptide 2 but not Peptide 3. FIG. 10A 21 days in vitro (DIV) neurons expressing DsRed (left panels) and GFP-tagged P301L-tau (middle panels) were treated with AP peptide (Peptide 2; 1 μM) for 3 days (top panels: untreated; bottom panels: treated). FIG. 10B The proportion of dendritic spines that contain tau was significantly decreased in neurons that had been treated with either WT peptide (Peptide 1) or AP peptide (Peptide 2), indicating that missorting of tau to dendritic spines is blocked by both peptides. AP peptide was observed to have a stronger effect. No decrease in the proportion of dendritic spines that contain tau was observed in neurons that had been treated with EP Peptide (Peptide 3).

FIG. 11A-FIG. 11B shows that mislocalization of tau to dendritic spines caused by Aβ oligomers is blocked by Peptide 2 (AP peptide). FIG. 11A Cultured 21 days in vitro (DIV) rat hippocampal neurons expressing DsRed (left panels) and GFP-tagged wild-type human tau (middle panels) were untreated (top panels), treated with Aβ oligomers alone (0.1 μM) (middle panels), or treated with Aβ oligomers (0.1 μM) with AP peptide (1 μM) (bottom panels) for 3 days. FIG. 11B The proportion of dendritic spines that contain tau was significantly increased by treatment with Aβ oligomers and this effect was blocked by Peptide 2 (AP peptide) (***, p<0.001, ANOVA).

FIG. 12A-FIG. 12C shows tau mislocalization can be caused by repeated small mechanical strains and is dependent upon tau phosphorylation caused by cdk5 and gsk3β. FIG. 12A. Representative live images of cultured rat hippocampal neurons that had been co-transfected with plasmids of DsRed (left lane; a red fluorescence protein to label dendritic spines) and green fluorescence protein (GFP)-tagged wild-type tau (GFP-WT-tau; middle lane) with overlay images on the right lane 2 days after the delivery of a mechanical protocol. The neurons were cultured on a plastic membrane and were stretched by a computer-controlled mechanical device, as described in Example 2 (10 stretches; 2% mechanical strain; 1 second interval between stretches) in 21 days in vitro (DIV). Arrows denote dendritic spines that contain tau and triangles denote dendritic spines devoid of tau. FIG. 12B. The proportion of dendritic spines containing tau was significantly increased after being stretched (black bar) and this damage was blocked by the AP peptide (gray bar; see characterization of this peptide in FIG. 10 and FIG. 11). FIG. 12C. The density of dendritic spines was not significantly changed by the application of mechanical force, indicating that tau mislocalization occurs at an early phase, suggesting a shared cellular mechanism between traumatic brain injury (TBI) and Alzheimer's disease (AD). ANOVA was used in FIG. 12B and FIG. 12C; *** indicates p<0.001.

FIG. 13A-FIG. 13J show A53T αS causes mutation-specific postsynaptic deficits in AMPAR signaling whereas overexpression of human αS variants, regardless of genotype, causes presynaptic suppression in acute hippocampal slices. FIG. 13A. A list of transgenic mice used in the Example 3. FIG. 13B, FIG. 13C. Immunoblots and quantification of human αS (HuSyn-1 antibody) and total (mouse and human) αS (BD Biosciences antibody 610787) in hippocampal lysates from 4-6 month-old MoPrP-Hu-αS transgenic and TgNg mice. Each lane represents an individual animal. αS levels are normalized to tubulin. 12-2 and H5 have comparable expression levels but have lower expression levels than G2-3 and O2. FIG. 13D. Input-output relationships of EPSCs (TgNg n=9, 12-2 n=10, H5 n=11, G2-3 n=9, O2 n=9); two-way ANOVA, F=0.29, P=1.0. FIG. 13E. Paired-pulse ratio induced by two consecutive stimuli delivered at different time intervals (TgNg n=15, 12-2 n=9, H5 n=11, G2-3 n=10, O2 n=11); two-way ANOVA, F=0.56, P=0.96. Representative traces are illustrated as insets, scale bars: 20 pA, 30 ms. FIG. 13F. Synaptic fatigue induced by 15 consecutive stimuli at 25 ms interpulse intervals (TgNg n=13, 12-2 n=8, H5 n=10, G2-3 n=13, O2 n=9); two-way ANOVA, F=0.57, P=0.99. Representative traces are illustrated as insets, scale bars: 40 pA, 70 ms. For D-F: two-way ANOVA with Fisher LSD post-hoc analysis. FIG. 13G. Representative AMPA and NMDA receptor-mediated synaptic response traces and AMPA to NMDA receptor current ratio (TgNg n=11, 12-2 n=7, H5 n=10, G2-3 n=8, O2 n=11). Scale bars: 20 pA, 100 ms. Kruskal-Wallis test with Dunn's method post-hoc analysis H=21.53, df=4; H5: Representative traces (FIG. 13H), mean amplitude (FIG. 13I), and mean frequency (FIG. 13J) of mEPSCs obtained in the presence of TTX (1 μM) (TgNg n=7, 12-2 n=10, H5 n=11, G2-3 n=8, O2 n=10). Scale bar: 5 pA, 2 s. One-way ANOVA with Fisher LSD post-hoc analysis, F=8.23, P<0.001 (amplitude); F=5.54, P=0.001 (frequency). For all, data are expressed as mean±s.e.m.; * P<0.05, ** P<0.01, and *** P<0.001 compared with TgNg, (#) P<0.05 and (##) P<0.01 compared with 12-2. TgNg control was taken from littermates of 12-2 mice. For all, n-values represent neurons, at least three 3-6 month old mice were used for every experimental condition.

FIG. 14A-FIG. 14G show A53T αS causes deficits in LTP and spatial learning and memory. FIG. 14A. Top panel, representative EPSC traces before (grey) and after (black) a high frequency stimulation (HFS) of the Schaffer collaterals recorded from TgNg, 12-2, H5 and G2-3 mice (scale bars: 10 pA, 15 ms). Bottom panel, EPSC amplitude vs. time obtained from the TgNg, 12-2, H5 and G2-3 mice (n=9, n=9, n=9 and n=8 respectively). Arrow head indicates HFS application. TgNg controls were taken from 12-2 littermates. FIG. 14B. EPSC amplitude pre- and 45 minute post-stimulation in the different mouse models. Within-group analysis: two-tailed paired t-test: t/df/P=−5.07/8/0.0010; −3.56/0.0074; −0.41/8/0.70; 0.39/6/0.71; for TgNg, 12-2, H5, and G2-3 respectively. Between group analysis: one-way ANOVA with a Fisher LSD post-hoc analysis F=3.54, P=0.027. At least three 3-6 month old mice were used for every experimental condition, n-values represent neurons. FIG. 14C. Diagram of the Barnes circular maze and representative occupancy plots from TgNg and G2-3 probe trials (color gradient bar plot, black: least occupied region, red: highest occupancy). FIG. 14D. Latency time to escape the maze during four consecutive training days. Two-way ANOVA, F(3, 51)=0.093. FIG. 14E. Mean distance from target, measured on each training day. Two-way ANOVA with Bonferroni post-hoc analysis, F(3, 51)=1.056; * P=0.030, ** P=0.0015. FIG. 14F. Mean time 11-12 month old TgNg and G2-3 animals spent in each quadrant of the maze during the probe trial. Two-way ANOVA with Bonferroni post-hoc analysis, F(3, 51)=5.34; *** P=0.0002. FIG. 14G. The average distance between the animals and the target during the probe trial. G2-3 mice were significantly more distant from the target than their TgNg littermates (TgNg, n=9; G2-3, n=10). Analyzed by Student t-test, t=4.50, df=17; *** P=0.0003. For all, data are expressed as mean±s.e.m.

FIG. 15A-FIG. 15F show A53T αS-induced postsynaptic deficits are independent of expression levels. FIG. 15A. Representative traces of events represented in C. (Scale bars: 5 pA, 2 s). FIG. 15B. Relative cumulative frequency of whole-cell mEPSC amplitudes from cultured transgenic mouse hippocampal neurons. Kolmogorov-Smirnov test; D=0.30, * P=0.048; D=0.34, ** P=0.0086; D=0.43, *** P=0.0002. Amplitude (FIG. 15C) and frequency (FIG. 15D) of mEPSCs. One-way ANOVA with Bonferroni post-hoc analysis, For FIG. 15C: F(4, 47)=4.48; H5:* P=0.014; G2-3: * P=0.026; ** P=0.0036. For FIG. 15D: F(4, 47)=3.54; H5: P=0.032; G2-3: P=0.031; O2: P=0.016. FIG. 15E. Representative images of eGFP-illuminated dendrites and spines from cultured Tg mouse hippocampal neurons. (Scale bar, 5 μm). FIG. 15F. Spine density of neurons represented in G. One-way ANOVA, F(4, 47)=2.42. Data are expressed mean±s.e.m; n-values are represented parenthetically.

FIG. 16A-FIG. 16F show A53T αS-induced postsynaptic deficits are cell autonomous.

FIG. 16A. Wide-field fluorescence photomicrographs from cultured rat hippocampal, DAPI-stained neurons expressing eGFP-tagged WT and A53T αS plasmids via calcium-phosphate transfection. The percentage of untransfected cells was tabulated. FIG. 16B. Photomicrographs of fixed neurons that had been transfected with eGFP, eGFP-WT αS or eGFP-A53T αS plasmids (left) and subsequently stained with a mouse anti-synaptophysin antibody (middle; with overlay on the right). Axons were visually traced and defined as thin, long neurites emerging from the soma with occasional perpendicular branch points. Arrows represent non-overlapping synaptophysin clusters, arrow heads point to synaptophysin-filled, eGFP-expressing synaptic boutons. The percentage of synaptophysin-clusters free of exogenous αS expression was calculated. FIG. 16C. Whole-cell AMPAR mEPSCs were recorded from cultured rat hippocampal neurons transfected with eGFP alone or eGFP-fused αS species (scale bar: 10 pA, 100 ms). FIG. 16D. Relative cumulative frequency of mEPSC amplitudes. Kolmogorov-Smirnov test, D=0.39; *** P<0.0001. E,F, Mean mEPSC amplitudes (FIG. 16D), and frequency (FIG. 16F). One-way ANOVA with Bonferroni post-hoc analysis, F(4,32)=3.65; * P=0.012. For all, data are expressed as mean±s.e.m.

FIG. 17A-FIG. 17K show stable and consistent expression of eGFP-fused human αS across constructs. FIG. 17A. Contour plots of flow cytometry gating parameters from the non-transfected group. FIG. 17B. Contour plots of two populations of cells in the non-transfected group: eGFP-negative, living cells (Q4); and eGFP-negative, dead cells (Q3). Contour plots of neurons transfected with eGFP-fused WT (FIG. 17C), A30P (FIG. 17D), E46K (FIG. 17E), and A53T (FIG. 17F) mutant human αS respectively. A small population of cells emerged that is both living and eGFP-positive (Q1). FIG. 17G. Histogram comparison of fluorescence in eGFP cell population. FIG. 17H. Mean eGFP fluorescence intensity from flow cytometer detection. Data were analyzed by one-way ANOVA, F(3, 3638)=2.38; n-values are eGFP-positive events, given parenthetically. There was no difference between the cellular distributions of αS variants. FIG. 17I. Deconvoluted example micrographs of an axon and a dendrite of a neuron expressing eGFP-WT αS. (Scale bar: 10 m). FIG. 17J-FIG. 17K. 15-image Z-series of dendrites and axons were analyzed to estimate cellular distribution of αS by using linear-analysis perpendicular to the shaft. Total area under the curve of dendritic (FIG. 17J) or axonal (FIG. 17K) fluorescence in each image-series was averaged and normalized to background fluorescence. One-way ANOVA, F(3,25)=0.45 (dendrite), F(3, 14)=0.90 (axon); n>4. For all, data are expressed as mean±s.e.m.

FIG. 18A-FIG. 18 C show A53T αS at two expression levels induces phosphorylation-dependent mislocalization of tau to dendritic spines. Neurons were cultured from TgNg, H5, and G2-3 hippocampi and transfected with DsRed to visualize cellular architecture, and eGFP-fused human tau to visualize subcellular location of tau. FIG. 18A. Representative photomicrographs of cultured TgNg, G2-3 and H5 hippocampal neurons expressing WT tau, AP tau (phosphorylation-blocking) or E14 tau (phosphomimetic). Scale bar: 10 m. FIG. 18B. Quantification of percentage of total dendritic spines containing tau. FIG. 18C. Spine density. For all, TgNg n=8, H5 n=6, G2-3 n=8; one-way ANOVA with Bonferroni post-hoc analysis, F(6, 47)=1.52; *** P<0.0001; data are expressed as mean±s.e.m.

FIG. 19A-FIG. 19D show A53T αS induces tau phosphorylation-dependent, cell-autonomous postsynaptic deficits. FIG. 19A. Representative traces of whole-cell mEPSCs recorded from cultured rat hippocampal neurons co-transfected with tau and αS variants. Scale bar: 5 pA, 100 ms. FIG. 19B. Relative cumulative frequency plot of mEPSC amplitude. FIG. 19C-FIG. 19D. Quantification of mean mEPSC amplitude (FIG. 19C) and mEPSC frequency (FIG. 19D) of co-transfected neurons. For all, n=12; two-way ANOVA with Bonferroni post-hoc analysis, F(1, 44)=4.86; ** P=0.0095. Data are expressed as mean±s.e.m.

FIG. 20A-FIG. 20 G show GSK3β activation is required for tau mislocalization and synaptic deficits in A53T αS-expressing neurons. FIG. 20A. Representative photomicrographs from cultured TgNg and G2-3 neurons that were either untreated or treated with GSK3β-specific inhibitor CHIR-99021 (CHIR). Scale bar: 5 μm. FIG. 20B-FIG. 20C. Quantification of spines containing tau (FIG. 20B), and spine density (FIG. 20C). Two-way ANOVA with Bonferroni post-hoc analysis, F(2, 42)=27.27. FIG. 20D. Representative mEPSC traces from untreated (top) and CHIR-treated neurons (bottom) expressing eGFP alone, eGFP-WT αS and eGFP-A53T αS (Scale bar: 10 pA, 100 ms). FIG. 20E. Relative cumulative frequency of mEPSC amplitudes from neurons represented in D. Kolmogorov-Smirnov comparison to eGFP; D=0.53 *** P<0.0001. FIG. 20F-FIG. 20G. Quantification of mEPSC amplitude (FIG. 20F), and frequency (FIG. 20G). Two-way ANOVA with Bonferroni post-hoc analysis. F(2, 66)=3.214; * P=0.041. For all n=12; data are expressed as mean±s.e.m.

FIG. 21A-FIG. 21B shows GluA1 surface expression in dendritic spines is decreased by A53T αS expression in a GSK3β dependent fashion. FIG. 21A. Photomicrographs of neurons from G2-3 mice and their TgNg littermates without (top two panels) and with treatment of CHIR-99021 (bottom two panels). As previously described (Liao et al. 1999 Nature Neuroscience 2(1): 37-43), live neurons were stained for N-GluA1 antibodies (green), fixed, permeablized, and stained for PSD-95 (red). Arrows indicate tightly clustered surface N-GluA1 colocalized with PSD-95, whereas weak, non-specific N-GluR1 immunoreactivity appeared along the dendritic shafts as diffuse staining rather than distinct clusters in G2-3 neurons (arrow heads). The diffuse staining is likely due to the presence of extrasynaptic AMPA receptors (Newpher and Ehlers 2008 Neuron 58: 472-97). Treatment with CHIR-99021 restored surface N-GluA1 synaptic localization in G2-3 mice. Scale bar: 10 am. FIG. 21B. GluA1 surface fluorescence in PSD-95 immunoreactive spines was normalized to dendritic fluorescence. Two-way ANOVA with Bonferroni post-hoc analysis, F(1, 28)=5.69; ** P=0.0029. For all n=8; data are expressed as mean±s.e.m.

FIG. 22A-FIG. 22D shows calcineurin activation is required for tau and A53T αS induced postsynaptic deficits. FIG. 22A. Representative mEPSC traces recorded from cultured rat hippocampal neurons transfected with WT αS, treated with DMSO vehicle; or with A53T αS, treated with DMSO vehicle or FK506 (Scale bar: 10 pA, 100 ms). FIG. 22B. Relative cumulative frequency of mEPSC amplitudes from neurons represented in A. Kolmogorov-Smirnov comparison to vehicle-treated neurons expressing eGFP-WT αS, D=0.43 *** P<0.0001. FIG. 22C-FIG. 22D. Quantification of mEPSC amplitude (FIG. 22C) and frequency (FIG. 22D). One-way ANOVA with Bonferroni post-hoc analysis, F(4, 41)=3.84; ** P=0.0025; for all n=12. All data are expressed mean±s.e.m.

FIG. 23 shows hypothetical pathways for αS-induced changes in neuronal transmission: In pathway #1, A53T αS induces mutation specific, GSK3β-dependent phosphorylation of tau, leading to tau missorting to dendritic spines. Here, tau leads to calcineurin (CaN)-mediated endocytosis of GluA1-containing AMPA receptors leading to post-synaptic deficits. However, tau-mediated inhibition of AMPA receptor insertion into the synaptic membrane cannot be ruled out. In pathway #2, hyperexpression of WT or mutant αS (A53T, A30P) leads to presynaptic release suppression through an unknown mechanism regardless of genotypes. The differential effects of αS on these two separate pathways may contribute to PD heterogeneity.

### DETAILED DESCRIPTION OF ILLUSTRATIVE EMBODIMENTS

This disclosure provides a peptide, compositions including the peptide, and methods of using the peptide and compositions. In some embodiments, the peptide prevents the mislocalization of tau that leads to tau-mediated synaptic deficits. In one embodiment, the peptide interferes with the phosphorylation of S396 and/or S404 in the C-terminal tail of tau. In some aspects, the peptides may be used as a therapy in subjects at risk of or exhibiting symptoms of Alzheimer's Disease, Parkinson's disease, chronic traumatic encephalopathy, and/or another tauopathy.

Post-translational modifications of the cytoskeletal protein tau are implicated in neurodegenerative diseases including Alzheimer's disease (AD) and other tauopathies including, for example, frontotemporal dementia with parkinsonism-17 (FTDP-17) and chronic traumatic encephalopathy (CTE). Tau is a multifunctional protein having an unstructured form that enables it to interact with many different proteins. At the time of the invention, however, little was known about how phosphorylation in different regions of tau related to postsynaptic dysfunction, and previous attempts to treat AD by blocking tau have had limited success.

Previous studies have shown tau mislocalization to dendritic spines plays a role in functional deficits (Hoover et al. 2010 Neuron 68(6):1067-1081; Ittner et al. 2010 Cell 142, 387-397), but the involvement of specific phosphorylation sites has not yet been defined. Example 1 describes a previously unreported pathway by which tau induces synaptic dysfunction in tauopathies by targeting specific phosphorylatable serine/phosphorylatable threonine (SP/TP) residues. As further described in Example 1, phosphorylation in two non-overlapping tau domains regulates a two-step process leading to postsynaptic dysfunction. First, the phosphorylation of S396 or S404 in the C-terminal tail of tau results in tau mislocalization to dendritic spines. Second, the phosphorylation of one or more residues in the proline-rich region of tau (the B domain) results in the decrease of AMPA receptors in the dendritic spines.

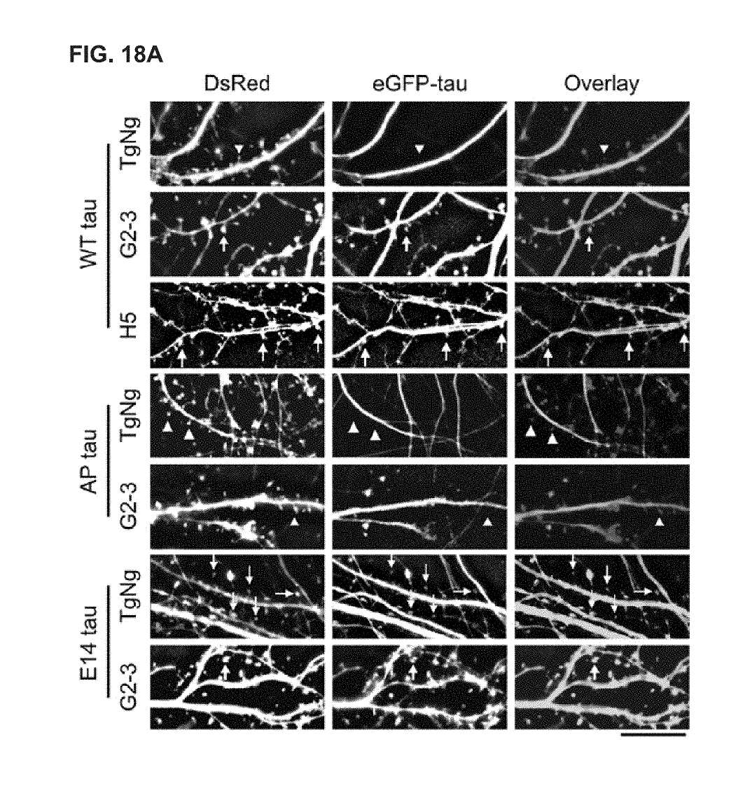

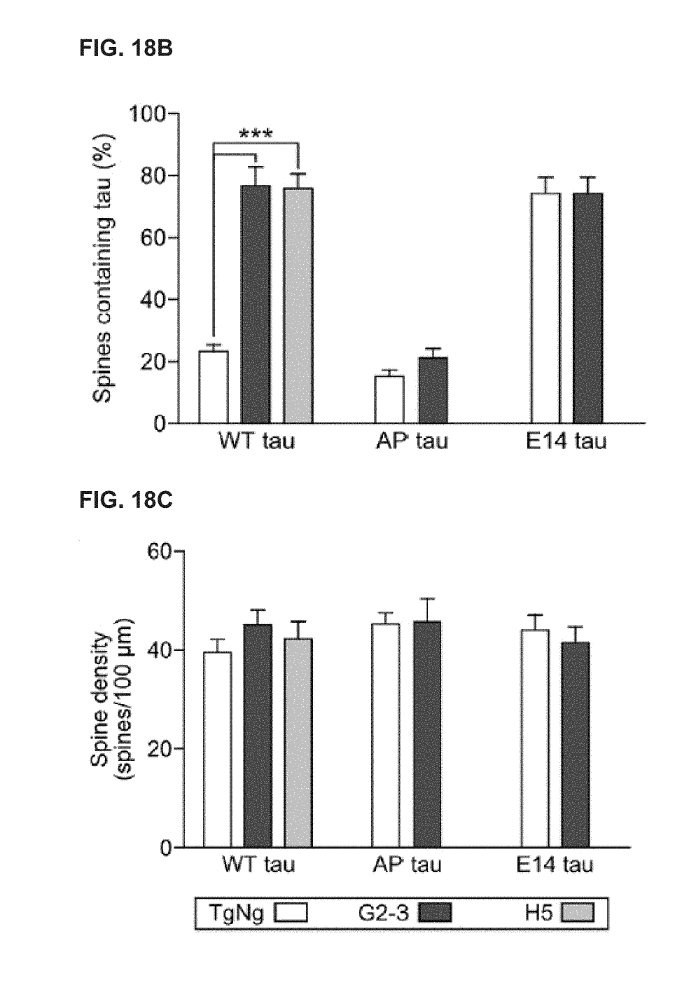

Example 3 describes a role for alpha-synuclein (αS) in tau missorting to dendritic spines and subsequent loss of postsynaptic AMPA receptors. In particular, A53T αS, a mutation of αS associated with familial Parkinson's disease, induces postsynaptic deficits that require GSK3β-dependent tau missorting to dendritic spines and calcineurin-dependent loss of postsynaptic surface AMPA receptors. As described in Example 3, when residues of tau were converted to unphosphorylatable residues, tau no longer mislocalized to dendritic spines even when A53T αS was expressed (se FIG. 18B), indicating that tau phosphorylation is necessary for A53T αS-induced mislocalization to dendritic spines.

The findings of Example 1 and Example 3 suggest that preventing either the mislocalization of tau or the reduction of AMPA receptors in dendritic spines including, for example, by targeting the specific post-translational modifications (e.g., phosphorylation) involved may provide promising therapies for tauopathies.

### Peptides

Thus, in one aspect, this disclosure provides peptides, compositions including those peptides, and methods of using those peptides and compositions to prevent the mislocalization of tau that leads to tau-mediated synaptic deficits.

In one aspect, this disclosure describes a peptide. In some embodiments, the peptide prevents the mislocalization of tau that leads to tau-mediated synaptic deficits. In some embodiments, the peptide reduces the localization of tau to dendritic spines of a mechanically injured neuron by at least 10 percent, at least 20 percent, at least 30 percent, at least 40 percent, at least 50 percent, at least 60 percent, at least 70 percent, at least 80 percent, or at least 90 percent.

In one embodiment, the peptide interferes with the phosphorylation of S396 and/or S404 in the C-terminal tail of tau.

In some embodiments, the peptide includes a tau peptide. A tau peptide includes amino acids contained in the tau protein. In some embodiments, the tau peptide includes a peptide having at least 70% homology, at least 75% homology, at least 80% homology, at least 85% homology, at least 90% homology, or at least 95% homology to the corresponding amino acids in the tau protein. In some embodiments, the tau peptide includes at least some of the amino acids of tau between positions S396 and S404. In some embodiments, the tau peptide includes a sequence of amino acids including each of the amino acids of tau between positions S396 and S404. For example, in some embodiments, the tau peptide includes PVVSGDT (SEQ ID NO:11). In some embodiments, the tau peptide includes a sequence having at least 70% homology to SPVVSGDTS (SEQ ID NO:4). In some embodiments, the tau peptide includes a peptide that includes amino acids of the tau protein except that the serine at least one of positions 369 and 404 of tau are replaced with an alanine. For example, in some embodiments, the tau peptide includes at least one of APVVSGDTS (SEQ ID NO: 12), SPVVSGDTA (SEQ ID NO: 13) and APVVSGDTA (SEQ ID NO:5).

In some embodiments, the tau protein is preferably human tau protein.

In some embodiments, the tau peptide includes at least 9 amino acids, at least 10 amino acids, at least 11 amino acids, at least 12 amino acids, at least 13 amino acids, at least 14 amino acids, at least 15 amino acids, at least 18 amino acids, at least 20 amino acids, at least 22 amino acids, at least 25 amino acids, at least 26 amino acids, at least 27 amino acids, or at least 28 amino acids.

In some embodiments, the tau peptide includes up to 10 amino acids, up to 12 amino acids, up to 13 amino acids, up to 14 amino acids, up to 15 amino acids, up to 18 amino acids, up to 20 amino acids, up to 22 amino acids, up to 25 amino acids, up to 26 amino acids, up to 27 amino acids, up to 28 amino acids, up to 30 amino acid, up to 31 amino acids, up to 35 amino acids, up to 40 amino acids, up to 45 amino acids, up to 50 amino acids, or up to 100 amino acids.

In some embodiments, the tau peptide includes a sequence including SPVVSGDTS (SEQ ID NO:4); in some embodiments, the tau peptide includes a sequence including APVVSGDTA (SEQ ID NO:5). In some embodiments, the tau peptide the peptide includes a sequence including KSPVVSGDTSP (SEQ ID NO:6); in some embodiments, the tau peptide the peptide includes a sequence including KAPVVSGDTAP (SEQ ID NO:7).

In some embodiments, the tau peptide includes a sequence including DHGAEIVYKSPVVSGDTSPRHLSNVSST (SEQ ID NO:8). In some embodiments, the tau peptide includes a sequence having 80% sequence identity, 85% sequence identity, 90% sequence identity, 95% sequence identity, 97% sequence identity, 98% sequence identity, or 99% sequence identity to the sequence including DHGAEIVYKSPVVSGDTSPRHLSNVSST (SEQ ID NO:8). In some embodiments, the tau peptide includes a sequence consisting of DHGAEIVYKSPVVSGDTSPRHLSNVSST (SEQ ID NO:8).

In some embodiments, the tau peptide includes a mutation that blocks the phosphorylation of at least one of S396 and S404. In some embodiments, at least one of S396 and S404 is replaced with an alanine. In some embodiments, the tau peptide includes a sequence comprising DHGAEIVYKAPVVSGDTAPRHLSNVSST (SEQ ID NO: 9). In some embodiments, the tau peptide includes a sequence consisting of DHGAEIVYKAPVVSGDTAPRHLSNVSST (SEQ ID NO: 9).

In some embodiments, the peptide includes a protein transduction domain, that is, a sequence to make the peptide membrane permeable. In some embodiments, the peptide includes an HIV Trans-Activator of Transcription (TAT) protein transduction domain. In some embodiments an HIV Trans-Activator of Transcription (TAT) domain sequence can include GRKKRRQRRRPQ (SEQ ID NO: 10). In some embodiments, a protein transduction domain can include a cationic peptide sequence including, for example, a sequence including predominantly arginine, ornithine and/or lysine residues. In some embodiments, a protein transduction domain can include a hydrophobic sequence including, for example, a leader sequence. In some embodiments, the protein transduction domain is conjugated to the N-terminus of the tau peptide. In some embodiments, the protein transduction domain is conjugated to the C-terminus of the tau peptide. In some embodiments, a linker sequence may be included between a protein transduction domain and the tau peptide.

In some embodiments, the chemical structure of the peptide may be modified to increase specificity and/or blood-brain barrier crossing ability.

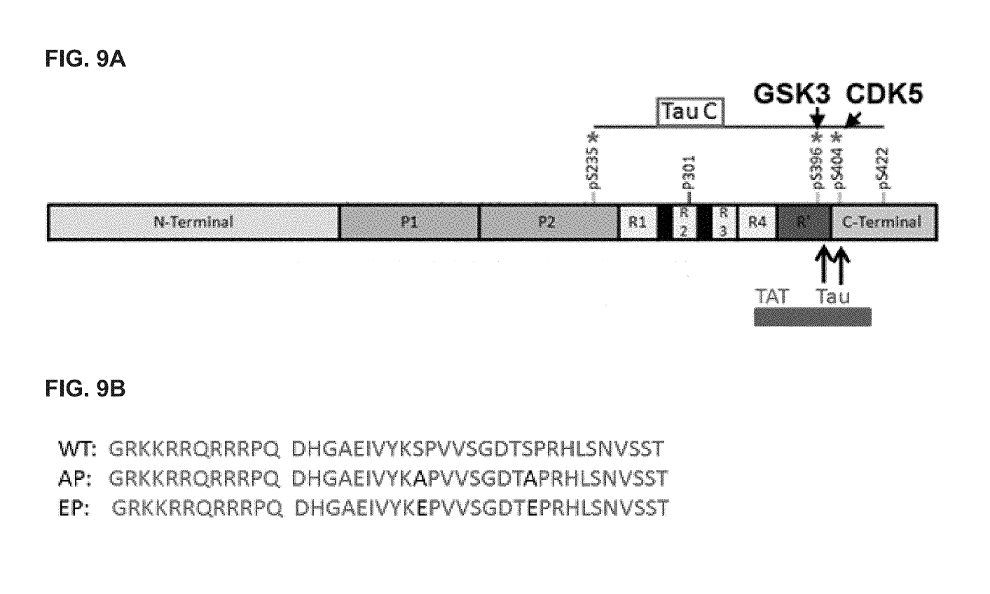

In some embodiments, including for example, when the peptide does not include a protein transduction domain, the peptide includes a modification to increase its ability to cross the blood-brain barrier. For example, the peptide may be conjugated to a blood-brain barrier shuttle. (Malakoutikhah et al. 2011 Angew Chem Int Ed Engl. 50(35):7998-8014.) In some embodiments, the structure of the peptide may be altered to increase its chemical stability. Modifications may include, for example, modification of peptide bonds, introduction of nonproteinogenic amino acids, and/or modification of the amino acid side chains and/or terminal residues. (See, for example, Peptide Modifications to Increase Metabolic Stability and Activity, Cudic (ed.), Humana Press (2013).) Exemplary embodiments of peptides that prevent the mislocalization of Tau are described in FIG. 9 and Example 2. For example, two peptides that block mislocalization of tau include:’
H2N-GRKKRRQRRRPQDHGAEIVYKSPVVSGDTSPRHLSNVS ST-OH (Peptide 1; wild-type form; also referred to herein as WT peptide, SEQ ID NO: 1) and
H2NGRKKRRQRRRPQDHGAEIVYKAPVVSGDTAPRHLSNVSST-OH (Peptide 2; blocking form, with the AP mutation; also referred to herein as AP peptide, SEQ ID NO:2).

In another aspect, this disclosure describes methods of making the peptides described herein. The peptide may be synthesized by any suitable method. For example, in some embodiments, the peptide may be chemically synthesized using a solid phase peptide synthesis (SPPS) technique by incorporating amino acids into a peptide of any desired sequence.

In some embodiments, the peptide may be biologically expressed by an appropriate vector (plasmid or virus).

### Pharmaceutical Compositions

In a further aspect, this disclosure describes compositions including a peptide described herein. The compositions may be suitable for oral, rectal, vaginal, topical, nasal, ophthalmic or parenteral (including subcutaneous, intramuscular, intraperitoneal, and intravenous) administration.

A composition may also include, for example, buffering agents to help to maintain the pH in an acceptable range or preservatives to retard microbial growth. A composition may also include, for example, carriers, excipients, stabilizers, chelators, salts, or antimicrobial agents. Acceptable carriers, excipients, stabilizers, chelators, salts, preservatives, buffering agents, or antimicrobial agents, include, but are not limited to, buffers such as phosphate, citrate, and other organic acids; antioxidants including ascorbic acid and methionine; preservatives, such as sodium azide, octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; polypeptides; proteins, such as serum albumin, gelatin, or non-specific immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN, PLURONICS, or polyethylene glycol (PEG).

The composition may be presented in unit dosage form and can be prepared by any of the methods well-known in the art of pharmacy. In some embodiments, a method includes the step of bringing the peptide into association with a pharmaceutical carrier. In general, a composition may be prepared by uniformly and intimately bringing the peptide into association with a liquid carrier, a finely divided solid carrier, or both, and then, if necessary, shaping the product into a desired formulation.

Methods of Administration and/or Treatment

A peptide described herein may be administered to a subject alone or in a pharmaceutical composition. In some embodiments, the peptide may be administered to a subject by introducing a vector (for example, a virus or plasmid) encoding the peptide into the subject. In some embodiments, the vector may be an adenovirus. The peptide or vector may be delivered using any suitable method. The subject may be an animal or a human. In some embodiments, the subject may be at risk of or may exhibit symptoms of Alzheimer's Disease, Parkinson's disease, chronic traumatic encephalopathy, and/or another tauopathy.

The peptide, a composition including the peptide, or a vector encoding the peptide can be administered to a vertebrate, more preferably a mammal, such as a human patient, in an amount effective to produce the desired effect. A peptide, a composition including the peptide, or a vector encoding the peptide can be administered in a variety of routes, including orally, parenterally, intraperitoneally, intravenously, intraarterially, transdermally, sublingually, intramuscularly, rectally, transbuccally, intranasally, liposomally, via inhalation, intraoccularly, via local delivery by catheter or stent, subcutaneously, intraadiposally, intraarticularly, intrathecally, or in a slow release dosage form.

A formulation can be administered as a single dose or in multiple doses. Useful dosages of a peptide, a composition including the peptide, or a vector encoding the peptide can be determined by comparing their in vitro activity and the in vivo activity in animal models. Methods for extrapolation of effective dosages in mice, and other animals, to humans are known in the art.

In some embodiments, the peptide may preferably be injected. For example, the peptide may be injected into the brain including, for example, into a ventricle. In some embodiments, the peptide may preferably be introduced via intrathecal injection.

In some embodiments, including, for example, when a vector encoding the peptide is administered, the peptide may preferably be administered by injection into a subarachnoid space.

In some embodiments, including, for example, to treat traumatic brain injuries, the peptides may be transfused into the cerebrospinal ventricular system.

In some embodiments, a peptide, a composition including the peptide, or a vector encoding the peptide may be administered to a subject in combination with a kinase inhibitor. For example, a kinase inhibitor may include at least one of a calcineurin inhibitor including, for example, cyclosporin, voclosporin, pimecrolimus and tacrolimus (FK506); a cdk5 inhibitor; and a gskβ inhibitor including, for example, tideglusib. The kinase inhibitor may be administered by any suitable method.

Dosage of a peptide, a composition including the peptide, or a vector encoding the peptide may be varied so as to obtain an amount of the active agent which is effective to achieve the desired therapeutic response for a particular subject, composition, and mode of administration, without being toxic to the subject. The selected dosage level will depend upon a variety of factors including, for example, the route of administration, the time of administration, the rate of excretion of the particular compound being employed, the duration of the treatment, other drugs, compounds and/or materials used in combination with the aurone, the age, sex, weight, condition, general health and prior medical history of the subject being treated, and like factors well known in the medical arts. A physician or veterinarian having ordinary skill in the art may determine and prescribe the effective amount of the pharmaceutical composition, peptide, or a vector encoding the peptide required.

A peptide, a pharmaceutical composition including the peptide, or a vector encoding the peptide may be used to treat or prevent a tauopathy. Exemplary tauopathies include but are not limited to Alzheimer's Disease, A53T α-synuclein-associated familial Parkinson's disease, traumatic brain injury (TBI), and other diseases that include tau missorting.

In some embodiments, this disclosure provides a therapeutic method of treating a subject suffering from a tauopathy by administering a peptide, a pharmaceutical composition including the peptide, or a vector encoding the peptide to the subject. Therapeutic treatment is initiated after diagnosis or the development of symptoms of tauopathy.

In some embodiments, this disclosure provides a method of treating a subject prophylactically, to prevent or delay the development of a tauopathy. Treatment that is prophylactic, for instance, can be initiated before a subject manifests symptoms of a tauopathy. An example of a subject that is at particular risk of developing a tauopathy is a person who has suffered traumatic brain injury (TBI) or a person having a A53T mutation in α-synuclein, which causes familial Parkinson's disease. Treatment may be performed before, during, or after the diagnosis or development of symptoms of a tauopathy. Treatment initiated after the development of symptoms may result in decreasing the severity of the symptoms, or completely removing the symptoms.

Administration of the peptide, a pharmaceutical composition including the peptide, or a vector encoding the peptide to the subject can occur before, during, and/or after other treatments. The present invention is illustrated by the following examples. It is to be understood that the particular examples, materials, amounts, and procedures are to be interpreted broadly in accordance with the scope and spirit of the invention as set forth herein.

### EXAMPLES

### Example 1—Phosphorylation in Two Discrete Tau Domains Regulates a Stepwise Process Leading to Postsynaptic Dysfunction

This Example describes the characterization of phosphorylation in two non-overlapping tau domains that can regulate a two-step process leading to postsynaptic dysfunction. First, tau mislocalizes to dendritic spines, and this process depends on the phosphorylation of S396 or S404 in the C-terminal tail of tau. Second, AMPA receptors in the spines are diminished, a reduction that involves both the mislocalization of tau and the phosphorylation of one or more of five SP/TP residues (S202, T205, T212, T217, and T231) in the proline-rich region of tau.

### Materials and Methods

### Materials

All common chemical reagents and cell culture supplies were purchased from Sigma-Aldrich (St. Louis, Mo.), Promega (Madison, Wis.), and Thermo-Fisher Scientific/Invitrogen/Life Technologies (Waltham, Mass.) unless otherwise indicated.

### Plasmids

All human tau and dsRed constructs were expressed in the pRK5 vector and driven by the cytomegalovirus promotor (Takara Bio USA (formerly known as Clontech Laboratories, Inc.), Mountain View, Calif.). All human tau was n-terminally fused to enhanced GFP (eGFP). The wild type, native human tau construct encoded human four-repeat tau lacking the transcriptional-variant n-terminal sequences (0N4R) and contained exons 1, 4, 5, 7, 9-13, 14 and intron 13. P301L mutant as well as alanine and glutamate tau variant constructs were created using step-wise site-directed mutagenesis (QuikChange Site-Directed Mutagenesis Kit, Agilent Technologies, Santa Clara, Calif.). PCR primers for mutagenesis were 15-22 nucleotides long, centered on mutated nucleotide(s) (Integrated DNA Technologies, Coralville, Iowa). All nucleotide mutations as well as plasmid construct integrity were confirmed with Senger Sequencing (University of Minnesota Genomics Center, Minneapolis, Minn.). Δtau314 constructs were generated and confirmed as discussed in Zhao, X. et al. 2016 Nat. Med. 22, 1268-1276. Tau sequence numbering was based on the longest functional human isoform: 441-tau (2N4R tau; NCBI reference sequence: NP_005901.2).

### Primary Hippocampal Neuron Cultures

Dissociated rat primary hippocampal neuron cultures described in this study were conducted in accordance with the American Association for the Accreditation of Laboratory Animal Care and Institutional Animal Care and Use Committee at the University of Minnesota (protocol #1211A23505). Briefly, a 25 mm diameter glass coverslip (0.08 mm thickness) was silicone-sealant-fastened to the bottom of a 35 mm culture dish with a bored hole and sterilized. Coverslips were coated with poly-D-lysine. Hippocampi were dissected from CO₂-anesthetized neonatal Sprague-Dawley timed-pregnancy rats (Envigo Corporation, Huntingdon, UK) at 0-24 hours of life. Hippocampi were enzymatically digested in Earle's Balance Salt Solution (EBSS) supplemented with 1% glucose and cysteine-activated papain. Digestion was blocked with dilute DNase, and cells were rinsed in fresh EBSS and plated in plating medium (minimal essential medium with Earle's salts, 10% fetal bovine serum, 2 mM glutamine, 10 mM sodium pyruvate, 10 mM HEPES, 0.6% glucose, 100 U/ml penicillin and 100 mg/mL streptomyocin) at 1.0×10⁶ cells/dish. After 18 hours, cell adherence was established. Cells were then grown in neurobasal medium (NbActiv1; BrainBits LLC, Springfield, Ill.) and incubated at 37° C. in a 5% CO₂ biological incubator.

### Low Efficiency Calcium-Phosphate Transfection

After 5-7 days in vitro (DIV), cells were transfected. DNA plasmid transfection was performed using standard calcium phosphate precipitation and incubation. Briefly, neurons were transfected with human tau constructs and dsRed (2:1 by plasmid DNA mass) for imaging experiments, and with human tau alone for electrophysiology. Precipitated DNA was applied to cells in a solution of glial conditioned medium (neurobasal medium previously conditioned for 14 days on a glial monolayer and reserved) containing 100 μM APV to prevent calcium-toxicity. After 3-4 hours transfection time, cells were rinsed in glial conditioned medium and grown in neurobasal medium as described above until mature (21-28 total DIV).

### Electrophysiolology

Miniature EPSCs were recorded from cultured dissociated rat hippocampal neurons at 21-25 DIV with a glass pipette (resistance ˜5 MΩ) at holding potentials of −65 mV on an Axopatch 200B amplifier (output gain=1; filtered at 1 kHz; made by Molecular Devices, San Jose, Calif.). Input and series resistances were assessed before and after recording mEPSCs (5-20 minutes) and found to have no significant difference before and after recording. Recording sweeps lasted 200 ms and were sampled for every 1 s (pCLAMP; Molecular Devices, San Jose, Calif.). Neurons were bathed in bubble-oxygenated artificial cerebral spinal fluid (ACSF) at 23° C. with 100 μM APV (NMDAR antagonist), 1 μM TTX (sodium channel blocker), and 100 μM picrotoxin (GABAa receptor antagonist). Passive oxygen perfusion was established with medical-grade 95% O₂-5% CO₂. ACSF contained (in mM) 119 NaCl, 2.5 KCl, 5.0 CaCl₂, 2.5 MgCl₂, 26.2 NaHCO₃, 1 NaH₂PO₄, and 11 D-glucose. The internal solution of the glass pipettes contained (in mM) 100 cesium gluconate, 0.2 EGTA, 0.5 MgCl₂, 2 ATP, 0.3 GTP, and 40 HEPES. The pH of internal solution was normalized to 7.2 with cesium hydroxide and diluted to a trace osmotic deficit in comparison to ACSF (˜300 mOsm). All analysis of recordings was performed manually using MiniAnalysis (Synaptosoft Inc., Fort Lee, N.J.). Minimum parameters were set at greater than 1 min stable recording, and event amplitude greater than 2 pA. A mEPSC event was identified by distinct fast-rising depolarization and slow-decaying repolarization. Combined individual events were used to form relative cumulative frequency curves, whereas the means of all events from individual recordings were treated as single samples for further statistical analysis.

### Image Analysis of Live Neuronal Cultures

Transfected cells were continually bathed in neurobasal media and were passively perfused with medical-grade 95% O₂-5% CO₂. Micrographs were taken on a Nikon epifluorescent inverted microscope with 60× oil lens with a computerized focus motor at DIV 21-23. All digital images were processed using METAMORPH Imaging System (Universal Imaging Corporation, Molecular Devices, San Jose, Calif.). Images were taken as 15 plane stacks at 0.5 micron increments, processed by deconvolution to the nearest planes, and averaged against other stacked images. A dendritic spine was defined as having an expanded head diameter, greater than 50% larger in diameter than the neck. The number of spines per neuron were counted and normalized to a 100 m length of dendritic shaft.

### Pharmacology

Roscovitine and CHIR99021 were purchased from Sigma-Aldrich (St. Louis, Mo.) and were diluted in DMSO to four 1000× concentration aliquots for ultimate concentrations in neurobasal medium of 0.05, 0.5, 5 and 10 M. Primary cultured rat hippocampal neurons transfected with dsRed and eGFP-P301L-tau were treated with one of four 1000× aliquots or DMSO vehicle on DIV 20-22. Treated cells were incubated for 24 hours prior to imaging on DIV 21-23. Cell death was visually assessed under differential interference contrast (DIC) for decreased cell density, lost soma adhesion, gross qualitative neurite retraction. If evidence of cell death was observed under DIC, cells were fixed in 4% sucrose and 4% paraformaldehyde in PBS and stained with 300 nM DAPI in PBS. DAPI stained cells were analyzed under fluorescent microscopy for nuclear pyknosis and karyorrhexis; dsRed expressing cells were analyzed for spine loss. If no cell death was evident under DIC, live fluorescent images were acquired and analyzed as above.

### Statistics

All statistics were performed in Prism 6 (Graphpad Software, San Diego, Calif.). One- and two-way ANOVA was used for univariate and two-variable analysis respectively. If ANOVA revealed significant variance between all groups, post-hoc analysis was performed using Bonferroni analysis adjusted for multiple groups. Univariate cumulative frequency distributions were compared using the unmodified Kolmogorov-Smirnov goodness of fit test. For all, statistical significance was set for α=0.05.

### Results and Discussion

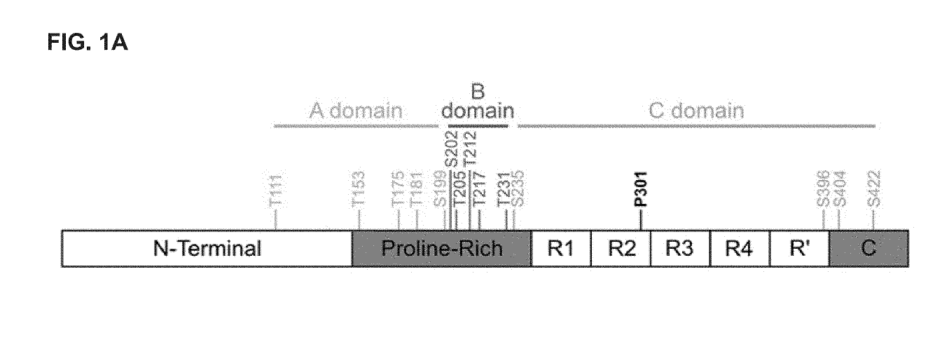

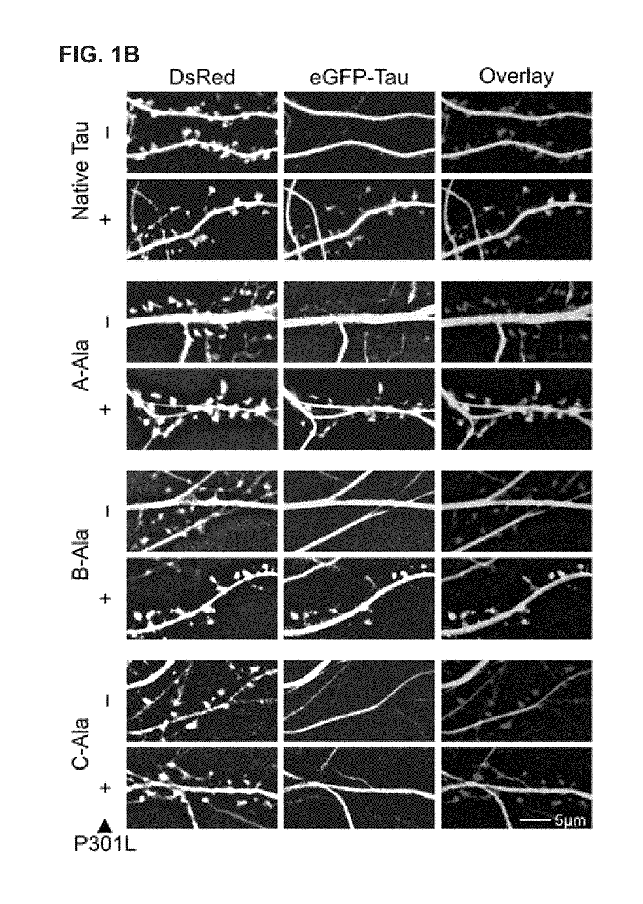

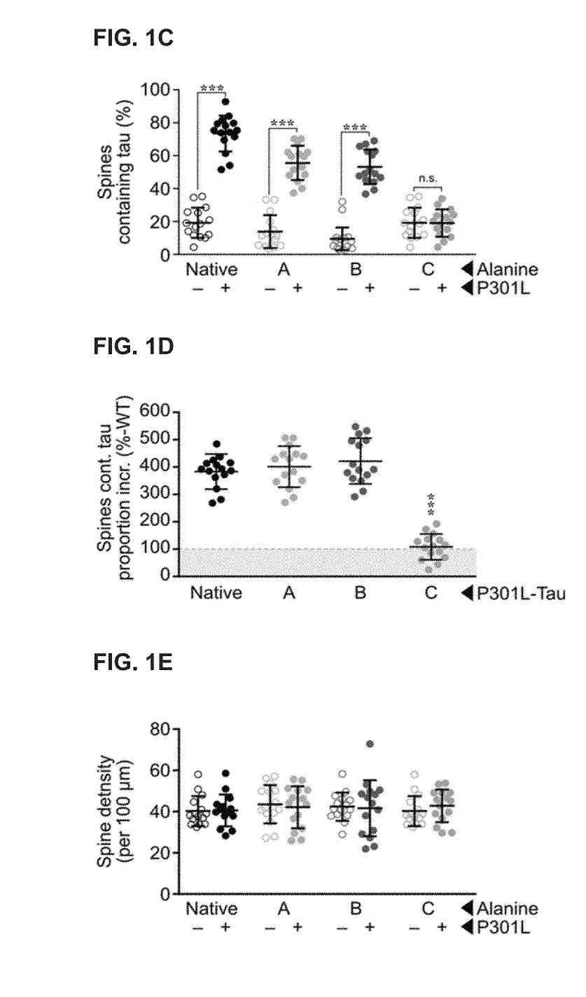

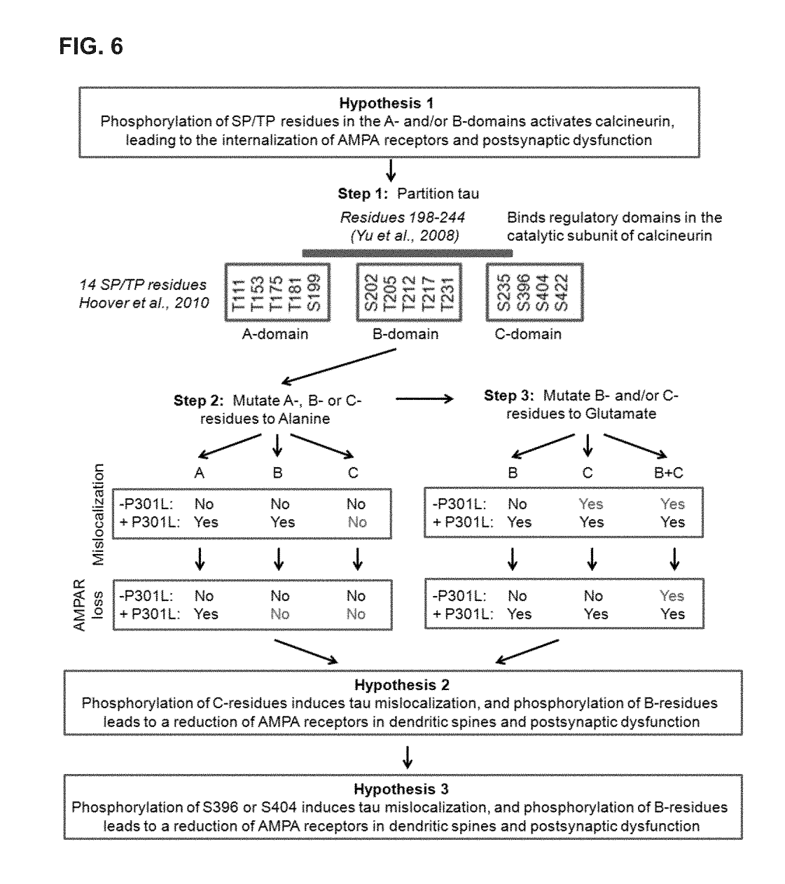

To test if differential phosphorylation of distinct tau domains impairs postsynaptic function, three domains (referred as A-, B- and C-domains) of tau were formulated in a semi-random fashion, each containing clusters of four or five SP/TP residues (FIG. 1A; see also FIG. 6). T111, T153, T175, T181 and S199 constitute the A-residues in the A-domain; S202, T205, T212, T217 and T231 constitute the B-residues in the B-domain; and S235, S396, S404 and S422 constitute the C-residues in the C-domain. To determine the differential effects of phosphorylation within each domain, SP/TP residues in each domain were systematically mutated to alanine (Ala) to block phosphorylation and to glutamate (Glu) to mimic phosphorylation, and the effects of the variant and native tau proteins were compared.

As used in this Example, a “P301L mutant” refers to tau with the P301L mutation, a mutation linked to frontotemporal dementia with parkinsonism linked to chromosome 17, and “wild type” refers to tau without the P301L mutation. As used herein, “variant” refers to tau with SP/TP substitutions, and “native” refers to tau without SP/TP substitutions.

To identify the phosphorylation residues that regulate the mislocalization of tau, the effect on the subcellular distribution of tau of blocking phosphorylation in each of the three domains was tested. At 7-10 days in vitro, dsRed (to visualize cellular morphology) and P301L mutant or wild type eGFP-tau constructs with alanine substitutions in the A-domain (A-Ala), B-domain (B-Ala) or C-domain (C-Ala) were co-expressed in cultured rat hippocampal neurons. At 21 days in vitro (DIV), the dendrites of live neurons were photographed and the percentage of spines containing eGFP-tau was determined. eGFP-tau was found to be distributed throughout the dendritic shaft in all conditions, and significantly more eGFP-containing spines were observed in neurons expressing P301L mutant eGFP-tau (FIG. 1B-FIG. 1C). Neurons expressing the A-Ala and B-Ala variants of P301L mutant eGFP-tau showed slight reductions in the percentage of eGFP-tau-containing spines (F=96.57, P<0.001) (FIG. 1B-FIG. 1C), but these changes were not significant when the data were normalized to their respective wild type tau control groups (FIG. 1D). Interestingly, in neurons expressing the C-Ala variant of P301L mutant eGFP-tau, the percentage of eGFP-containing spines dropped dramatically, to that of neurons expressing wild type eGFP-tau (FIG. 1B-FIG. 1D). Thus, blocking phosphorylation of SP/TP residues in the C-domain, but not the A- and B-domains, prevented P301L-induced mislocalization to dendritic spines. No change in spine density among the various tau species was observed (FIG. 1E), indicating no overt synaptotoxicity associated with mislocalization over the period observed in this paradigm.

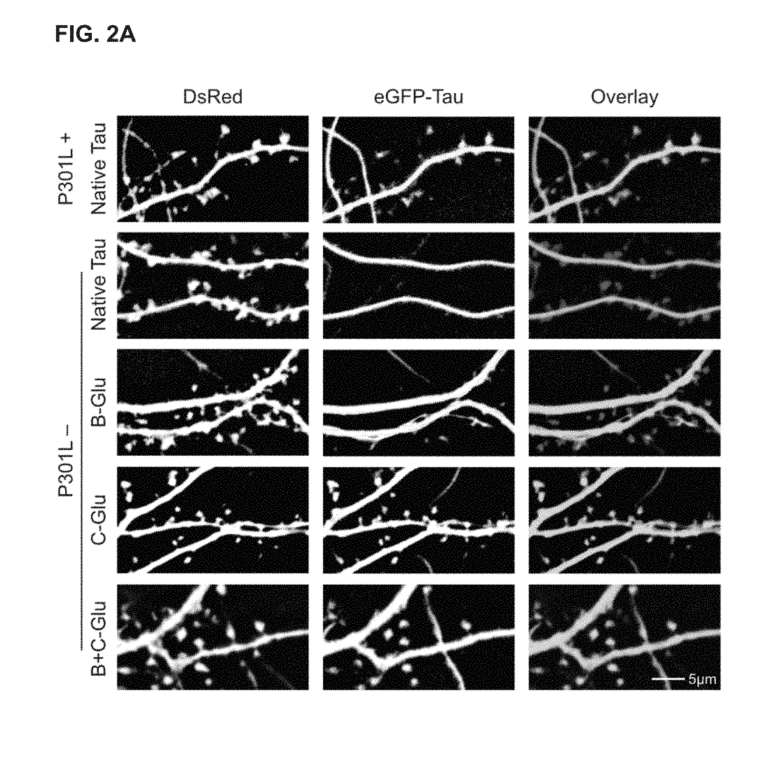

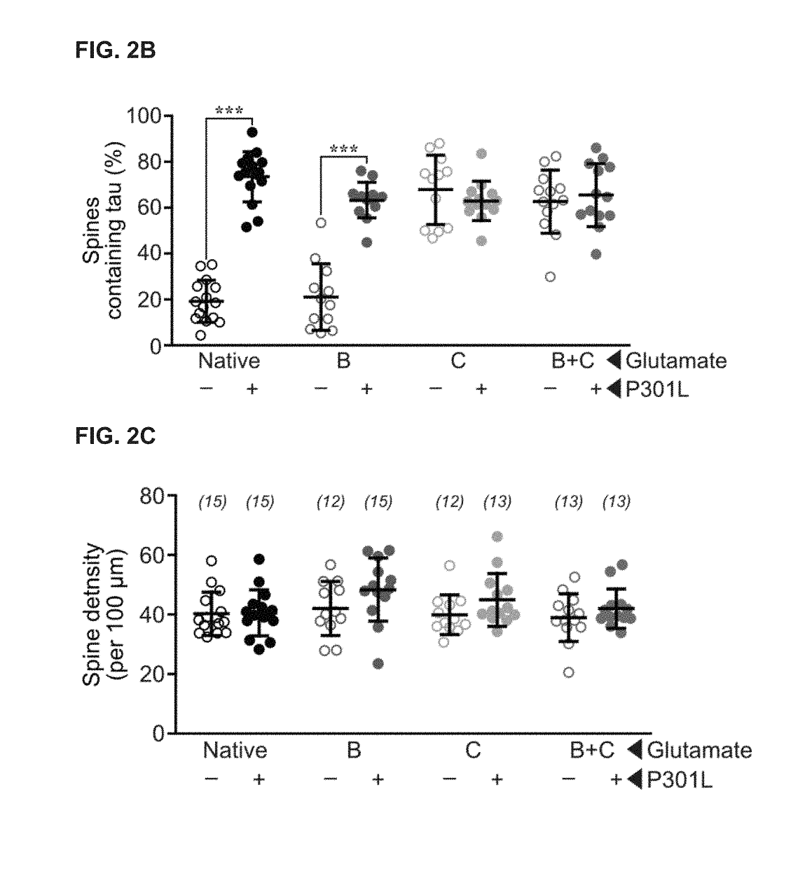

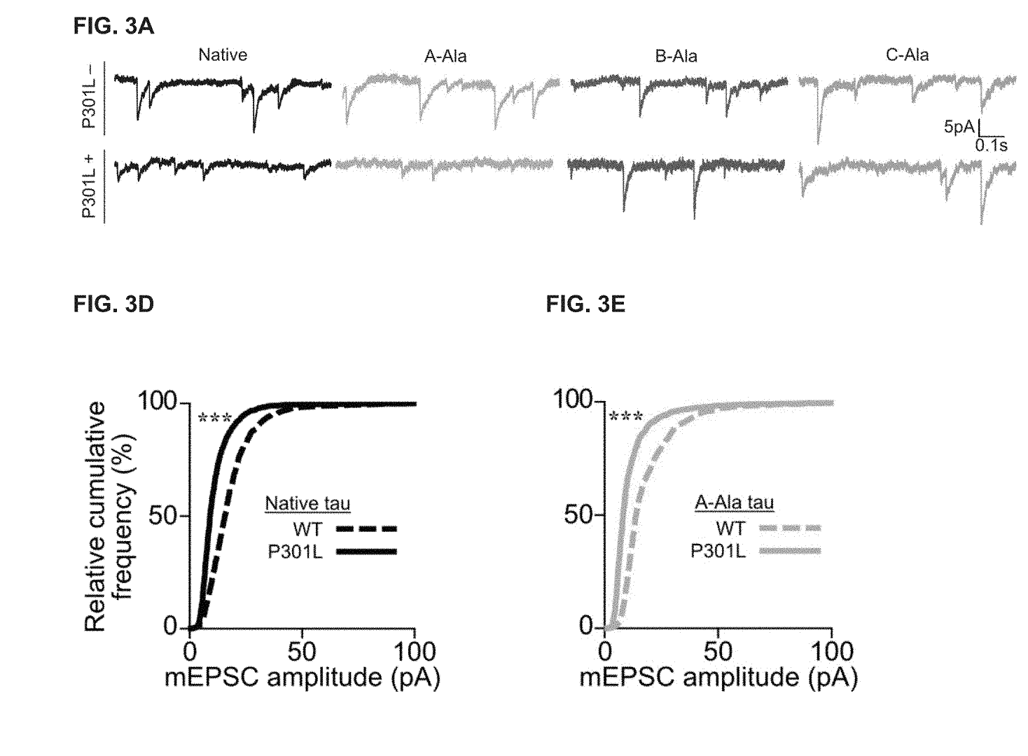

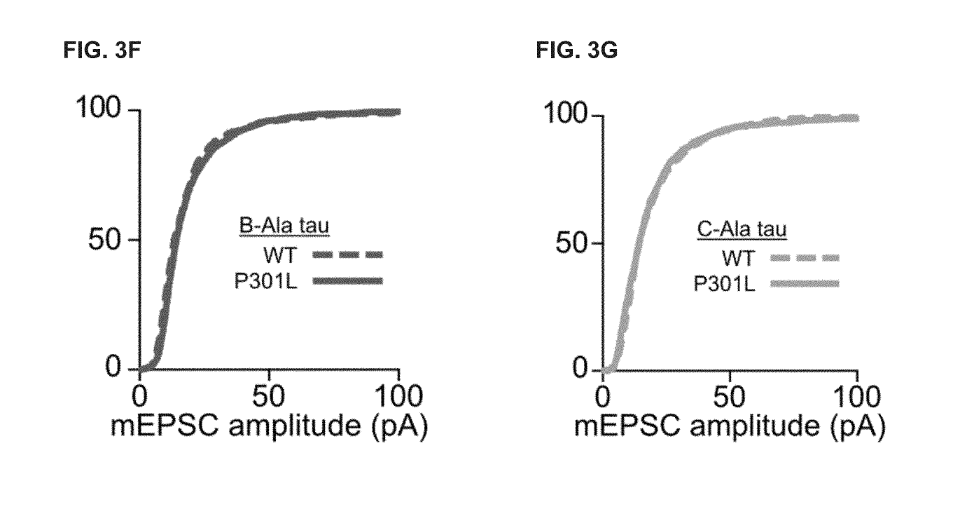

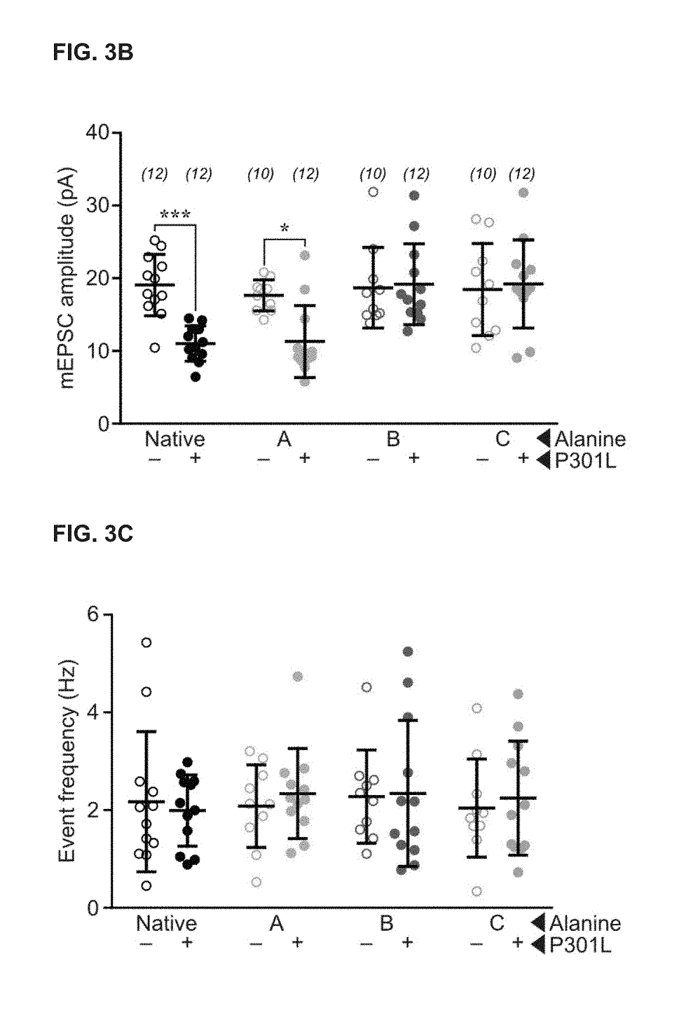

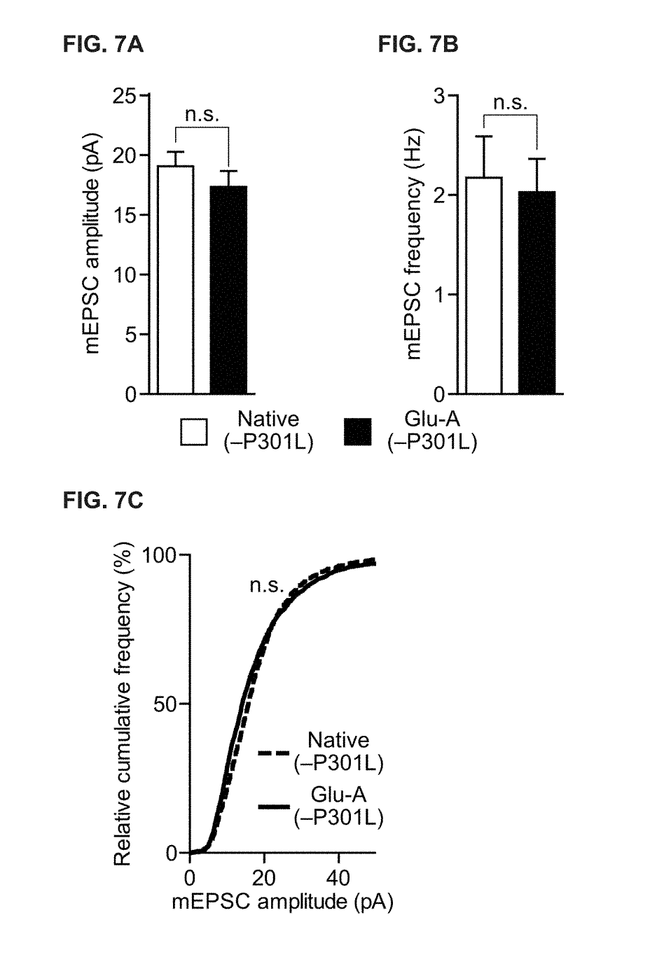

Additional support for this conclusion was obtained by evaluating the effects of differential phosphorylation in each domain using tau variants with glutamate mutations, which mimic phosphorylation by increasing the negative charge. Because pseudophosphorylation of the A-residues is neither necessary nor sufficient to mediate tau-induced deficits (FIG. 3 and FIG. 7), the effects of pseudophosphorylation of the B-residues and C-residues were characterized. At 7-10 days in vitro, dsRed and P301L mutant or wild-type eGFP-tau constructs with glutamate substitutions in the B-domain (B-Glu) or C-domain (C-Glu) were co-expressed in cultured rat hippocampal neurons (FIG. 2). At 21 days in vitro, the dendrites of live neurons were photographed and the percentage of spines containing eGFP was determined (FIG. 2A-FIG. 2B). In tau variants that contain the P301L mutation, neurons expressing native, P301L mutant eGFP-tau and all three phosphomimetic variants of P301L mutant eGFP-tau showed a high percentage of eGFP-containing spines. In the absence of the P301L mutation, neurons expressing native wild-type eGFP-tau showed a low percentage of eGFP-containing spines. Interestingly, expressing the C-Glu variant of wild type eGFP-tau led to dramatically increased percentages of eGFP-containing spines that were comparable to expressing P301L mutant eGFP-tau (FIG. 2B). In contrast, expressing the B-Glu variant of wild type eGFP-tau did not lead to an increase in eGFP-containing spines. These results support the conclusion that the phosphorylation of one or more C-residues, but not B-residues, leads to the mislocalization of wild type tau to an extent that is equivalent to P301L mutant tau.

The results described above demonstrate a role for phosphorylation on the mislocalization of tau to the dendritic spine, a glutamatergic postsynaptic compartment. Next, the role of phosphorylation in postsynaptic function was evaluated by testing the effect of blocking phosphorylation on AMPA receptor function. A-Ala, B-Ala and C-Ala variants of P301L mutant and wild type eGFP-tau were expressed in cultured rat hippocampal neurons, and whole-cell, patch-clamp electrophysiology was performed to record glutamatergic mini-excitatory postsynaptic currents (mEPSCs). In neurons expressing P301L mutant eGFP-tau, mEPSCs with smaller amplitudes (black lines and symbols, FIG. 3A, FIG. 3B, and FIG. 3D) and normal frequencies (FIG. 3C) were observed. The preservation of mEPSC frequencies, indicating normal presynaptic function, probably results from the very low transfection rates in these experimental system (˜1%), making it unlikely that a patched cell would be innervated by a neuron expressing P301L mutant eGFP-tau. Alanine substitutions in the A-, B- and C-domains produced different effects on postsynaptic dysfunction caused by the P301L mutation. The mEPSCs in neurons expressing the A-Ala variant of P301L mutant eGFP-tau remained abnormally small (orange lines and symbols, FIG. 3A, FIG. 3B, and FIG. 3E), indicating that postsynaptic function was not affected by phosphorylation in the A-domain. However, both B-Ala and C-Ala substitutions restored mEPSCs (blue and pink lines and symbols, FIGS. 3A, 3B, 3F, 3G). Since the high percentage of eGFP-containing dendritic spines in neurons expressing the B-Ala variant of P301L mutant eGFP-tau is indicative of mislocalization in neurons (FIG. 1C), the normal mEPSCs in these neurons was surprising, and it shows that tau mislocalization alone is not sufficient to induce postsynaptic dysfunction. These results suggest that postsynaptic dysfunction is contingent on mislocalization, which depends on C-domain phosphorylation, as well as on additional phosphorylation in the B-domain.

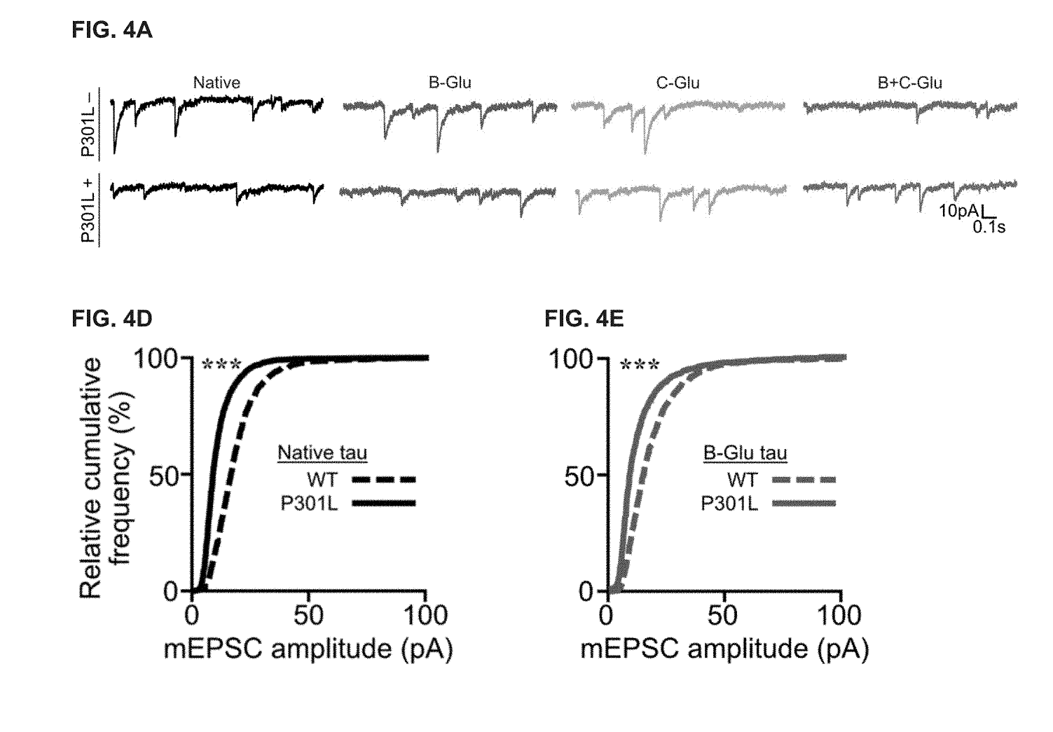

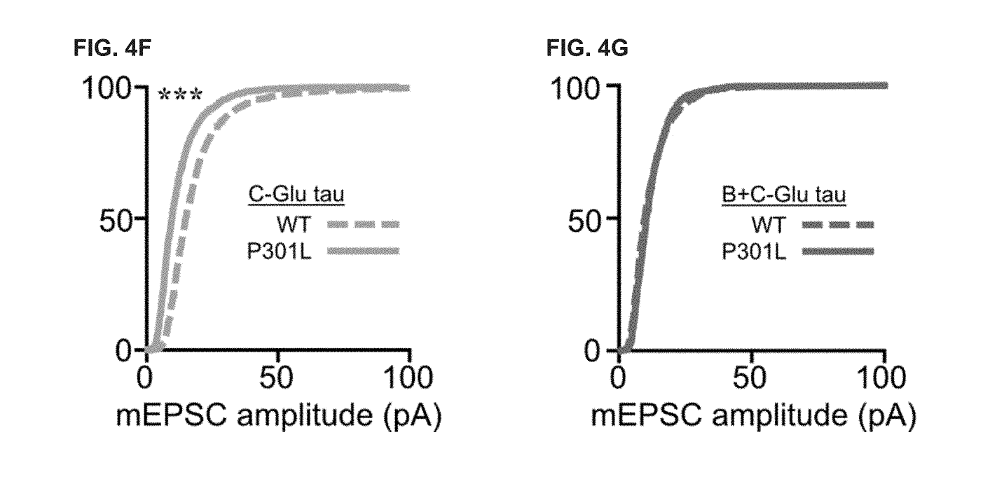

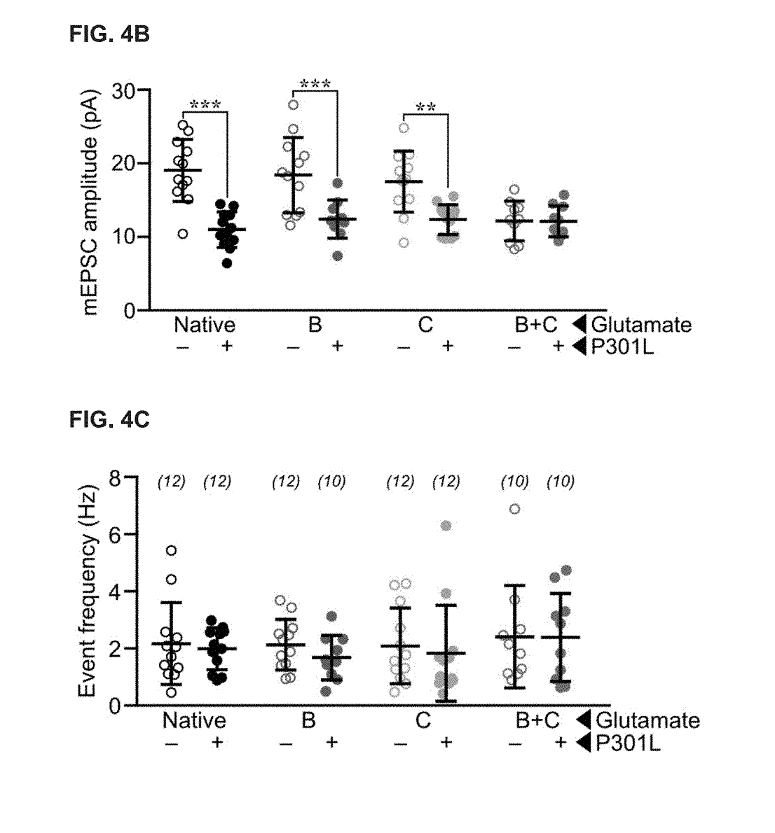

To further test the hypothesis that the phosphorylation within the B- and C-domains collaborates to disrupt postsynaptic function, the effects of B-Glu, C-Glu and B+C-Glu on tau-induced glutamatergic postsynaptic function were examined by measuring mEPSCs in cultured rat hippocampal neurons expressing P301L mutant or wild type eGFP-tau. As expected, neurons expressing P301L mutant eGFP-tau showed reductions in mEPSC amplitudes, irrespective of phosphomimetic mutations (FIGS. 4A, 4B, 4D-4G). In neurons expressing wild type eGFP-tau, however, neither B-Glu nor C-Glu alone altered mEPSCs (blue and pink lines and symbols, FIGS. 4A, 4B, 4D-4G)). Interestingly, mEPSC amplitudes were greatly reduced in neurons expressing the B+C-Glu variant of wild type eGFP-tau (green lines and symbols, FIG. 4A, FIG. 4B, and FIG. 4G), indicating that postsynaptic dysfunction depends on phosphorylation in both domains. Taken together with the results of the phospho-blocking experiments, these results indicate that postsynaptic dysfunction occurs through a coordinate series of events entailing first, mislocalization to spines that depends on phosphorylation in the C-domain and second, a weakening of AMPA receptor-mediated postsynaptic responses that depends on phosphorylation in the B-domain

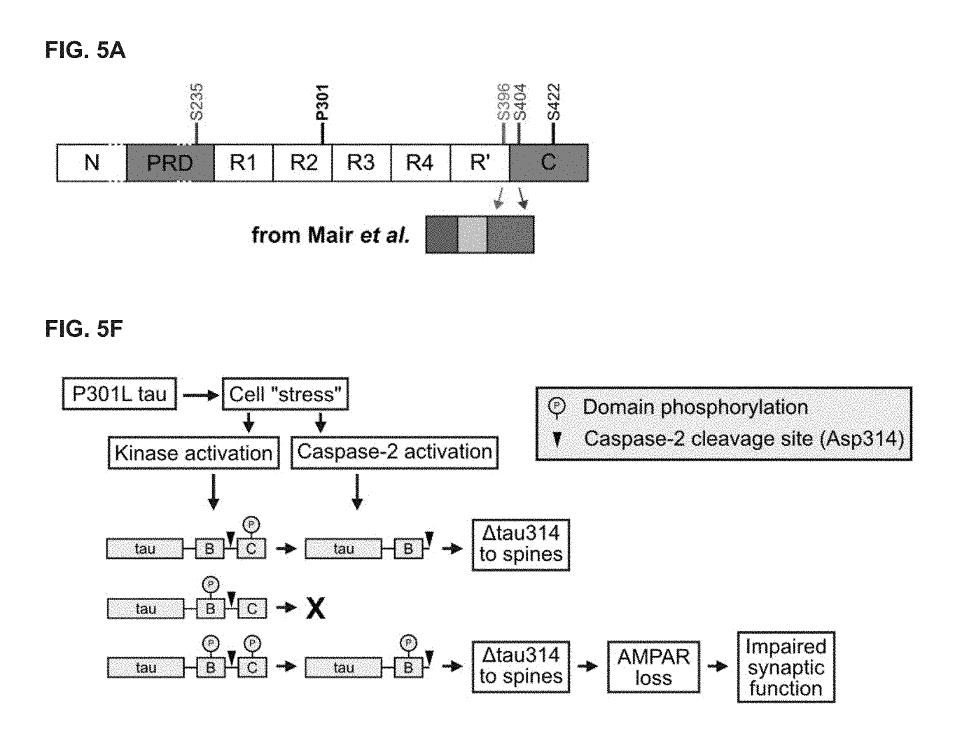

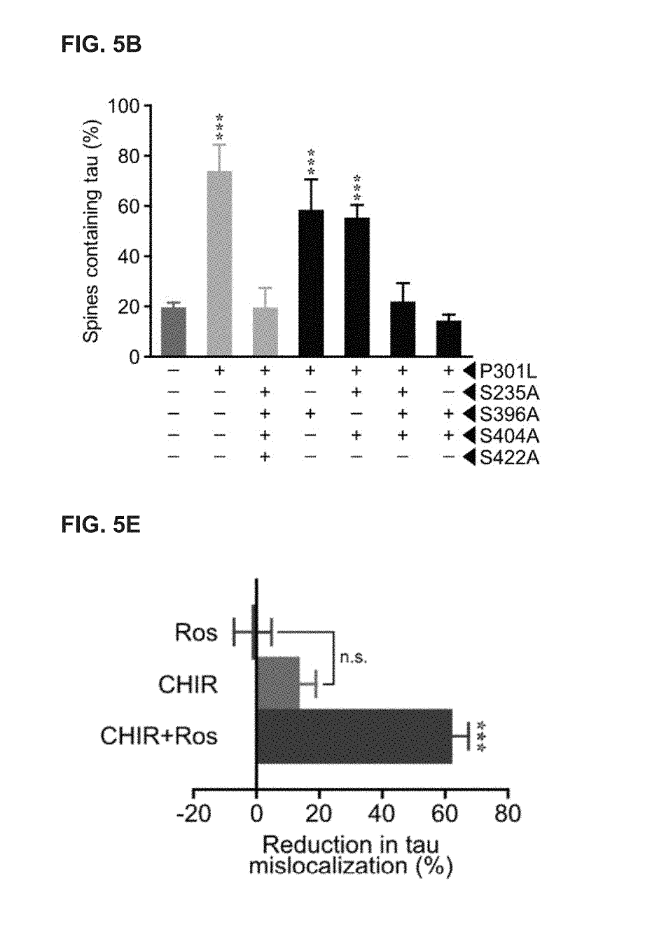

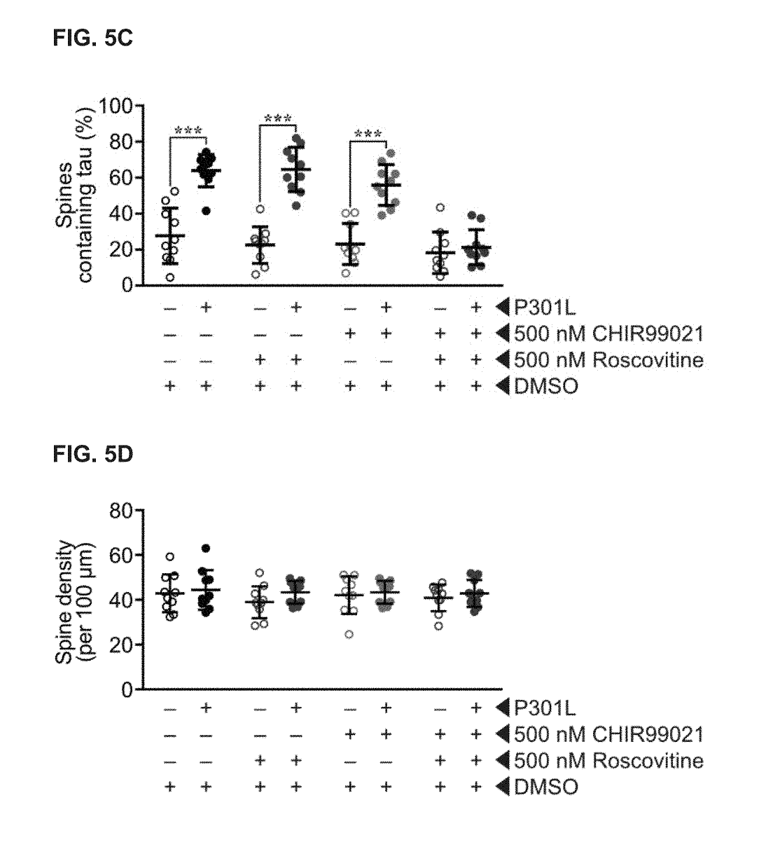

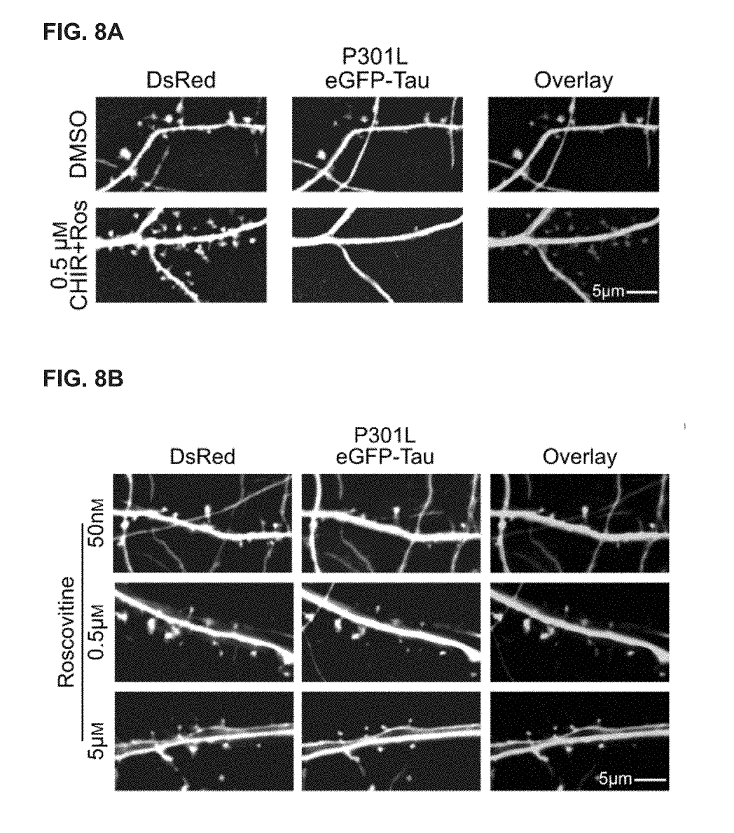

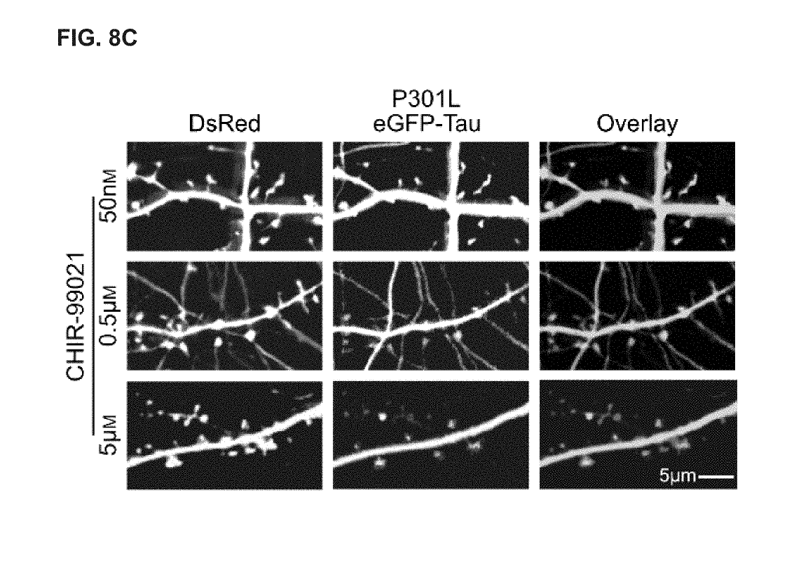

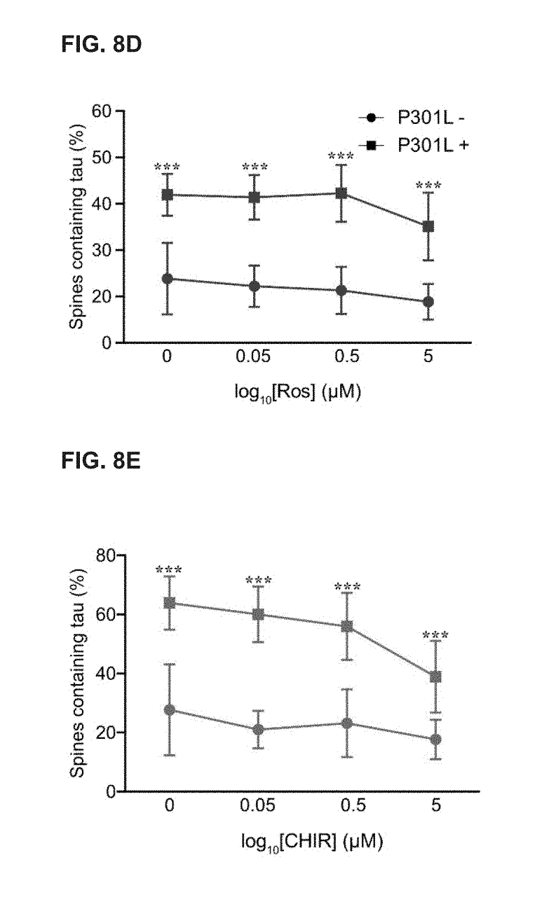

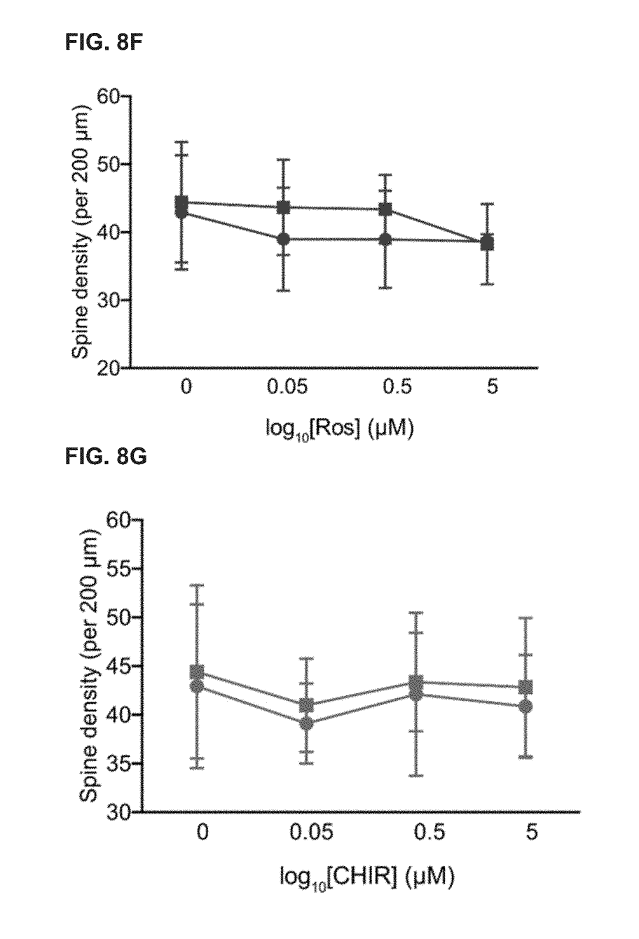

To refine the identification of C-residues responsible for mislocalization, phosphorylation of specific SP/TP residues in the C-domain of P301L mutant eGFP-tau was blocked by mutating those residues to alanine (FIG. 5A, FIG. 5B). Importantly, P301L mutant tau-induced mislocalization was blocked to the same extent with S396A:S404A as with S235A:S396A:S404A:S422A, suggesting that simultaneous blockade of the phosphorylation of only two residues, S396 and S404, is sufficient to ameliorate the tau-induced abnormalities. S235A:S396A:S404A also blocked tau mislocalization, excluding a role for S422 phosphorylation in this cellular change. Blocking S235 and either S396 or S404 reduced the percentage of eGFP-containing spines slightly from approximately 70% to approximately 58% but did not abolish mislocalization. Taken altogether, these results indicate that phosphorylation at either S396 or S404 is sufficient to induce the maximum degree of mislocalization. To confirm this conclusion, kinase inhibitors were used to block phosphorylation. Based on previously reported 2D-phosphopeptide mapping of purified cell lysates (Kimura et al. 2014 Front. Mol. Neurosci. 7, 1-10; Illenberger et al. 1998 Mol. Biol. Cell 9, 1495-1512; Tenreiro et al. 2014 Front. Mol. Neurosci. 7, 1-30; Grueninger et al. 2011 Mol. Cell. Biochem. 357, 199-207), S404 is phosphorylated by cyclin-dependent kinase 5 (cdk5) and S396 is phosphorylated by glycogen synthetase kinase 3β (gsk3β) (illustrated in FIG. 5A). Treating neurons expressing P301L mutant eGFP-tau with 500 nM chir99021, a gsk3β inhibitor, and 500 nM roscovitine, a cdk5 inhibitor, in combination reduced the percentage of eGFP-containing spines to that of wild type eGFP-tau (FIG. 5C, FIG. 5E). However, neither drug alone, at concentrations up to 5 μM, lowered the percentage of eGFP-containing spines to control levels (FIG. 5C, FIG. 5E and FIG. 8). Concentrations of chir99021 above 5 μM killed the neurons. These results indicate that the inhibition of both kinases is necessary to suppress tau mislocalization, suggesting that tau phosphorylation by either gsk3β or cdk5 can activate a redundant signaling cascade that leads to synaptic deficits. These pharmacological results strongly support mutational analysis showing that phosphorylation of either S396 or S404 is sufficient to promote tau mislocalization to dendritic spines.

There are 85 putative phosphorylation residues in tau, which vary in their extent of phosphorylation. Using a mass-spectrometry-based assay to measure the stoichiometry of phosphorylated residues in soluble wild type tau expressed in Sf9 insect cells and human neuronal iPSCs, the most frequently phosphorylated SP/TP residues were found to be S199, S202, T205, T212, T217, T231, S235, S396 and S404. Specifically, ˜85% of the wild-type-tryptic fragments containing S396 and S404 were modified (expressed as ˜15% unmodified), indicating that one or both residues are phosphorylated in ˜85% of wild type tau molecules expressed. If the stoichiometry of phosphorylation at these two residues is as high under “normal” physiological conditions, then most wild type tau proteins would be mislocalized in dendritic spines, which contradicts previous findings. This discrepancy suggests that the stoichiometry of phosphorylation in primary neurons and the brain may differ from that in the insect and iPSC culture paradigms, or that one or more “bottlenecks” or rate-limiting steps exist in the pathway leading to tau mislocalization. For example, it has been previously reported that the truncation of tau at D314 is also required for tau mislocalization (Zhao et al. 2016 Nat. Med. 22, 1268-1276).

This Example shows that postsynaptic dysfunction is the result of a coordinated progression of differential phosphorylation and cleavage, as depicted in the conceptual model of FIG. 5F. In cultured neurons, preventing the phosphorylation of both S396 and S404 or blocking proteolytic cleavage at D314 reduced the mislocalization of tau to dendritic spines. Blocking mislocalization or the phosphorylation of one or more B-residues (S202, T205, T212, T217, T231) prevented the reduction of mEPSC amplitudes. Through systematic mechanistic investigations, it was deduced that the phosphorylation of either S396 or S404 in combination with cleavage at D314 promotes the mislocalization of tau to dendritic spines, and that the phosphorylation of one or more B-residues reduces the levels of AMPA receptors in the postsynaptic membrane.

The exact upstream factors causing cellular stress leading to the pathological activation of proteases and kinases are unknown. The unfolded protein response that is activated by endoplasmic reticulum stress (ER stress) (Su et al. 2016 Nat. Cell Biol. 18, 527-539) increases phosphorylation of the S202 and S205 residues in the B-domain (Kim et al. 2017 PLoS Genet. 13, 1-22). ER stress activates gsk3β (Liu et al. 2016 Mol. Neurobiol. 35, 983-994) and caspase-2 (Uchibayashi et al. 2011 J. Neurosci. Res. 89, 1783-1794).

The dysregulation of cdk5, which phosphorylates the C-site S404 of tau, and gsk3β, which phosphorylates the C-site S396 of tau, have been previously implicated in the pathogenesis of Alzheimer's disease. A combination of gsk3β and cdk5 inhibitors was needed to block tau-mediated synaptic changes, offering a potential explanation for the failure of tideglusib, a gsk3β inhibitor, in a recent clinical trial (Lovestone et al. 2015 J. Alzheimers Dis. 45, 75-88).

It may be of interest to delineate the downstream events leading to a reduction in postsynaptic AMPA receptors. One possibility is the internalization of GluA1 subunits of AMPA receptors through dephosphorylation by calcineurin, which interacts with a segment in the proline-rich domain (aa 198-244) encompassing the B-domain (S202-T231; FIG. 1A and FIG. 6). In line with this, the calcineurin inhibitor FK-506 prevents tau-induced loss of AMPA receptors in dendritic spines by blocking the dephosphorylation of GluA1 (Miller et al. 2014 Eur. J. Neurosci. 39, 1214-1224; Miller et al. 2012 Mol. Pharmacol. 82, 333-343; Kam et al. 2010 J. Neurosci. 30, 15304-15316).

### Example 2

As shown in Example 1, phosphorylation of the C-domain drives tau-missorting whereas the B-domain drives subsequent loss of AMPA receptors (AMPARs) caused by tau. Without wishing to be bound by theory, it is believed that that Alzheimer's disease (AD) and/or traumatic brain injury (TBI) activate glycogen synthase kinase 3 beta (gsk3β) and cyclin-dependent kinase 5 (cdk5) which phosphorylate the C-domain of tau, driving tau to spines, resulting in further phosphorylation of the B-domain, causing loss of AMPARs (see FIG. 5F for a hypothetical signaling cascade).

### Peptides

Two peptides were synthesized to block the initial step of the cellular cascade that leads to tau-mediated synaptic deficits:
H2N-GRKKRRQRRRPQDHGAEIVYKSPVVSGDTSPRHLSNVS ST-OH (Peptide 1; wild-type form; also referred to herein as WT peptide, SEQ ID NO: 1) and
H2N-GRKKRRQRRRPQDHGAEIVYKAPVVSGDTAPRHLSNVS ST-OH (Peptide 2; blocking form, with the AP mutation; also referred to herein as AP peptide, SEQ ID NO:2).

An additional peptide was synthesized and tested but did not block the cellular cascade that leads to tau-mediated synaptic deficits:
H2N-GRKKRRQRRRPQDHGAEIVYKEPVVSGDTEPRHLSNVS ST-OH (Peptide 3; also referred to herein as EP Peptide, SEQ ID NO:3)
   The HIV Trans-Activator of Transcription (TAT) domain sequence increases cell permeability of the peptides. Peptide 1 includes the sequence of wild-type human tau and includes S396 and S404. Peptide 2 includes a sequence of human tau that includes mutations (serine to alanine) at positions S396 and S404 (S396A and S404A), mutations that block the phosphorylation of these two sites. Peptide 3 includes the same sequence but with mutations to glutamic acid at positions S396 and S404 (S396E and S404E).

### Effects of the Newly Synthesized Peptides on Two Tauopathy Models

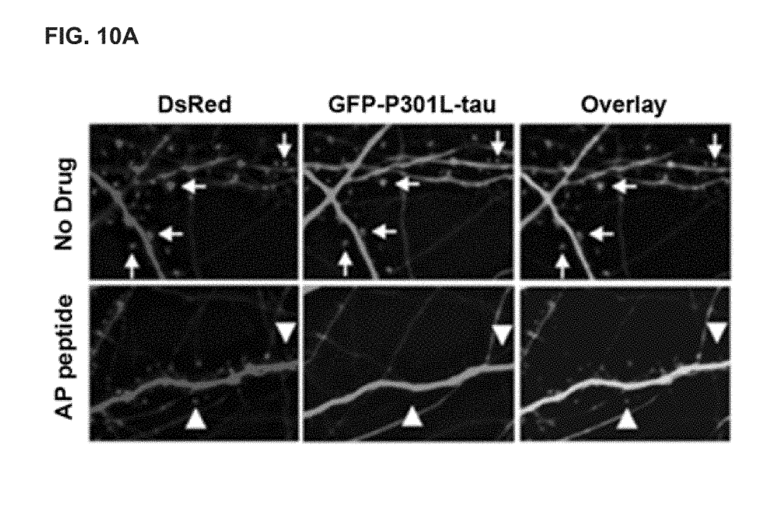

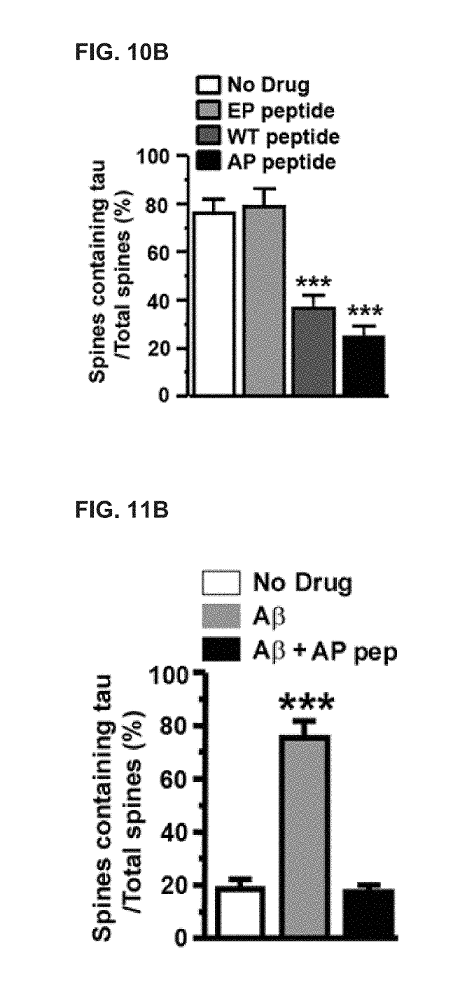

The majority (>60%) of frontotemporal dementia with parkinsonism-17 (FTDP-17) is caused by three mutations: P301L/S, N279K and “10+16”. Cells and animals expressing a P301L mutant tau are frequently used as tauopathy models (Snowden et al. 2002 Br J Psychiatry 180:140-143). In neurons expressing P301L mutant tau proteins, both WT peptide and AP peptide blocked tau mis-sorting, and the AP peptide was observed to have a stronger effect. In contrast, the EP peptide did not block tau mis-sorting. Results are shown in FIG. 10.

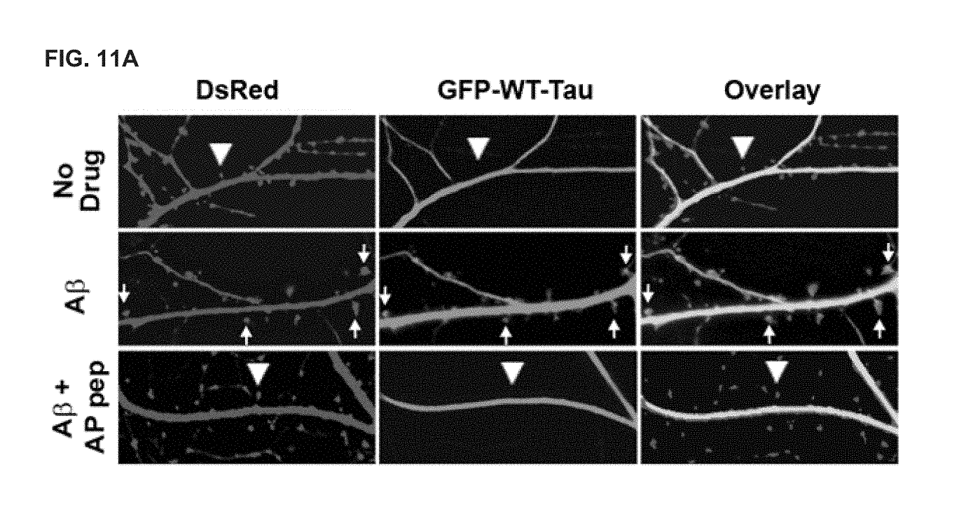

In another tauopathy model, the AP peptide was observed to block tau mislocalization caused by Aβ oligomers, which are believed to be the key initiator of neural deficits in AD. Results are shown in FIG. 11.

### AP Peptide Blocks Tau Mis-Sorting in Mechanically Injured Neurons

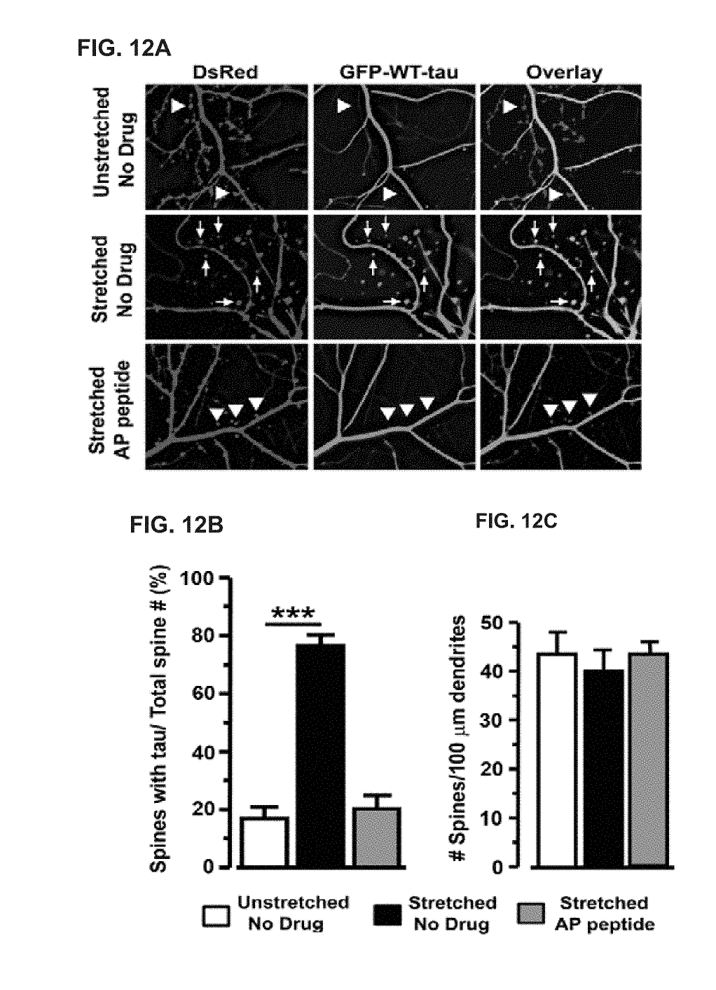

In a tauopathy model for traumatic brain injuries (TBI) based on the model of Hemphill et al. 2011 PLoS One. 6(7):e2289), mechanical injuries to neurons induce tau missorting to dendritic spines (FIG. 12). The methods of Hemphill et al. were modified such that plasmids encoding GFP-tagged tau were introduced into neurons, allowing detection of tau abnormalities as well as tau-mediated synaptic deficits. Briefly, neurons were plated onto medical grade silicone elastomer membranes (0.010 inch NRV, Specialty Manufacturing, Inc., Saginaw, Mich.) and glued inside a reducing well. Each sample was loaded into a custom-made High Speed Stretching (HSS) device which used a high precision linear motor (Model P01-23×80F-HP, LinMot USA, Inc., Elkhorn, Wis.) to displace the brackets and strain the elastomer sheet to a desired magnitude at a rate of 1% per millisecond in one horizontal dimension. The presence of AP peptide (1 μM) blocked tau missorting to dendritic spines caused by a series of mechanical strains.

### Example 3—A53T Mutant Alpha-Synuclein Induces Tau Dependent Postsynaptic Impairment Independent of Neurodegenerative Changes

This Example shows that A53T α-synuclein, which is associated with familial Parkinson's disease, induces phosphorylation-dependent tau mislocalization to dendritic spines and associated postsynaptic deficits.

Abnormalities in α-synuclein are implicated in the pathogenesis of Parkinson's disease. Because α-synuclein is highly concentrated within presynaptic terminals, presynaptic dysfunction has been proposed as a potential pathogenic mechanism. As further described in this Example, synaptic activity in hippocampal slices and cultured hippocampal neurons from transgenic mice expressing human wild-type, A53T, and A30P α-synuclein was analyzed. Increased α-synuclein expression was found to lead to decreased spontaneous synaptic vesicle release regardless of genotype. However, only those neurons expressing A53T α-synuclein were found to exhibit postsynaptic dysfunction including decreased miniature postsynaptic current amplitude and decreased AMPA to NMDA receptor current ratio. Mechanistically, postsynaptic dysfunction requires GSK3β-mediated tau phosphorylation, tau mislocalization to dendritic spines, and calcineurin-dependent AMPA receptor internalization. a novel, functional role for tau: mediating the effects of α-synuclein on postsynaptic signaling. This tau-mediated signaling cascade may contribute to the pathogenesis of dementia in A53T α-synuclein-linked familial Parkinson's disease cases as well as some subgroups of Parkinson's disease cases with extensive tau pathology.

### Introduction

Parkinson's disease (PD) is the second most common late-onset neurodegenerative disease. It is characterized by both motor symptoms and the convergence of alpha-synuclein (αS), tau, and amyloid-β pathology. Sporadic PD is clinically heterogeneous. Genetic abnormalities including αS gene (SNCA) amplification, as well as A53T, A30P, and E46K αS point mutations are linked to familial PD. These inherited forms of PD are also heterogeneous. Each features a different time of onset, clinical presentation and histopathology (Petrucci et al. 2015 Parkinsonism Relat. Disord. 22 Supl: S16-22). Particularly, tau and αS pathologies frequently coexist in the Contursi kindred who carry the A53T αS point mutation (Duda et al. 2002 Acta Neuropathol. 104(1):7-11). This Example describes the exploration of whether A53T mutant αS activates additional signaling pathways that are distinct from those activated by wild-type (WT) αS and other mutants. αS is a cytosolic protein that is enriched in the presynaptic terminals of neurons and can associate with the plasma membrane. To identify the cellular mechanisms underlying the clinical and pathological diversity of PD, changes in the synaptic function of neurons expressing multiple αS variants were compared. This Example describes the finding that A53T αS induces postsynaptic deficits that require GSK3β-dependent tau missorting to dendritic spines and calcineurin-dependent loss of postsynaptic surface AMPA receptors.

### Materials and Methods

All common reagents used in this Example were purchased from Sigma Aldrich (St. Louis, Mo.) unless otherwise noted.

### Animals

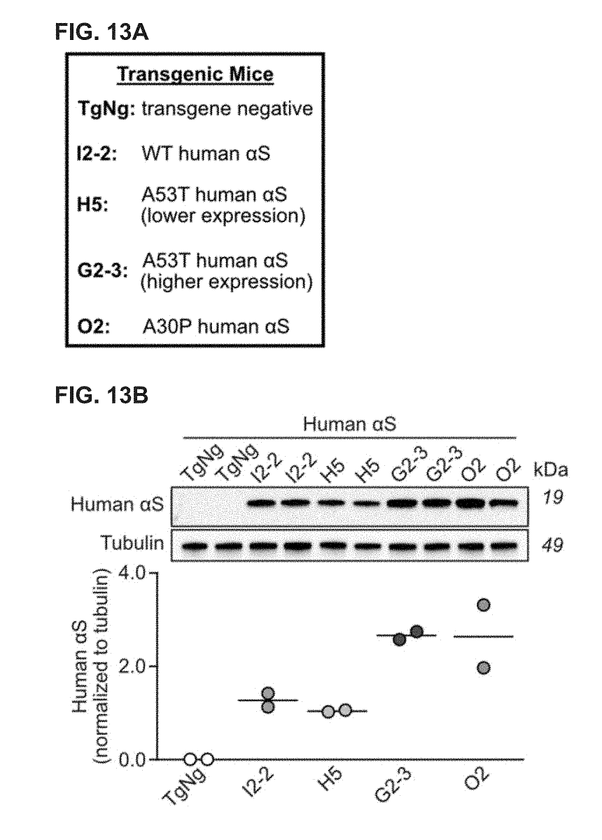

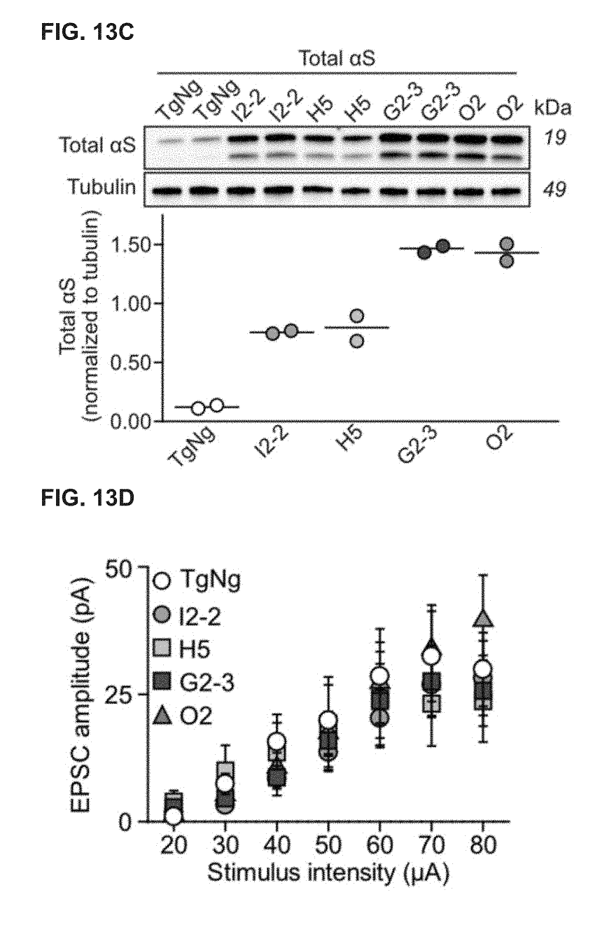

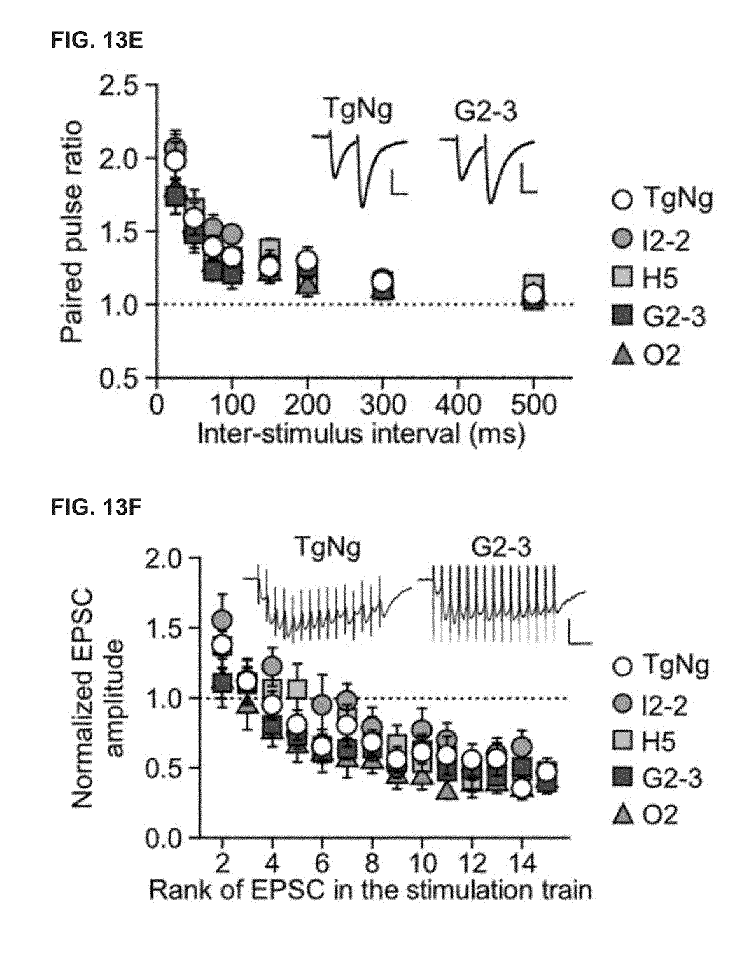

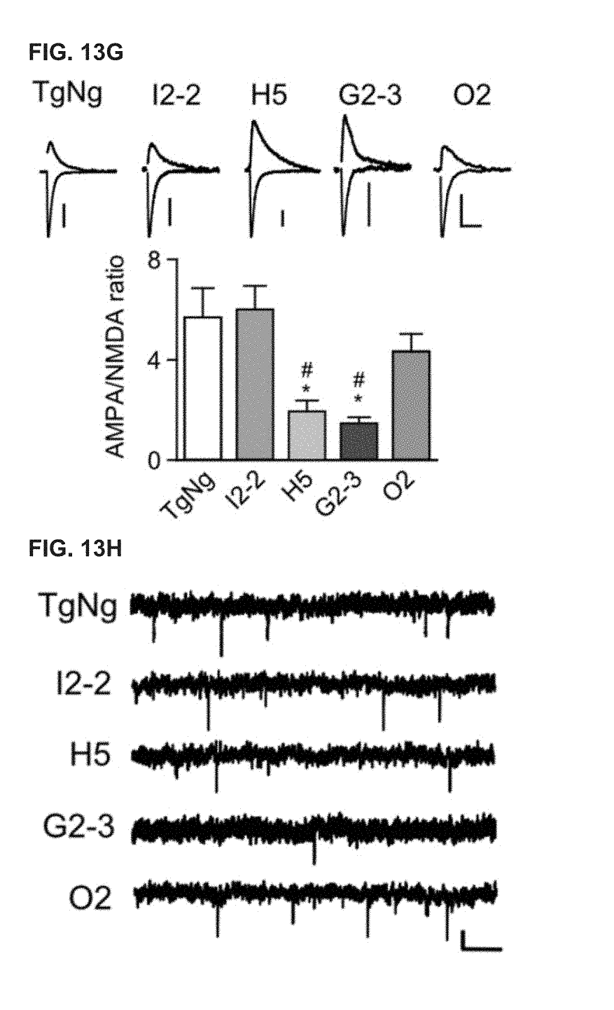

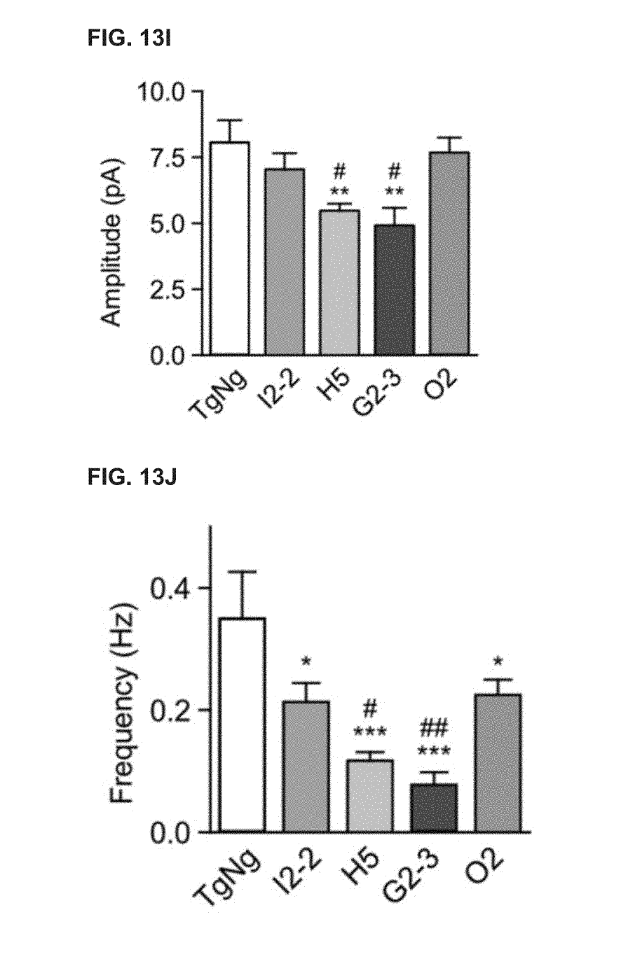

The four transgenic mouse lines used in the present study are listed in FIG. 13A. The method for the generation of transgenic (Tg) mice that express human WT (line 12-2), A53T mutant (lines G2-3 and H5), and A30P mutant (line O2) αS under the control of a mouse prion protein promoter have been described previously (Lee et al. 2002 PNAS 99, 8968-8973). The mice from line G2-3 develop progressive neurological dysfunction in 12-16 months of age, which rapidly progress to end stage paralysis within 14-21 days following initial onset of symptoms (Lee et al. 2002 PNAS 99, 8968-8973). For this study, Tg mice were bred to establish neuronal cultures, acute slice electrophysiology, biochemical analysis, and behavioral analysis. Mouse genotype was confirmed by Northern blot and reverse transcription-PCR analysis as previously described (see Analysis of Transgene Expression; Lee et al. 2002 PNAS 99, 8968-8973). For all experiments, data was collected from animals of both sexes. All experimental protocols involving mice and rats were in strict adherence to the NIH Animal Care and Guidelines and were approved by the Institutional Animal Care and Use Committee at the University of Minnesota.

### Biochemistry: Gel Electrophoresis and Immunoblotting

Hippocampi from Tg mice were suspended and mechanically homogenized in 10 volumes of ice-cold TNE Buffer (50 mM Tris-HCl, 150 mM NaCl, 5 mM EDTA, complete Mini Protease Inhibitor Cocktail, and Phosphatase Inhibitor Cocktails 2 and 3—inhibitors 1:100, Sigma Aldrich, St. Louis, Mo.) in a polystyrene tube. Homogenized tissue was aliquoted and diluted with equal volumes of ice-cold Complete TNE (TNE, 1% sodium dodecyl sulfate, 0.5% Nonidet P-40, 0.5% sodium deoxycholate). Estimation of protein concentration for protein correction and dilution was performed utilizing the bicinchoninic acid (BCA) assay (Thermo Fisher Scientific, Waltham, Mass.).

Concentration-corrected protein samples were diluted in reducing sample buffer (Boston BioProducts, Ashland, Mass.), electrophoresed on 4-20% Criterion TGX gels (Bio-Rad Laboratories, Hercules, Calif.) and transferred onto Amersham 0.45 μm nitrocellulose membranes (GE Healthcare, Chicago, Ill.). Membranes were probed with primary antibodies of total αS (Catalog No. 610787, BD Biosciences, San Jose, Calif.), human αS (HuSyn1; Lee et al. 2002 PNAS 99, 8968-8973), and α-tubulin (Catalog No. ab4074, Abcam, Cambridge, UK) and visualized utilizing enhanced chemiluminescent reagents (Thermo Fisher Scientific, Waltham, Mass.) via ImageQuant LAS 4000 detection system (GE Healthcare, Chicago, Ill.). Densitometry analysis was performed utilizing ImageQuant TL 8.1 software (GE Healthcare, Chicago, Ill.).

### Acute Slice Electrophysiology

Acute coronal hippocampal slices (350 μm thick) were obtained from 3-6 month old non-transgenic (TgNg) and Tg mice from lines 12-2 (WT), H5 (A53T), G2-3 (A53T), and O2 (A30P). Slices were kept in ice-cold artificial cerebrospinal fluid (ACSF) containing (in mM): NaCl 124, KCl 5, NaH₂PO₄ 1.25, MgSO₄ 2, NaHCO₃ 26, CaCl₂ 2 and glucose 10, gassed with 95% O2/5% CO₂ (pH=7.3-7.4). Slices were incubated in ACSF at room temperature for at least 1 hour before use and then they were transferred to an immersion recording chamber, superfused at 2 mL/minute with gassed ACSF and visualized under an Olympus BX50WI microscope (Olympus Optical, Japan). Picrotoxin (50 μM) and CGP54626 (1 μM) were added to the solution to block GABAa and GABAb receptors respectively. Whole-cell electrophysiological recordings were obtained from CA1 pyramidal neurons. Patch electrodes (3-10 MΩ) were filled with internal solution containing (in mM): cesium-gluconate 117, HEPES 20, EGTA 0.4 NaCl 2.8, TEA-Cl 5, ATP-Mg+2 2, GTP-Na+0.3 (pH=7.3). Recordings were obtained with PC-ONE amplifiers (Dagan Instruments, Minneapolis, Minn.). Membrane potential was held at −70 mV. Signals were filtered at 1 kHz and acquired at 10 kHz sampling rate and fed to a Pentium-based PC through a DigiData 1440A interface board. The pCLAMP 10.4 (Axon Instruments, Molecular Devices, San Jose, Calif.) software was used for stimulus generation, data display, acquisition and storage. To record evoked excitatory postsynaptic currents (EPSCs), theta capillaries filled with ACSF were used for bipolar stimulation and placed in the stratum radiatum to stimulate Schaffer collaterals (SC). Input-output curves of EPSCs were made by increasing stimulus intensities from 20 μA to 80 μA. Paired pulses (2 ms duration) were applied in the SC with 25 ms, 50 ms, 75 ms, 100 ms, 200 ms, 300 ms, and 500 ms interpulse intervals and the paired-pulse ratio was calculated (PPR=2nd EPSC/1st EPSC). Synaptic fatigue was assessed with 15 consecutive stimuli with 25 ms interval. AMPA currents were obtained at a holding potential of −70 mV and NMDA currents at +30 mV. To ascertain the AMPA to NMDA receptor current ratio the NMDA component was measured 50 ms after the stimulus, when the AMPA component had decayed. For long-term potentiation (LTP) induction a tetanic stimulation (4 trains at 100 Hz for 1 second; 30 second intervals) was applied in the SC. EPSC amplitude was normalized to 10 min of baseline recording. After LTP induction, neurons were recorded for 45 minutes. The presence of LTP was determined by comparing the last 5 minutes of baseline with the last 5 minutes of recording. For miniature EPSC recordings TTX (1 μM) was also included in the solution. Normality was verified with a Kolmogorov-Smirnov test in analyses of cumulative curves and groups were compared using a one-way ANOVA with Fisher LSD post-hoc analysis. When data did not meet normality, a one-way Kruskal-Wallis test with Dunn's method post-hoc was applied.

### Plasmid Constructs

All eGFP, tau, αS and DsRed constructs were expressed in the pRK5 vector and driven by a cytomegalovirus promoter (Takara Bio USA (formerly known as Clontech Laboratories, Inc.), Mountain View, Calif.). All tau and αS constructs were tagged with eGFP on the N-terminus. The WT tau construct encodes human four-repeat tau lacking the N-terminal sequences (0N4R) and contained exons 1, 4 and 5, 7, and 9-13, intron 13, and exon 14. Using WT tau as a template, QuickChange site-directed mutagenesis (Agilent Technologies, Santa Clara, Calif.) was used to generate two tau constructs termed AP tau and E14 tau. All 14 S/P or T/P amino acid residues (T111, T153, T175, T181, S199, 5202, T205, T212, T217, T231, S235, S396, S404, and S422) were mutated to alanine (AP) or glutamate (E14). Numbering is based on the longest (2N4R) 441-amino acid adult brain isoform of human tau. All tau constructs were characterized in Hoover et al. 2010 Neuron 68:1067-1081. Site directed mutagenesis was used to generate A30P, E46K, and A53T αS from WT αS. All sequences were confirmed with Sanger Sequencing (UMN Genomics, Minneapolis, Minn.).

### High-Density Neuronal Cultures and Neuronal Transfection

A 25 mm glass polylysine-coated coverslip (thickness, 0.08 mm) was glued to the bottom of a 35 mm culture dish with a 22 mm hole using silicone sealant as previously described (Lin et al. 2004 Biochem Biophys Res Commun. 316(2):501-11). Dissociated neuronal cultures from mouse and rat hippocampi at postnatal day one were prepared as previously described (Hoover et al. 2010 Neuron 68:1067-1081). Briefly, hippocampi were dissected and stored in ice-cold Earl's Balance medium supplemented with 1 mM D-glucose. Rat hippocampal neurons from each litter were pooled before plating; whereas mouse hippocampal neurons were separated by pup before plating. Neurons were plated onto prepared 35 mm culture dishes at a density of 1×10⁶ cells per dish. The age of cultured neurons was counted from the day of plating as one day in vitro (DIV). All experiments were performed on neurons from at least 3 independent cultures. Neurons at 6-8 DIV were transfected with appropriate plasmids using the standard calcium phosphate precipitation method as previously described (Liao et al. 2005 PNAS. 102(5): 1725-30). After transfection, neurons were placed in a tissue culture incubator (37° C., 5% CO₂) and allowed to mature and develop until three weeks in vitro, a time at which neurons express high numbers of dendritic spines with mature morphologies. Mouse culture genotype was ascertained by Northern blot and reverse transcription-PCR analysis of ex-vivo tail clippings (as described above).

### Low-Density Neuronal Cultures

To detect the distribution of endogenous synaptic proteins with high resolution, low-density neuronal cultures were prepared as previously described with some modifications (Lin et al. 2009 Neuropsychopharmacology 34(9):2097-111). Dissociated neuronal cultures from Tg mouse hippocampi at postnatal days 1-2 were plated into 12-well culture plates at a density of 50,000-100,000 cells per well. Each well contained a polylysine-coated 12 mm glass coverslip. The 12 mm coverslips with 7 DIV low-density cultured neurons were transferred to high-density neuronal cultures in 60 mm dishes (4 coverslips per dish; to encourage survival).

### In Vitro Electrophysiology

Miniature excitatory postsynaptic currents (mEPSC) were recorded from cultured dissociated rat hippocampal neurons at 21-25 DIV with a glass pipette (resistance of ˜5 MΩ) at holding potentials of −55 mV and filtered at 1 kHz with an output gain, a, of 0.5 (mouse culture) and 1 (rat culture) as previously described (Miller et al. 2014 Euro. J. Neurosci. 39: 1214-1224). Briefly, neurons were bathed in artificial cerebrospinal fluid (ACSF) at room temperature (25° C.) with 100 μM APV (an NMDAR antagonist), 1 μM TTX (a sodium channel blocker), and 100 μM picrotoxin (GABAa receptor antagonist), gassed with 95% O2-5% CO₂. The ACSF contained (in mM): 119 NaCl, 2.5 KCl, 5.0 CaCl₂, 2.5 MgCl₂, 26.2 NaHCO₃, 1 NaH₂PO₄, and 11 glucose. The internal solution in the patch pipette contained (in mM) 100 cesium gluconate, 0.2 EGTA, 0.5 MgCl₂, 2 ATP, 0.3 GTP, and 40 HEPES (pH 7.2 with cesium hydroxide). mEPSC traces were recorded using Axopatch 200B amplifier and pClamp 11 (Molecular Devices, San Jose, Calif.). Recordings ranged from 5-20 minutes and stable traces longer than 2 minutes in duration were analyzed. All mEPSCs>3 pA were manually counted with MiniAnalysis (Synaptosoft Inc., Fort Lee, N.J.). Each mEPSC event was visually inspected and only events with a distinctly fast-rising phase and a slow-decaying phase were accepted. Relative cumulative frequencies were derived from individual events and the averaged parameters from each neuron were treated as single samples in any further statistical analyses.

### In Vitro Neuronal Imaging and Analysis

The 35 mm culture dishes fit tightly in a custom holding chamber on a fixed platform above an inverted Nikon microscope sitting on a BURLEIGH X-Y translation stage. A 60× oil lens was used for all imaging experiments. Original images were 157.3 μm wide (x-axis) and 117.5 μm tall (y-axis). The z-axis was composed of 15 images, taken at 0.5 μm intervals. All digital images were analyzed with MetaMorph Imaging System (Universal Imaging Co., Molecular Devices, San Jose, Calif.). Unless stated otherwise, live image stacks were processed by 2D deconvolution of nearest planes and averaged into a single image. Dendritic protrusions, with an expanded head that was greater than 50% wider than its neck, were defined as spines. The number of spines from a dendrite was manually counted and normalized per 100 μm dendritic length.

### Immunocytochemistry in Fixed Tissues

Cultured neurons were fixed and permeabilized successively with 4% paraformaldehyde, 100% methanol, and 0.2% Triton X-100 (Lin et al. 2009 Neuropsychopharmacology 34(9):2097-111). For all immunocytochemical staining, primary antibodies were diluted at 1:50 or 1:100 in 10% donkey serum in PBS and rhodamine (red)- or FITC (green)-labeled secondary antibodies were diluted at 1:100 or 1:200 respectively. Mouse anti-synaptophysin (Thermo Fisher Scientific, Waltham, Mass.) antibodies (1:100 dilution) were used to detect presynaptic terminals. Commercial antibodies against PSD-95 were used as a postsynaptic marker to stain dendritic spines (rabbit polyclonal, Invitrogen, Carlsbad, Calif.; mouse monoclonal, Millipore, Burlington, Mass.; 1:100 dilution) as previously described (Lin et al. 2009 Neuropsychopharmacology 34(9):2097-111). The rabbit polyclonal antibodies against the N-terminus of GluA1 subunits were generous gifts from Dr. Richard Huganir (Johns Hopkins University Medical School). The fixed neurons were incubated with primary antibodies at 4° C. overnight and subsequently incubated with secondary antibodies for 1-2 hours at room temperature (PSD-95) or a 37° C. incubator (synaptophysin). The fluorescent images of antibody staining and transfected exogenous proteins (eGFP-αS or eGFP alone) were taken with an inverted Nikon microscope (see In vitro Neuronal Imaging and Analysis above). In FIG. 4A-FIG. 4B, the number of synaptophysin clusters and their colocalization with boutons of neurons expressing eGFP or eGFP-labeled αS were automatically counted using ImageJ software (available on the world wide web at imagej.nih.gov/ij/). For DAPI stained neurons, paraformaldehyde-fixed neurons were incubated in 10 mM DAPI dilactate (Thermo Fisher Scientific, Waltham, Mass.) at 23° C. for five minutes before imaging. DAPI stained nuclei were manually counted from wide-field photomicrographs.

### Barnes Maze Learning and Memory Test

Spatial learning and memory was evaluated using the Barnes Maze as previously described with some modifications (protocol available on the world wide web at nature.com/protocolexchange/protocols/349). The Barnes Maze with video tracking system was purchased from San Diego Instruments (San Diego, Calif.). ANY-maze video tracking software (Stoelting Co., Wood Dale, Ill.) was used for behavioral analysis. Briefly, the maze consists of 20 exploration holes with only one hole leading to a recessed escape box during task acquisition, on an elevated platform (FIG. 2). In each trial, an 11-12 month old mouse was first placed under a box in the center of the maze for about 15 seconds and then allowed to freely explore the maze to search for the escape hole (target) for 3 minutes after the removal of the box. An escape from the maze was defined as the movement of the mouse completely through the escape hole into the recessed box. In the acquisition period (learning phase), the mouse underwent four trials per day with inter-trial interval of 25-30 minutes for four consecutive days. The retention of memory (probe test), was performed 24 hours following the fourth day of acquisition by covering all holes and occupancy plots as the exploration pattern for each group of mice was determined. Retention of memory was measured by quantifying the time that the mouse spent in each zone and the distance from the animal to the position of the removed escape hole (the target) during this 90 second probe test.

### Flow Cytometry

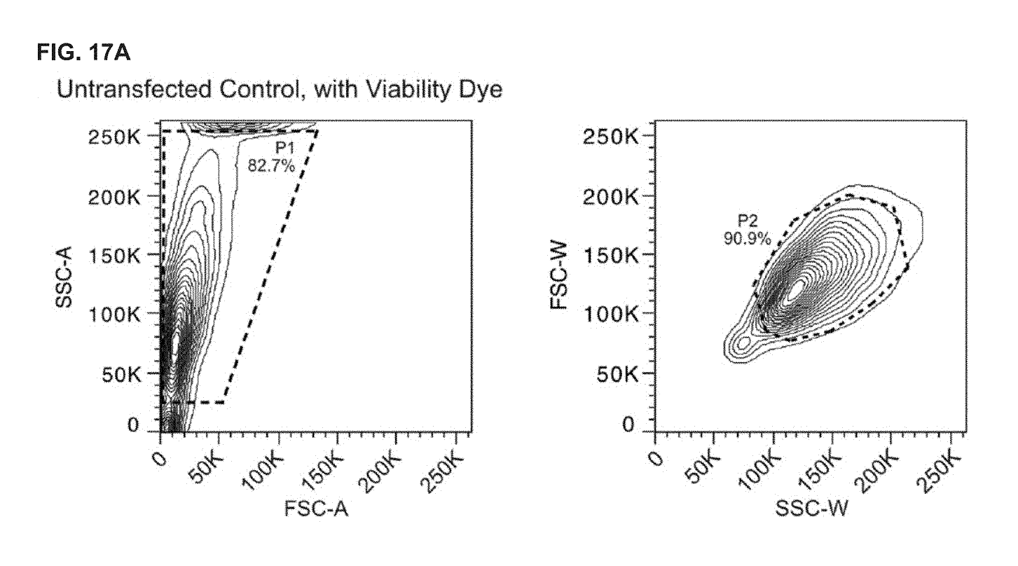

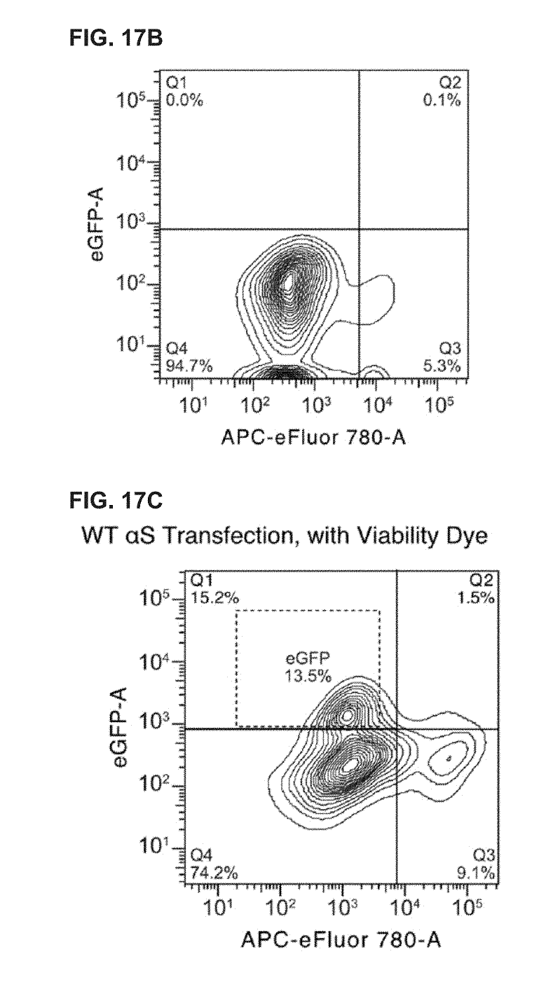

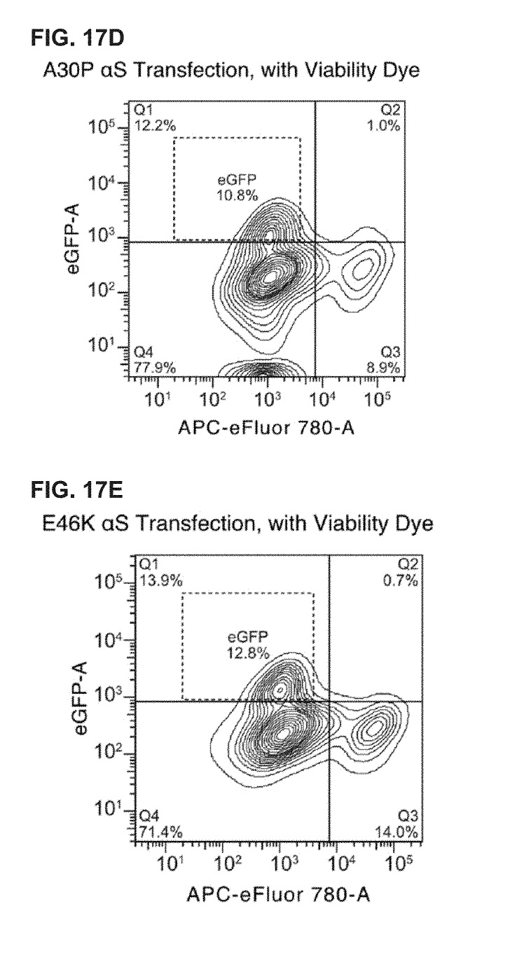

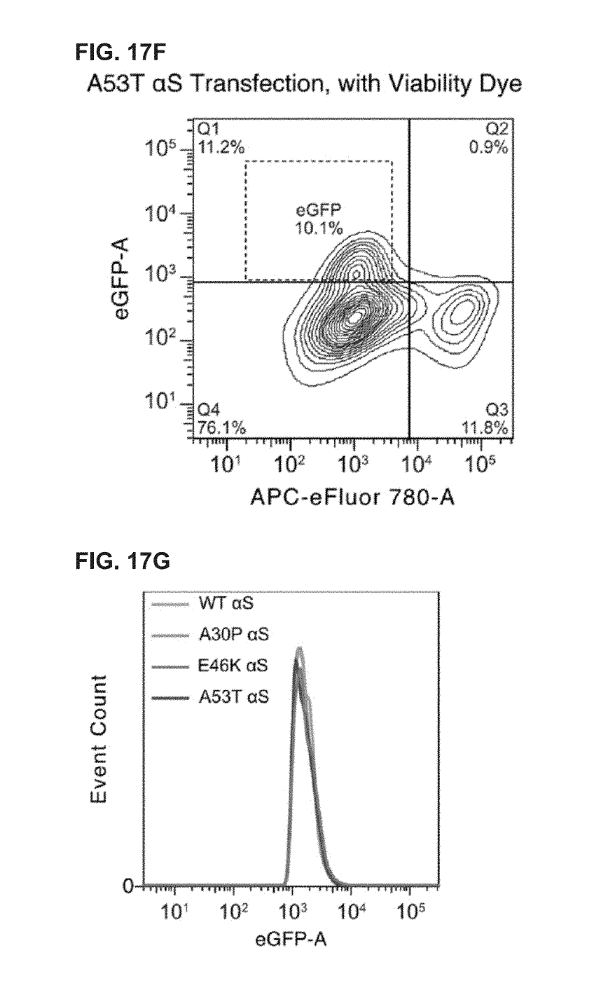

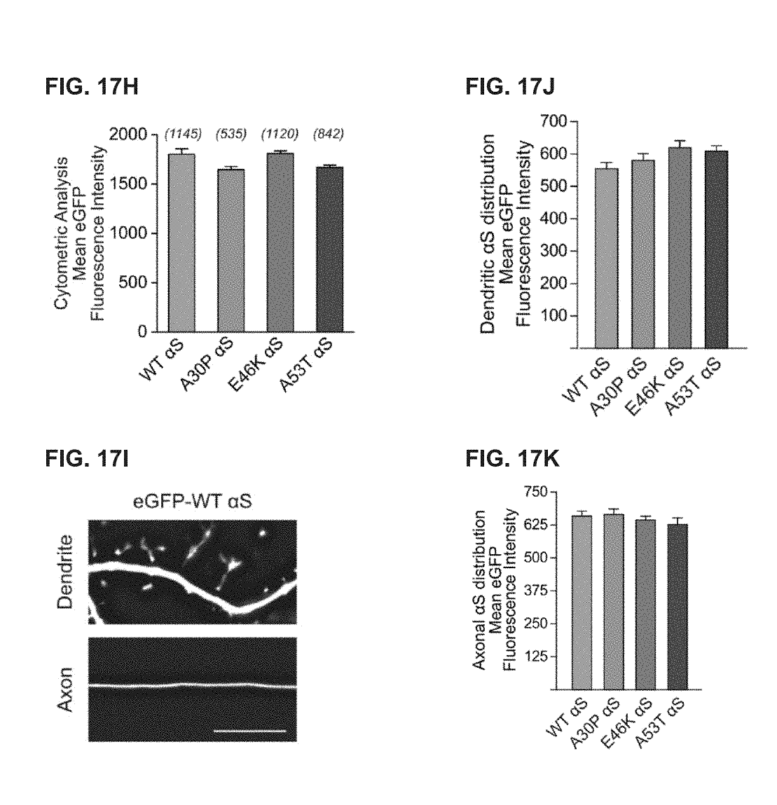

21 DIV rat hippocampal neurons transfected with eGFP-tagged exogenous αS species were suspended, stained for viability, and analyzed via flow cytometry (FIG. 17). Briefly, neurons were washed in 37° C. PBS, then incubated for 6 minutes in 0.05% Trypsin/EDTA (Thermo Fisher Scientific, Waltham, Mass.) at 25° C. with gentle shaking to detach cells. Suspended cells were manually triturated and MEM+10% fetal bovine serum (FBS) 1× GlutaMAX (Thermo Fisher Scientific, Waltham, Mass.) was added to inactivate trypsin. Cells were pelleted by centrifugation (1,000 G, 4° C., 3 minutes), resuspended in phosphate buffered saline (PBS)+2% FBS, then passed through a 70 μm strainer (Thermo Fisher Scientific, Waltham, Mass.) and reserved at 4° C. Cells were pelleted as before, washed once with 1 mL PBS, then resuspended in 50:1 staining buffer (Catalog No. 420201, BioLegend, San Diego, Calif.) and Ghost Dye Red 780 (Tonbo Biosciences, San Diego, Calif.). Cells were incubated on ice for 30 minutes, pelleted, and washed twice with staining buffer. Finally, cells were resuspended in PBS+0.1% bovine serum albumin, passed through a 35 m strainer and analyzed on a BD LSR II Flow Cytometer (BD Biosciences, San Jose, Calif.). Data were analyzed in FlowJo (version 7.6.5, FlowJo LLC, Ashland, Oreg.), with gating parameters represented in FIG. 17.

### Pharmacology and Common Reagents

CHIR-99021 and FK506 were purchased from Sigma Aldrich (St. Louis, Mo.). Both drugs were prepared as stock solutions (CHIR-99021: 5 mM and FK506 1 mM) in fresh DMSO and stored at −20° C. in aliquots. Either drug or DMSO vehicle were applied to cultured cells on DIV 16 with appropriate dilutions, five days prior to imaging or electrophysiology experiments.

### Experimental Design and Statistical Analysis

All statistics were performed in Prism 6 (Graphpad Software, San Diego, Calif.) or Origin (OriginLab, Northampton, Mass.) software. Except where discussed above, one- and two-way ANOVA were used for univariate and two-variable analysis respectively. If ANOVA revealed significant variance between all groups, post-hoc analysis was performed using Bonferroni analysis adjusted for multiple groups. Univariate cumulative frequency distributions were compared using the unmodified Kolmogorov-Smirnov goodness of fit test. For all, statistical significance was set for α=0.05. Data representations are described in respective figure legends.

### Results

### Human A53T αS Induces Mutation-Specific Synaptic Deficits and Spatial Memory Dysfunction

Although most PD cases are sporadic, familial PD can be caused by the duplication or triplication of the WT αS gene (SNCA) as well as point mutations, including the A53T or A30P mutation. Tg mouse lines expressing WT and mutant αS at various levels were used to test effects of these genetic mutations on synaptic responses. Western blots were used to determine the expression levels of both mouse and human αS in four mouse lines: 12-2 mice expressing WT human αS, H5 mice expressing A53T human αS at a lower level, G2-3 mice expressing A53T human αS at a higher level, and O2 mice expressing A30P human αS (FIG. 13A-FIG. 13C, Lee et al. 2002 PNAS 99, 8968-8973). Transgenic negative (TgNg) littermates of 12-2 mice were used as a control.

Next, whole-cell patch-clamp recordings of CA1 pyramidal neurons were performed in acute hippocampal slices from 3- to 6-month-old mice from each line (FIG. 13D-FIG. 13J). Analysis of evoked synaptic responses showed that the input-output curve of all Tg neurons were comparable to TgNg neurons (FIG. 13D) and there was no significant difference in paired-pulse facilitations (FIG. 13E) and synaptic fatigue (FIG. 13F), suggesting that there was no overt degeneration. However, there was a significant reduction in AMPA to NMDA receptor current ratios in hippocampi of H5 and G2-3 mice (FIG. 13G). Overexpression of either WT or A30P αS had no significant effect on the AMPA to NMDA receptor current ratios (FIG. 13G). To further characterize the pre- and postsynaptic changes, mEPSCs were recorded in acute slices (FIG. 13I-FIG. 13J). Consistent with the potential loss of AMPA receptor response, expression of A53T αS, but not WT or A30P αS, significantly decreased the amplitude of AMPA receptor mediated mEPSCs recorded in hippocampal slices (FIG. 13I). These results indicate A53T αS expression is unique in its ability to produce postsynaptic deficits. By contrast, the expression of all three forms of αS (WT, A53T and A30P) significantly decreased the frequency of mEPSCs (FIG. 13J), suggesting a decrease in the release probability of presynaptic vesicles. The specific postsynaptic deficits caused by A53T αS expression and non-specific presynaptic deficits associated with all αS variants imply that pre- and postsynaptic deficits are mediated through two separate intracellular mechanisms.

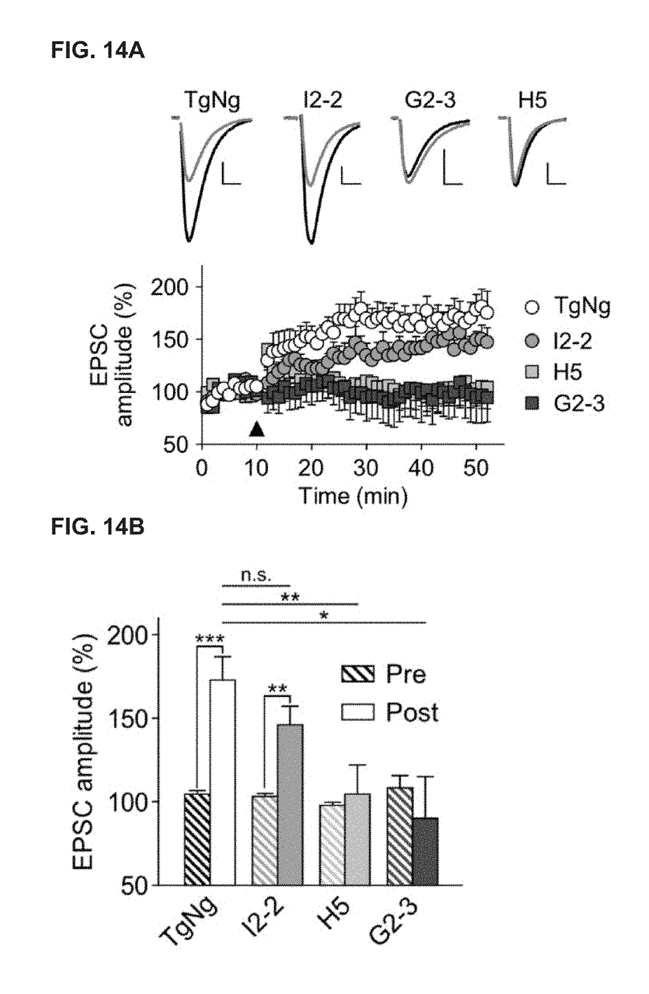

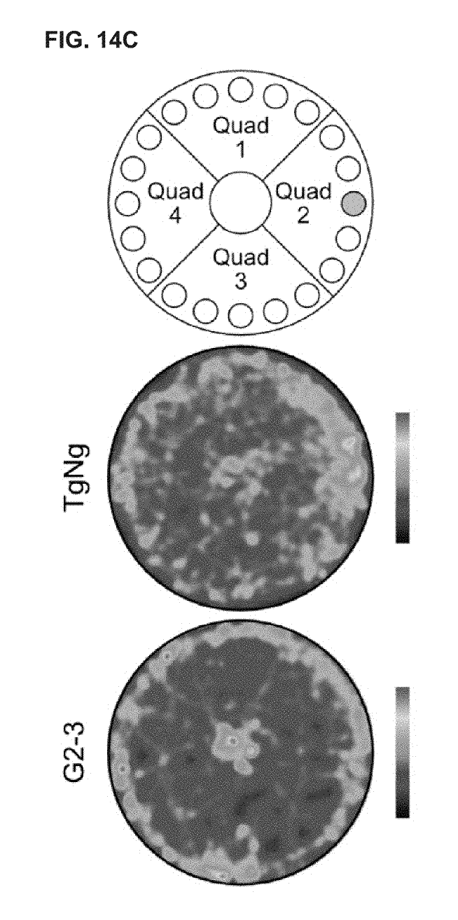

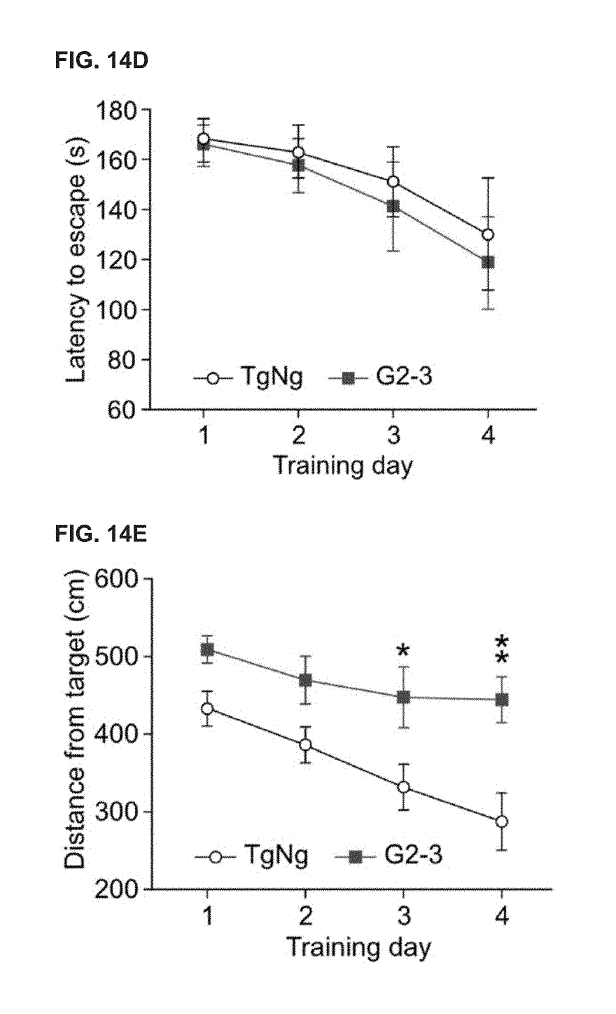

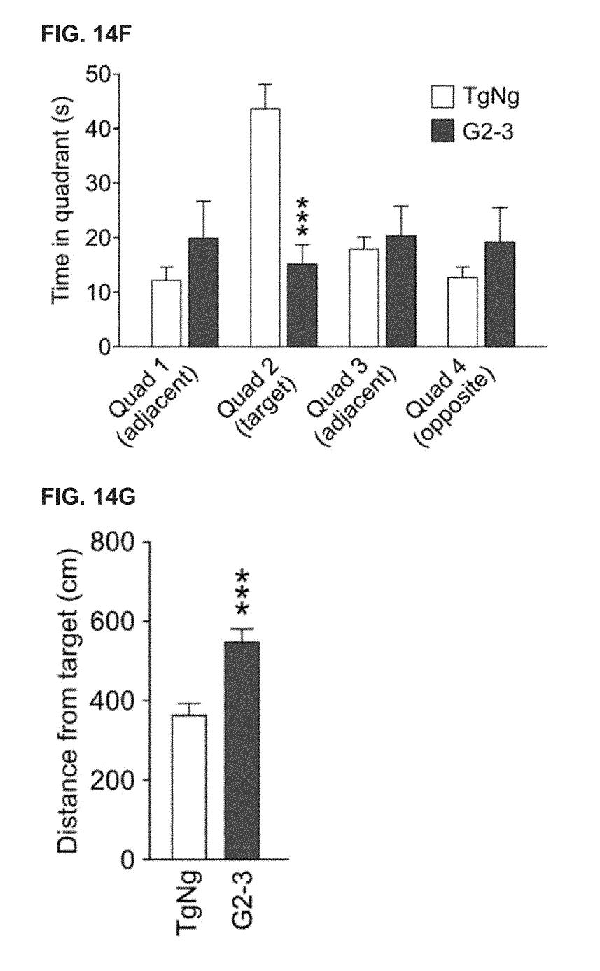

Synaptic plasticity such as long-term potentiation (LTP) is known to increase the synaptic recruitment of AMPA receptors to dendritic spines. Therefore, the results in FIG. 13 may be associated with deficits in LTP and memory. LTP was induced in acute hippocampal slices from 3- to -6-month-old mice expressing WT αS or A53T αS at two expression levels (FIG. 14). Compared to TgNg littermates, lower or higher levels of A53T αS expression suppressed LTP; whereas the expression of WT αS had no significant effect (FIG. 14A-B). Consistent with prior studies which utilized another Tg A53T αS mouse line (M83; Paumier et al. 2013 PlosOne 8(8):e70274), expression of A53T αS was found to impair spatial memory at 11 to 12 months of age (FIG. 14C-FIG. 14G).

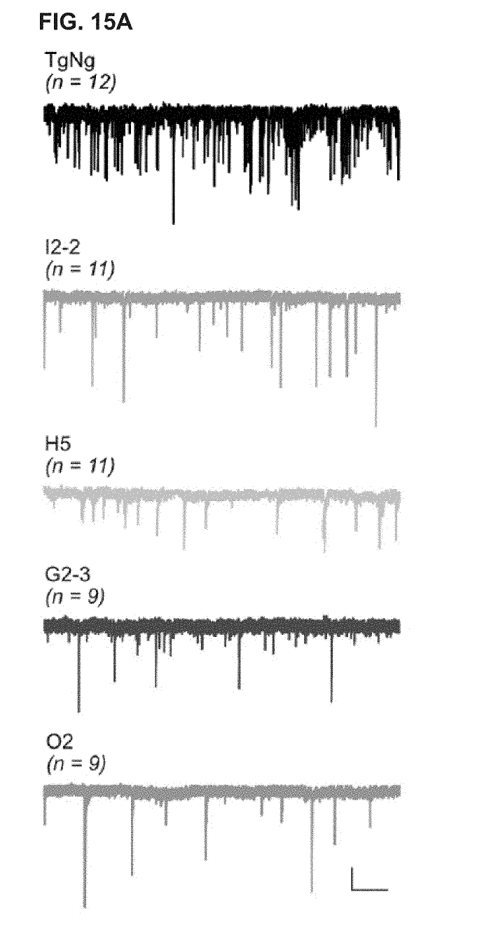

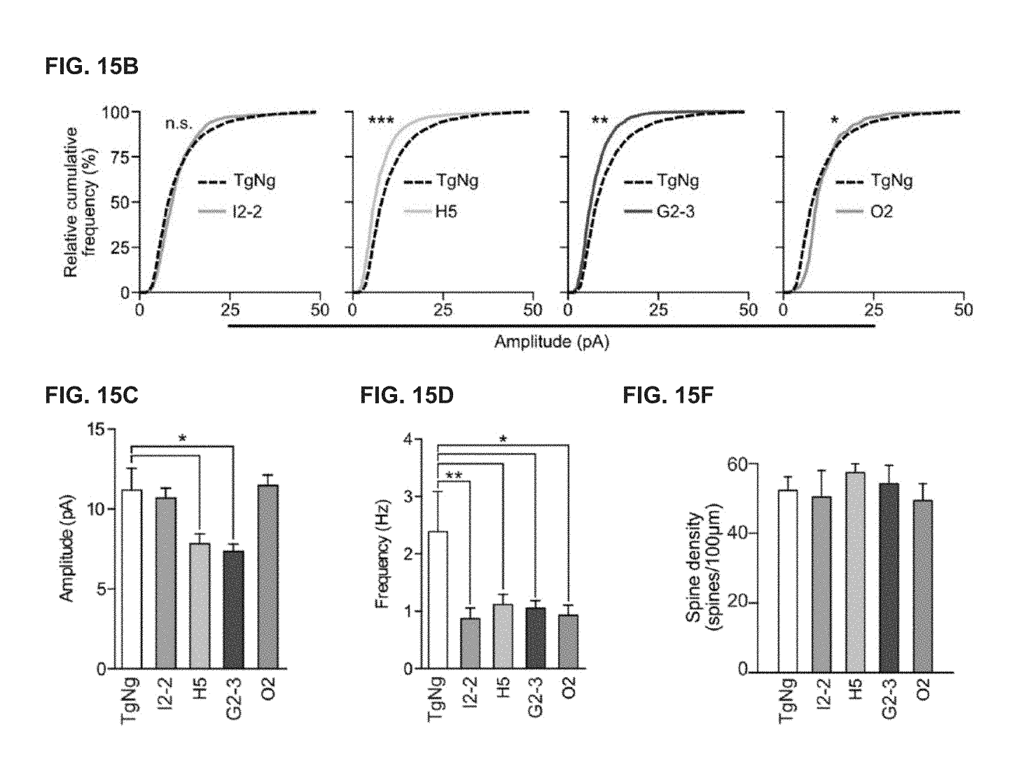

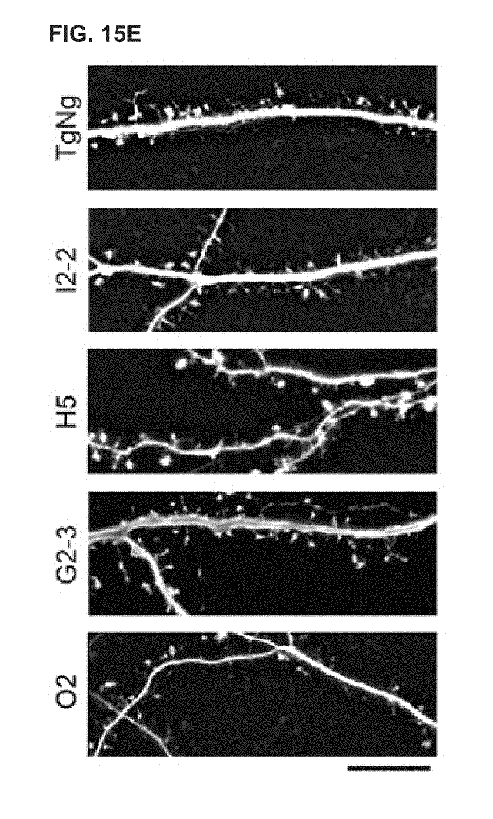

The effects observed in acute hippocampal slices could result from differences in neural circuit development rather than neuron-autonomous differences in postsynaptic responses. Thus, glutamatergic mEPSCs were recorded in cultured hippocampal neurons from TgNg, 12-2, H5, G2-3, and O2 mice (FIG. 15A). The amplitude of mEPSCs was significantly decreased in neurons from the H5 and G2-3 neurons but was unchanged in 12-2 and O2 neurons (FIG. 15B-FIG. 15C). By contrast, the frequency of mEPSCs was significantly decreased in 12-2, H5, G2-3 and O2 neurons (FIG. 15D), confirming that presynaptic deficits are not mutation specific and are induced by hyperexpression of any of the synuclein species (Nemani et al. 2010 Neuron 65: 66-79). Furthermore, the reduced mEPSC frequency and amplitude is not due to loss of postsynaptic structures as there was no alteration in dendritic spine density (FIG. 15E, FIG. 15F). Again, the human αS expression level is similar between 12-2 and H5 mouse lines and between G2-3 and O2 mouse lines (FIG. 13A). Differences in the expression level cannot, therefore, explain the postsynaptic deficits induced by A53T αS expression.

### Human A53T αS Induces Postsynaptic Deficits in a Cell Autonomous Manner

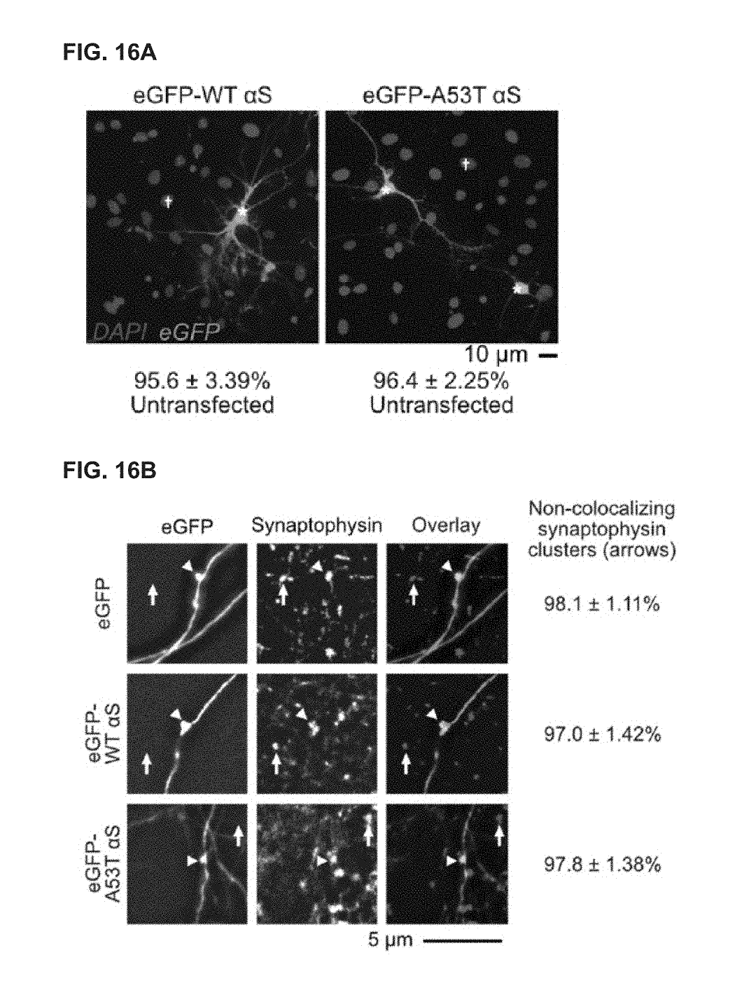

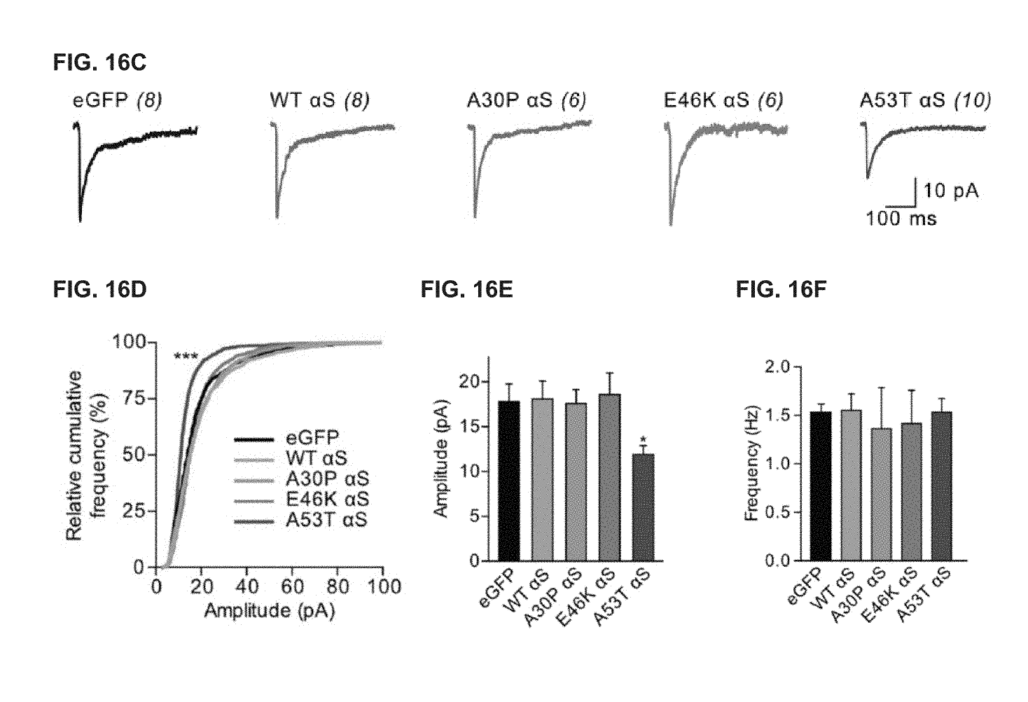

In neuronal cultures established from Tg mice (FIG. 15), αS is present in presynaptic structures, which could lead to secondary postsynaptic deficits. To rule out a presynaptic influence on postsynaptic transmission, calcium phosphate neuronal transfection was used to express exogenous plasmid αS DNA in a small proportion of cells (<5%; FIG. 16A) in rat primary neuronal hippocampal cultures. In this model, neurons expressing the transfected proteins receive presynaptic inputs almost exclusively from terminals that express endogenous proteins alone, that is, no transfected eGFP or eGFP-αS (FIG. 16B). Thus, when patching eGFP-expressing cells any observed postsynaptic changes are cell-autonomous and not associated with the expression of mutant protein in presynaptic neurons. Neurons transfected with eGFP control and eGFP-tagged human WT, A30P, E46K and A53T αS were patched and glutamatergic mEPSCs were recorded (FIG. 16C). Only those neurons expressing A53T αS showed a significant reduction in the amplitude of mEPSCs and expression of the other variants of αS had no significant postsynaptic effect (FIG. 16D, FIG. 16E). By contrast, consistent with the lack of transfected αS expression in presynaptic terminals, postsynaptic expression of the transfected αS variants had no significant effect on mEPSC frequency compared to control (FIG. 16F). These results together indicate that the expression of A53T αS leads to mutation-specific, cell-autonomous postsynaptic dysfunction (FIG. 16). As further control studies, flow cytometry and fluorescence microscopy were employed to compare the expression (FIG. 17A-FIG. 17H) and cellular distribution (FIG. 17I-K) of αS variants in transfected rat neurons, respectively. There was no significant difference between neurons expressing WT, A30P, E46K and A53T αS in the above analyses (FIG. 17), excluding the potential complication that the A53T αS-induced cell-autonomous postsynaptic deficits are due to non-specific changes in expression level.

### A53T αS Induces Phosphorylation-Dependent Tau Mislocalization to Dendritic Spines and Associated Postsynaptic Deficits:

The above results show that only the A53T mutation caused postsynaptic deficits even though both A30P and E46K mutations are linked to autosomal dominant PD. Unlike other kindred with familial PD, one unique pathological feature of PD brains from Contursi kindred, who carry the A53T mutation, is the frequent concurrence of both αS and tau pathology (Duda et al. 2002 Acta Neuropathol. 104(1):7-11). Tau missorting to dendritic spines is associated with memory loss and postsynaptic AMPA receptor signaling in FTDP-17 and Alzheimer's disease. Thus, it was tested whether there was a mechanistic relationship between A53T αS, tau, and postsynaptic deficits. Hippocampal neurons from H5 and G2-3 mice, which express A53T αS, were cultured, and their TgNg littermates were used as the control. These neurons were co-transfected with DsRed and three eGFP-tagged tau constructs (FIG. 18A-FIG. 18C): WT human tau, AP tau (where the 14 proline-directed serine and threonine residues were converted to unphosphorylatable alanine residues), and E14 tau (where the 14 residues were converted to phosphomimetic glutamate) (Hoover et al. 2010 Neuron 68: 1067-1081). The proportion of dendritic spines containing eGFP-tau proteins versus total number of spines, labeled by DsRed, was quantified (Hoover et al. 2010 Neuron 68: 1067-1081; Miller et al. 2014 Euro. J. Neurosci. 39: 1214-1224). Results show that the fraction of dendritic spines containing eGFP-WT tau is significantly higher in the neurons expressing both levels of A53T αS (G2-3 and H5 mice) compared to that in neurons from the TgNg littermates (FIG. 18A, FIG. 18B). By contrast, AP tau does not mislocalize to dendritic spines even when A53T αS is expressed (the 5^{th} bar in FIG. 18B), indicating that tau phosphorylation is necessary for A53T αS-induced mislocalization to dendritic spines. As a positive control, expression of E14 tau causes maximal mislocalization of tau into dendritic spines in both TgNg and G2-3 neurons (bottom two rows in FIG. 6A; right-most two bars in FIG. 18B). The mislocalization of tau is not due to alterations in the neuronal health as the spine density, a sensitive indicator of neurotoxicity, is comparable between all groups (FIG. 18C).

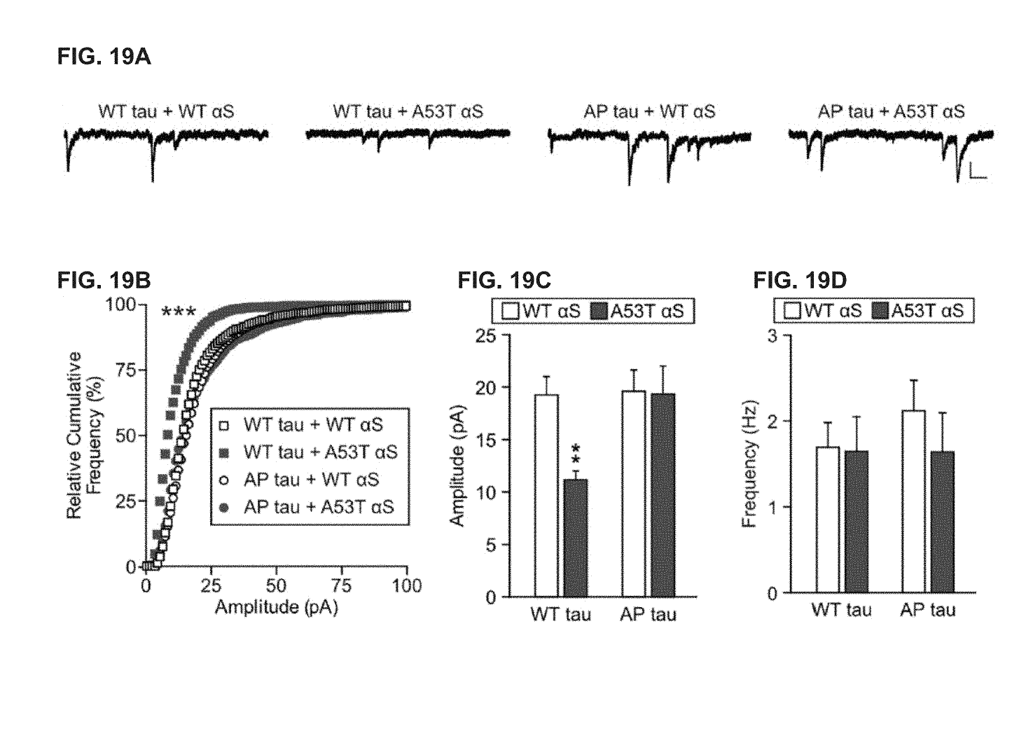

Tau missorting is known to cause functional deficits in dendritic spines, which also depend upon tau phosphorylation. Therefore, it was also tested whether A53T αS-induced synaptic dysfunction is mediated by tau phosphorylation (FIG. 19). As before, calcium phosphate transfection was used to co-transfect cultured rat hippocampal neurons with an αS construct (WT or A53T) and a tau construct (eGFP-tagged WT or AP tau). eGFP-expressing neurons were patched at 20-23 DIV in whole-cell voltage-clamp configuration to record AMPA receptor-mediated mEPSCs. The amplitudes of mEPSCs were significantly lower in neurons co-transfected with WT tau+A53T αS than those in neurons co-transfected with WT tau+WT αS (FIG. 19B, FIG. 19C). However, co-expression of AP tau+A53T αS rescues the mEPSC amplitudes to control levels (FIG. 19B, FIG. 19C), suggesting that tau phosphorylation is required for the A53T αS-induced deficits given that human AP tau may establish a dominant-negative block of endogenous tau. Again, no significant differences in mEPSC frequency were found between the groups (FIG. 19D), confirming that the low transfection rates limit the effect of exogenous protein expression on presynaptic terminals innervating the patched neurons. These results provide a mechanistic link between tau phosphorylation, missorting and A53T αS-induced postsynaptic deficits.

### A53T αS-Induced Tau Missorting and Synaptic Dysfunction Require the Activation of GSK3β

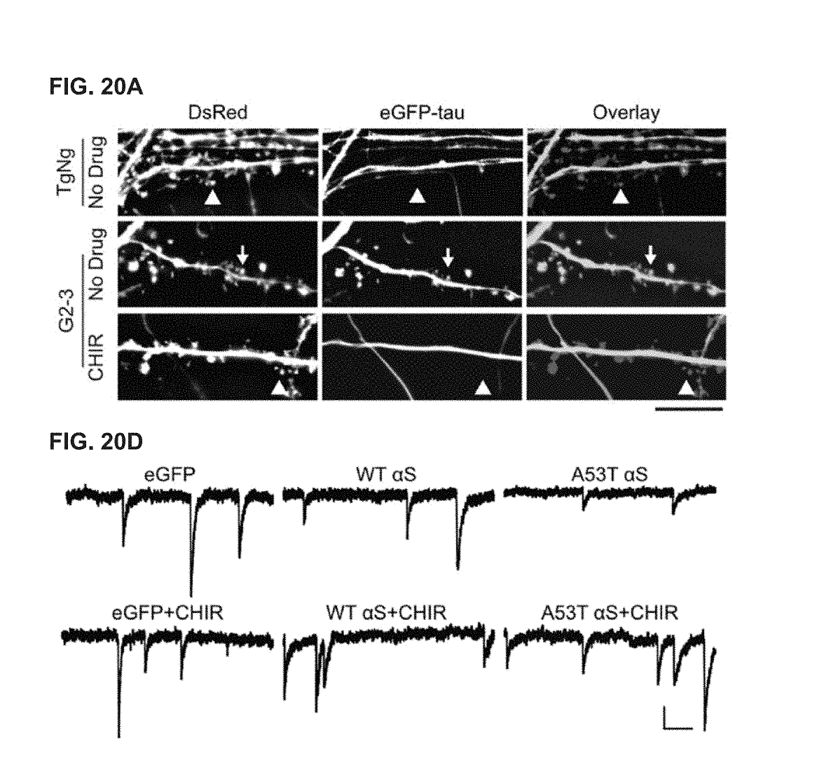

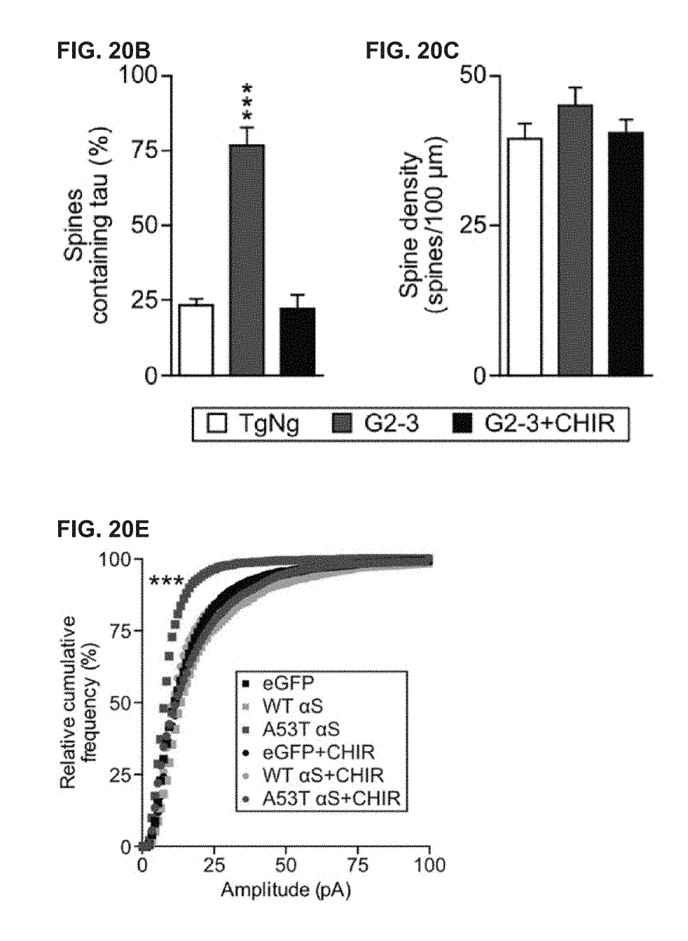

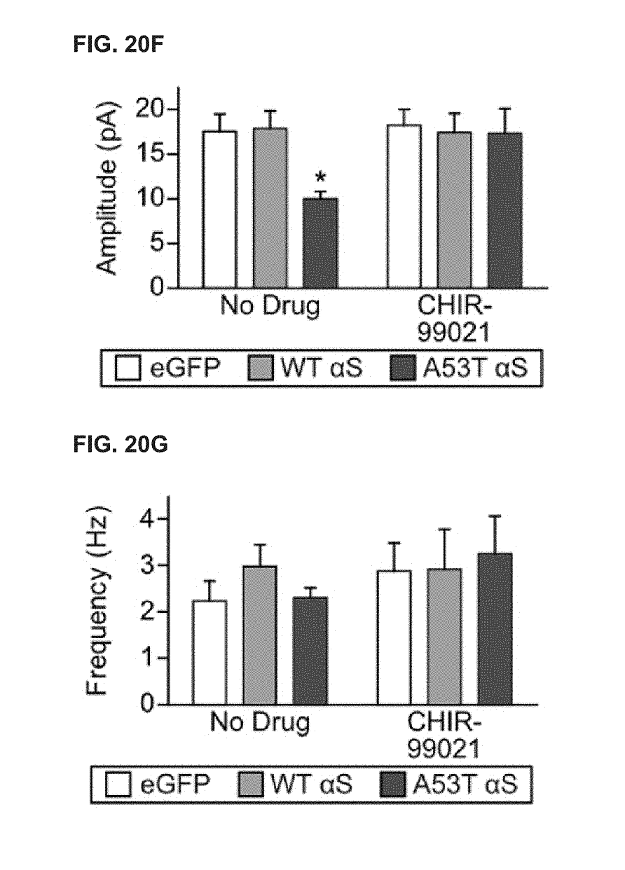

To further clarify the postsynaptic roles of αS, whether pharmacological blockade of αS-initiated tau mislocalization can rescue deficits in AMPA receptor signaling was tested (FIG. 20-FIG. 22). Many kinases have been reported to phosphorylate tau. Among them glycogen synthase kinase 3β (GSK3β) is the most-studied tau kinase in PD pathogenesis, and previous reports indicate that αS can cause GSK3β-mediated tau hyperphosphorylation. Thus, first it was determined whether GSK3β activity is necessary for tau mislocalization in neurons expressing A53T αS (FIG. 20A-FIG. 20C). As above, cultured neurons from G2-3 and TgNg mice were co-transfected with eGFP-WT tau and DsRed. The neurons were treated with 3 μM CHIR-99021 (CHIR), a GSK3β inhibitor, or vehicle at 16 DIV and then imaged the neurons at 21 DIV (FIG. 20A). The increase in dendritic spines containing mislocalized eGFP-tau in G2-3 neurons is blocked by the presence of CHIR (FIG. 20A, FIG. 20B). Therefore, GSK3β activity is necessary for A53T αS-dependent mislocalization of tau to dendritic spines. Furthermore, inhibition of GSK3β can completely reverse the postsynaptic deficits caused by A53T αS transfection (FIG. 20D-FIG. 20F). Collectively, these results indicate that the functional deficits in AMPA receptor-mediated synaptic responses caused by A53T αS require the activity of GSK3β, a major tau kinase.

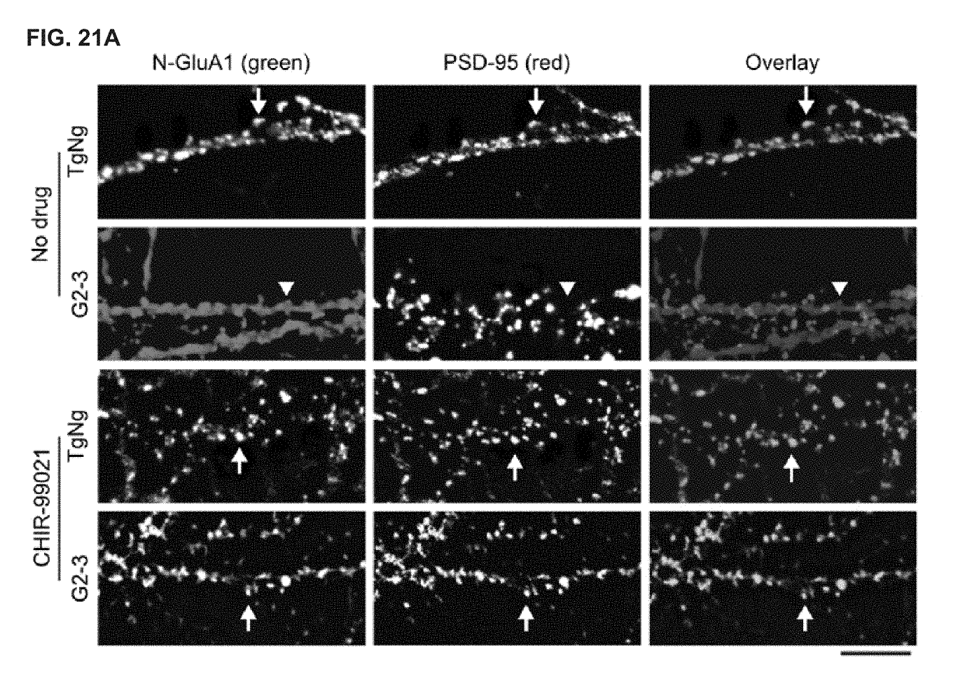

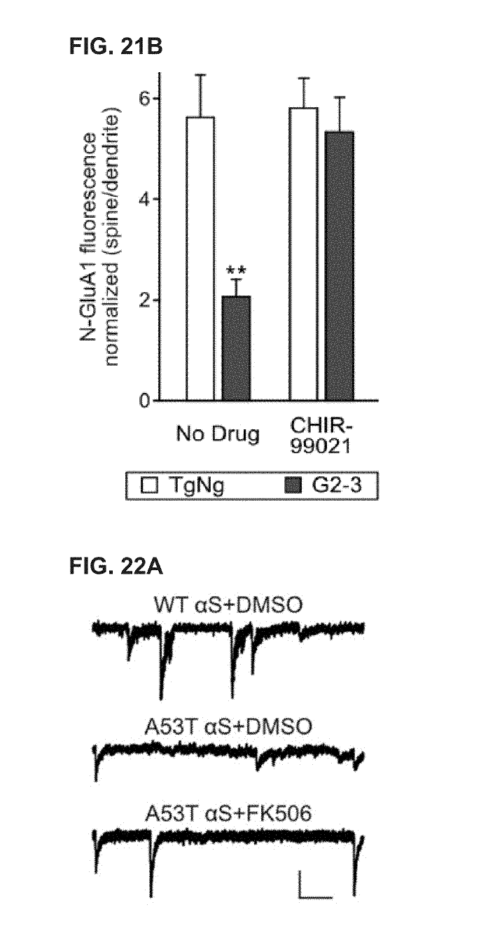

Mislocalization of phospho-tau to dendritic spines leads to reduced mEPSC amplitude by reducing the surface levels of AMPARs. To determine if this reduction in surface levels of AMPARs also occurs with αS-dependent postsynaptic deficits, low density cultures of hippocampal neurons from G2-3 and TgNg mouse lines were treated with CHIR or vehicle and the live neurons were stained with a FITC-conjugated antibody against the N-terminus of GluA1 subunits (N-GluA1; Liao et al. 1999 Nature Neuroscience 2(1):37-43). Next, the neurons were fixed and permeabilized and stained with an antibody against PSD-95 to reveal the location of dendritic spines (see representative images in FIG. 21). Surface GluA-1 signal is normally clustered with PSD-95 at the synapses in mature neurons (>3 weeks in vitro); however, in G2-3 neurons, this colocalization is significantly diminished, leaving only non-specific extrasynaptic staining in the dendritic shaft. When GSK3β activity was blocked with CHIR, strong colocalization of PSD-95 and N-GluA1 clusters is restored (FIG. 21). Together, these results suggest that A53T αS expression causes a GSK3β-dependent decrease in AMPA receptor signaling via postsynaptic internalization of GluA1 subunits or inhibition of synaptic recruitment of these subunits.

### A53T αS-Induced Synaptic Dysfunction Also Requires the Activation of Calcineurin

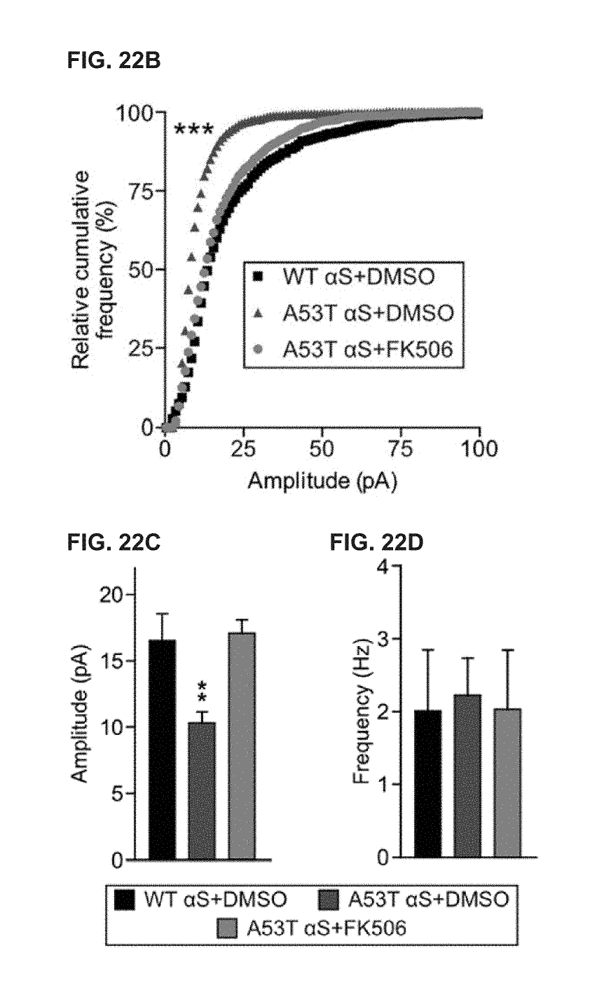

Given that A53T αS causes a loss of surface GluA1, calcineurin-mediated AMPAR internalization was hypothesized to play a role in synaptic deficits caused by A53T αS. Calcineurin is a Ca²⁺-dependent protein phosphatase that mediates AMPAR internalization under multiple conditions including LTD, morphine treatment, neuronal toxicity, and exposure to Aβ. Calcineurin involvement was examined by recording mEPSCs from neurons expressing A53T αS in the presence of the calcineurin inhibitor FK506 (tacrolimus) or the vehicle (FIG. 22). The mEPSC amplitudes in neurons expressing A53T αS treated with vehicle were significantly smaller than those in neurons expressing A53T αS treated with FK506, indicating that calcineurin activation is required for A53T αS-induced synaptic dysfunction (FIG. 22A-FIG. 22C). Again, mEPSC frequency in this low efficiency transfection was unaffected (FIG. 22D), further supporting that the A53T αS-induced calcineurin-mediated changes are mostly postsynaptic and cell autonomous.

### Discussion

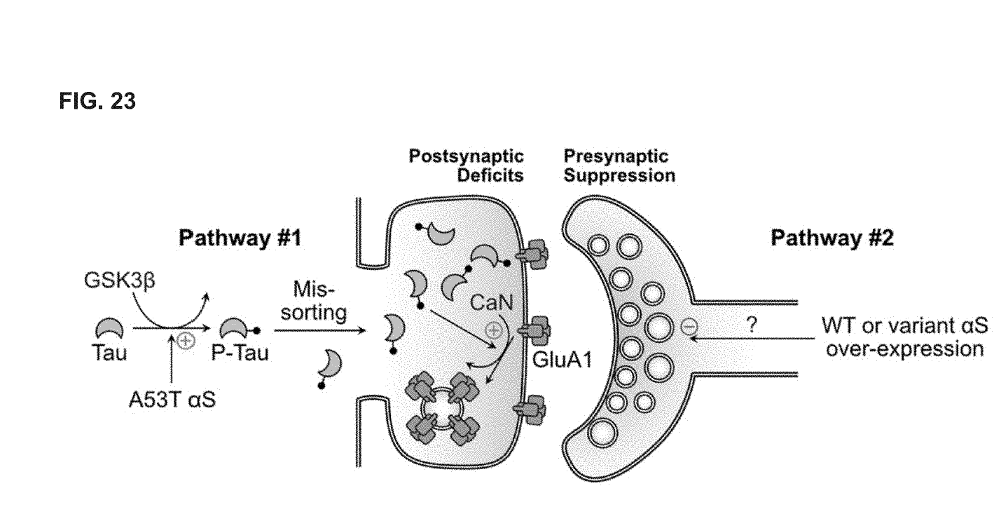

While hyperexpression of WT, A30P, and A53T human αS results in presynaptic deficits, the results of this Example demonstrate that A53T αS causes unique additional deficits in postsynaptic neuronal function (FIG. 13, FIG. 15, and FIG. 16). The presence or absence of postsynaptic deficits may contribute to the clinical and pathological heterogeneity of PD. Unlike the presynaptic deficits, the postsynaptic deficits are not due to a simple increase in αS expression level. Rather, postsynaptic deficits require expression of αS with the specific A53T mutation (FIG. 13, FIG. 15, and FIG. 16). These postsynaptic deficits are likely mediated by a mechanism distinct from that underlying presynaptic deficits. Two separate signaling cascades may be activated by changes in αS. First, A53T αS may cause postsynaptic deficits by inducing tau missorting to dendritic spines (FIG. 23, Pathway #1). Second, an abnormal increase in the expression level of human WT or mutant αS may induce presynaptic deficits by suppressing the release probability of neurotransmitter vesicles (FIG. 23, Pathway #2).

This Example also characterizes a postsynaptic signaling cascade that directly links the A53T αS mutation to tau-dependent pathophysiology (FIG. 23, Pathway #1). These results support the involvement of GSK3b-dependent tau phosphorylation and calcineurin-mediated suppression of AMPA receptor currents in this cascade (FIG. 20 and FIG. 22). It was previously unknown that changes in αS induce tau missorting to dendritic spines and subsequent loss of postsynaptic AMPA receptors. There is strong evidence of frequent concurrence of tau and αS pathologies in the Contursi kindred (Duda et al. 2002 Acta Neuropathol. 104(1):7-11). Additionally, a recent clinical study found that frontotemporal dementia is the presenting phenotype in some A53T carriers with atrophy of prefrontal cortex and elevated tau concentration in cerebrospinal fluid (Bougea et al. 2017 Parkinsonism Related Disorders 35: 82-87). Thus, it is possible that the A53T mutation has a unique pathogenic association with tau leading to frontotemporal dementia and parkinsonism. It is also possible that the postsynaptic link between αS and tau revealed here plays a role in the pathogenesis of some cases of sporadic PD.

Consistent with a previous report by Paumier et al. (2013 PlosOne 8(8):e70274), w aged G2-3 mice were found to exhibit deficits in synaptic plasticity and spatial memory tests. However, the studies reported herein extend the previous analysis by describing a novel molecular basis for synaptic deficits caused by A53T αS (that is, tau-missorting and postsynaptic deficits). Tau missorting to dendritic spines has been shown to be associated with cognitive deficits in models of Alzheimer's disease, FTDP-17, and stress. These results suggest that A53T mutation-induced tau missorting may contribute to dementia observed in the Contursi kindred. However, A53T-linked familial PD is not uniquely associated with dementia; dementia is seen in humans with SNCA multiplications and E46K mutations as well. It is possible that αS abnormalities can cause dementia via multiple mechanisms, which would be consistent with the well-documented clinical heterogeneity of PD. That is, although this study illuminates one possible pathway connecting tau pathology to familial A53T αS PD, it may also be relevant to other synucleinopathies with concurrent tauopathic dementia.

### Example 4

As shown in FIG. 11, AP peptide can block tau missorting to dendritic spines in cultured hippocampal neurons treated with Aβ olgomers. To test the in vivo efficacy of AP peptide for treating Alzheimer's disease, AP peptide will be infused by an osmotic pump into the lateral ventricles of transgenic mice expressing APPsw. The mouse model is described in Hsiao et al. 1996 Science 274:99-102. The perfusion method is described in Zhao et al. 2016 Nat. Med. 22(11): 1268-1276. Tau missorting to dendritic spines will be determined by biochemistry; memory deficits will be evaluated by the Morris water maze behavioral test; and synaptic dysfunction will be assessed by electrophysiology.

Expected outcomes: The AP peptide is expected to block tau missorting to dendritic spines, rescue synaptic deficits, and ameliorate memory deficits, providing direct in vivo evidence that validates the usage of the peptide to treat Alzheimer's disease.

### Example 5

To test the in vivo efficacy of AP peptide for treating FTDP-17, AP peptide will be infused by an osmotic pump into the lateral ventricles of transgenic mice expressing P301L tau. The mouse model is described in Hoover et al. 2010 Neuron 68(6):1067-81. The perfusion method is described in Zhao et al. 2016 Nat. Med. 22(11): 1268-1276. Tau missorting to dendritic spines will be determined by biochemistry; memory deficits will be evaluated by the Morris water maze behavioral test and synaptic dysfunction will be assessed by electrophysiology.

Expected outcomes: The AP peptide is expected to block tau missorting to dendritic spines, rescue synaptic deficits, and ameliorate memory deficits, providing direct in vivo evidence that validates the usage of the peptide to treat FTDP-17.

### Example 6

To test the in vivo efficacy of AP peptide for treating Parkinson's disease, AP peptide will be infused by an osmotic pump into the lateral ventricles of transgenic mice expressing A53T α-synuclein. The mouse model is described in Teravskis et al. 2018 J. Neurosci. 38(45):9754-9767 and Lee et al., 2002 Proc Natl Acad Sci USA 99:8968-8973. The perfusion method is described in Zhao et al. 2016 Nat. Med. 22(11): 1268-1276. Tau missorting to dendritic spines will be determined by biochemistry; memory deficits will be evaluated by the Morris water maze behavioral test and synaptic dysfunction will be assessed by electrophysiology.

Expected outcomes: The AP peptide is expected to block tau missorting to dendritic spines, rescue synaptic deficits, and ameliorate memory deficits, providing direct in vivo evidence that validates the usage of the peptide to treat Parkinson's disease.

### Example 7

To test the in vivo efficacy of AP peptide for treating chronic traumatic encephalopathy (CTE), AP peptide will be infused by an osmotic pump into the lateral ventricles of a traumatic brain injury (TBI) model rat. In the TBI model, mechanical deformations of the rat brain are induced with high accuracy and very fast speed. The AP peptide will be infused to the lateral ventricles during the TBI surgery. One to two weeks after surgery, tau missorting to dendritic spines will be determined by biochemistry; memory deficits will be evaluated by the Morris water maze behavioral test; and synaptic dysfunction will be assessed by electrophysiology.

Expected outcomes: The AP peptide is expected to block tau missorting to dendritic spines, rescue synaptic deficits, and ameliorate memory deficits, providing direct in vivo evidence that validates the usage of the peptide to treat CTE.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A peptide comprising a tau peptide, wherein the tau peptide comprises a sequence of amino acids having at least 80% homology to SPVVSGDTS (SEQ ID NO:4), wherein the tau peptide comprises at least 9 amino acids and up to 45 amino acids.

2. The peptide of claim 1, wherein the tau peptide comprises a sequence of amino acids comprising SPVVSGDTS (SEQ ID NO:4), APVVSGDTA (SEQ ID NO:5), or both.

3. The peptide of claim 1, wherein the tau peptide comprises a sequence of amino acids comprising KSPVVSGDTSP (SEQ ID NO:6) or KAPVVSGDTAP (SEQ ID NO:7), or both.

4. The peptide of claim 1, wherein the tau peptide comprises DHGAEIVYKSPVVSGDTSPRHLSNVSST (SEQ ID NO:8).

5. The peptide of claim 1, wherein the tau peptide comprises a mutation that blocks the phosphorylation of at least one of S396 and S404 in tau.

6. The peptide of claim 5, wherein at least one of S396 and S404 of human tau is replaced with an alanine.

7. The peptide of claim 6, wherein the tau peptide comprises DHGAEIVYKAPVVSGDTAPRHLSNVSST (SEQ ID NO: 9).

8. The peptide of claim 1, wherein the tau peptide further comprises
a protein transduction domain, wherein the protein transduction domain is optionally conjugated to the N terminus of the tau peptide, or
a modification to increase its ability to cross the blood-brain barrier, or both.

9. The peptide of claim 8, wherein the protein transduction domain comprises an HIV Trans-Activator of Transcription (TAT) domain.

10. The peptide of claim 9, wherein the protein transduction domain comprises GRKKRRQRRRPQ (SEQ ID NO:10).

11. The peptide of claim 10, wherein the peptide prevents the mislocalization of tau that leads to tau-mediated synaptic deficits.

12. The peptide of claim 1, wherein the peptide reduces the localization of tau to the dendritic spines of a mechanically injured neuron by at least 10 percent.

13. The peptide of claim 1, wherein the peptide comprises GRKKRRQRRRPQDHGAEIVYKSPVVSGDTSPRHLSNVSST (SEQ ID NO: 1), or GRKKRRQRRRPQDHGAEIVYKAPVVSGDTAPRHLSNVSST (SEQ ID NO:2), or both.

14. A method of making the peptide of claim 1.

15. A composition comprising the peptide of claim 1.

16. A method comprising administering the peptide of claim 1 to a subject.

17. The method of claim 16, the method further comprising administering a kinase inhibitor to the subject.

18. A vector encoding the peptide of claim 1.

19. A method comprising administering a tau peptide to a subject, wherein the tau peptide comprises SPVVSGDTS (SEQ ID NO:4), APVVSGDTA (SEQ ID NO:5), or both, and wherein the tau peptide comprises at least 9 amino acids and up to 45 amino acids.

20. The method of claim 19, wherein the subject is at risk of or exhibiting symptoms of Alzheimer's Disease, Parkinson's disease, chronic traumatic encephalopathy, and/or another tauopathy.


SEQUENCE LISTING (13 sequences)

SEQ ID NO: 1 - protein, 40 aa, artificial
  tau peptide
GRKKRRQRRRPQDHGAEIVYKSPVVSGDTSPRHLSNVSST

SEQ ID NO: 2 - protein, 40 aa, artificial
  tau peptide
GRKKRRQRRRPQDHGAEIVYKAPVVSGDTAPRHLSNVSST

SEQ ID NO: 3 - protein, 40 aa, artificial
  tau peptide
GRKKRRQRRRPQDHGAEIVYKEPVVSGDTEPRHLSNVSST

SEQ ID NO: 4 - protein, 9 aa, artificial
  tau peptide
SPVVSGDTS

SEQ ID NO: 5 - protein, 9 aa, artificial
  tau peptide
APVVSGDTA

SEQ ID NO: 6 - protein, 11 aa, artificial
  tau peptide
KSPVVSGDTSP

SEQ ID NO: 7 - protein, 11 aa, artificial
  tau peptide
KAPVVSGDTAP

SEQ ID NO: 8 - protein, 28 aa, artificial
  tau peptide
DHGAEIVYKSPVVSGDTSPRHLSNVSST

SEQ ID NO: 9 - protein, 28 aa, artificial
  tau peptide
DHGAEIVYKAPVVSGDTAPRHLSNVSST

SEQ ID NO: 10 - protein, 12 aa, artificial
  tau peptide
GRKKRRQRRRPQ

SEQ ID NO: 11 - protein, 7 aa, artificial
  tau peptide
PVVSGDT

SEQ ID NO: 12 - protein, 9 aa, artificial
  tau peptide
APVVSGDTS

SEQ ID NO: 13 - protein, 9 aa, artificial
  tau peptide
SPVVSGDTA